(19) United States
(12) Patent Application Publication
(10) Pub. No.: US 2023/0055381 A1
(43) Pub. Date: Feb. 23, 2023

(54) ADENO-ASSOCIATED VIRUS PACKAGING SYSTEMS

(71) Applicant: Oxford Biomedica Solutions LLC, Bedford, MA (US)

(72) Inventors: Laura VAN LIESHOUT, Bedford, MA (US); Marissa STANVICK, Bedford, MA (US)

(21) Appl. No.: 17/808,637

(22) Filed: Jun. 24, 2022

Related U.S. Application Data
(60) Provisional application No. 63/266,646, filed on Jan. 11, 2022.
(60) Provisional application No. 63/262,218, filed on Oct. 7, 2021.
(60) Provisional application No. 63/202,817, filed on Jun. 25, 2021.

(51) Int. Cl. C12N 15/86; C12N 5/07

(52) U.S. Cl. CPC C12N 15/86; C12N 5/0601; A61K 48/00

(57) ABSTRACT

Provided herein is a dual vector transfection system for the production of recombinant adeno-associated virus (rAAV). The dual vector transfection system generally comprises: (1) a first nucleic acid vector comprising a first nucleotide sequence encoding an AAV Rep protein, a second nucleotide sequence comprising an rAAV genome comprising a transgene, and a third nucleotide sequence encoding an AAV capsid protein; and (2) a second nucleic acid vector comprising a helper virus gene.

### RELATED APPLICATIONS

This application claims priority to U.S. Provisional Patent Application Ser. No. 63/202,817, filed Jun. 25, 2021, 63/262,218, filed Oct. 7, 2021, and 63/266,646, filed Jan. 11, 2022, the entire disclosures of which are hereby incorporated herein by reference.

### SEQUENCE LISTING

This application contains a sequence listing which has been submitted electronically in ASCII format and is hereby incorporated by reference in its entirety (said ASCII copy, created on Sep. 20, 2022, is named “ReplacementSequenceListing17808637.txt” and is 337,026 bytes in size).

### BACKGROUND

Adeno-associated virus (AAV) possesses unique features that make it attractive as a vector for delivering foreign DNA into cells for the purposes of gene therapy. Commercial manufacturing of AAV generally employ either mammalian cell or insect cell systems. Commercial mammalian cell-based AAV production systems typically involve transfection of three plasmids into the cells: a first plasmid containing sequences that encode the AAV Rep and AAV capsid proteins; a second plasmid containing the AAV vector genome; and a third plasmid containing one or more helper virus genes (usually adenovirus or herpesvirus genes). Although effective, such three plasmid AAV manufacturing systems are complex to optimize and contribute to the high cost of goods associated with commercial AAV therapeutics.

Accordingly, there is a need in the art for improved AAV manufacturing systems,

The present disclosure provides a dual vector transfection system for the production of recombinant adeno-associated virus (rAAV). The dual vector transfection system described herein generally comprises: (1) a first nucleic acid vector comprising a first nucleotide sequence encoding an AAV Rep protein, a second nucleotide sequence comprising an rAAV genome comprising a transgene, and a third nucleotide sequence encoding an AAV capsid protein; and (2) a second nucleic acid vector comprising a helper virus gene. In such dual vector transfection systems, the first nucleic acid vector and the second nucleic acid vector together with a host production cell provide all the components required for AAV production. It has been found that the dual vector transfection system disclosed herein results in increased rAAV productivity, as compared to conventional triple vector transfection systems. In addition, the specific organization of components in the dual vector transfection systems described herein was found to result in superior rAAV productivity over a prior art dual vector transfection system.

Accordingly, in one aspect, the present disclosure provides a first nucleic acid vector comprising: a first nucleotide sequence encoding an AAV Rep protein; a second nucleotide sequence comprising a recombinant AAV (rAAV) genome comprising a transgene; and a third nucleotide sequence encoding an AAV capsid protein, wherein the nucleic acid vector does not comprise a helper virus gene.

In certain embodiments, the nucleic acid vector comprises from 5′ to 3′: the first nucleotide sequence encoding an AAV Rep protein; the second nucleotide sequence comprising a recombinant AAV (rAAV) genome comprising a transgene; and the third nucleotide sequence encoding an AAV capsid protein, wherein the nucleic acid vector does not comprise a helper virus gene.

In certain embodiments, the nucleic acid vector comprises from 5′ to 3′: the first nucleotide sequence encoding an AAV Rep protein; the second nucleotide sequence comprising a recombinant AAV (rAAV) genome comprising a transgene; and the third nucleotide sequence encoding an AAV capsid protein, wherein the nucleic acid vector does not comprise a helper virus gene, and wherein the transgene is not selected from the group consisting of phenylalanine hydroxylase (PAH), arylsulfatase A (ARSA), iduronate 2-sulfatase (I2S), and an anti-complement component 5 (C5) antibody.

In certain embodiments, the nucleic acid vector comprises from 5′ to 3′: the first nucleotide sequence encoding an AAV Rep protein; the second nucleotide sequence comprising a recombinant AAV (rAAV) genome comprising a transgene; and the third nucleotide sequence encoding an AAV capsid protein, wherein the nucleic acid vector does not comprise a helper virus gene, and wherein the AAV capsid protein does not comprise an amino acid sequence that is at least 95% identical to the amino acid sequence of amino acids 203-736 of SEQ ID NO: 1, 2, 3, 4, 5, 6, 7, 8, 9, 10, 11, 12, 13, 15, 16, or 17, wherein the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G.

In certain embodiments, the nucleic acid vector comprises from 5′ to 3′: the first nucleotide sequence encoding an AAV Rep protein; the second nucleotide sequence comprising a recombinant AAV (rAAV) genome comprising a transgene; and the third nucleotide sequence encoding an AAV capsid protein, wherein the nucleic acid vector does not comprise a helper virus gene, and wherein (i) the transgene is not selected from the group consisting of phenylalanine hydroxylase (PAH), arylsulfatase A (ARSA), iduronate 2-sulfatase (I2S), and an anti-complement component 5 (C5) antibody, and (ii) the AAV capsid protein does not comprise an amino acid sequence that is at least 95% identical to the amino acid sequence of amino acids 203-736 of SEQ ID NO: 1, 2, 3, 4, 5, 6, 7, 8, 9, 10, 11, 12, 13, 15, 16, or 17, wherein the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G.

In certain embodiments, the nucleic acid vector comprises from 5′ to 3′: the first nucleotide sequence encoding an AAV Rep protein; the second nucleotide sequence comprising a recombinant AAV (rAAV) genome comprising a transgene; and the third nucleotide sequence encoding an AAV capsid protein.

In certain embodiments, the nucleic acid vector is a DNA plasmid or a DNA minimal vector.

In another aspect, the present disclosure provides a recombinant AAV (rAAV) packaging system, comprising: (i) a first nucleic acid vector comprising: a first nucleotide sequence encoding an AAV Rep protein; a second nucleotide sequence comprising a recombinant AAV (rAAV) genome comprising a transgene; and a third nucleotide sequence encoding an AAV capsid protein, and (ii) a second nucleic acid vector comprising a helper virus gene.

In certain embodiments, the first nucleic acid vector comprises from 5′ to 3′: the first nucleotide sequence encoding an AAV Rep protein; the second nucleotide sequence comprising a recombinant AAV (rAAV) genome comprising a transgene; and the third nucleotide sequence encoding an AAV capsid protein. In certain embodiments, the transgene is not selected from the group consisting of phenylalanine hydroxylase (PAH), arylsulfatase A (ARSA), iduronate 2-sulfatase (I2S), and an anti-complement component 5 (C5) antibody. In certain embodiments, the AAV capsid protein does not comprise an amino acid sequence that is at least 95% identical to the amino acid sequence of amino acids 203-736 of SEQ ID NO: 1, 2, 3, 4, 5, 6, 7, 8, 9, 10, 11, 12, 13, 15, 16, or 17, wherein the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G. In certain embodiments, the transgene is not selected from the group consisting of phenylalanine hydroxylase (PAH), arylsulfatase A (ARSA), iduronate 2-sulfatase (I2S), and an anti-complement component 5 (C5) antibody, and the AAV capsid protein does not comprise an amino acid sequence that is at least 95% identical to the amino acid sequence of amino acids 203-736 of SEQ ID NO: 1, 2, 3, 4, 5, 6, 7, 8, 9, 10, 11, 12, 13, 15, 16, or 17, wherein the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G.

In certain embodiments, the first nucleic acid vector is a DNA plasmid or DNA minimal vector. In certain embodiments, the second nucleic acid vector is a DNA plasmid or DNA minimal vector.

In certain embodiments, the transgene encodes a polypeptide. In certain embodiments, the transgene encodes an miRNA, shRNA, siRNA, antisense RNA, gRNA, antagomir, miRNA sponge, RNA aptazyme, RNA aptamer, lncRNA, ribozyme, or mRNA. In certain embodiments, the transgene encodes a protein selected from the group consisting of phenylalanine hydroxylase (PAH), glucose-6-phosphatase (G6Pase), iduronate-2-sulfatase (I2S), arylsulfatase A (ARSA), and frataxin (FXN). In certain embodiments, the transgene encodes glucose-6-phosphatase (G6Pase) or frataxin (FXN).

In certain embodiments, the rAAV genome further comprises a transcriptional regulatory element operably linked to the transgene. In certain embodiments, the transcriptional regulatory element comprises a promoter element and/or an intron element.

In certain embodiments, the rAAV genome further comprises a polyadenylation sequence. In certain embodiments, the polyadenylation sequence is 3′ to the transgene.

In certain embodiments, the rAAV genome comprises a nucleotide sequence that is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 71, 85, 86, 87, or 88.

In certain embodiments, the rAAV genome further comprises a 5′ inverted terminal repeat (5′ ITR) nucleotide sequence 5′ of the transgene, and a 3′ inverted terminal repeat (3′ ITR) nucleotide sequence 3′ of the transgene. In certain embodiments, the 5′ ITR nucleotide sequence is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 39, 41, or 42, and/or the 3′ ITR nucleotide sequence is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 40, 43, or 44.

In certain embodiments, the rAAV genome comprises a nucleotide sequence that is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 75, 78, 80, 82, or 84.

In certain embodiments, the AAV Rep protein is a wild-type Rep protein or a variant thereof. In certain embodiments, the AAV Rep protein is an AAV2 Rep protein or a variant thereof.

In certain embodiments, the first nucleotide sequence further comprises a transcriptional regulatory element operably linked to the AAV Rep protein coding sequence. In certain embodiments, the transcriptional regulatory element comprises a promoter selected from the group consisting of a constitutive promoter, an inducible promoter, or a native promoter. In certain embodiments, the promoter is selected from the group consisting of a P5 promoter, a P19 promoter, a metallothionine (MT) promoter, a mouse mammary tumor virus (MMTV) promoter, a T7 promoter, an ecdysone insect promoter, a tetracycline-repressible promoter, a tetracycline-inducible promoter, an RU486-inducible promoter, and a rapamycin-inducible promoter.

In certain embodiments, the AAV capsid protein is selected from the group consisting of AAV1, AAV2, AAV3, AAV4, AAV5, AAV6, AAV7, AAV8, AAV9, AAVrh10, AAVRh32.33, AAVrh74, AAV-DJ, AAV-LK03, NP59, VOY101, VOY201, VOY701, VOY801, VOY1101, AAVPHP.N, AAVPHP.A, AAVPHP.B, PHP.B2, PHP.B3, G2A3, G2B4, G2B5, and PHP.S. In certain embodiments, the AAV capsid protein is selected from the group consisting of AAV1, AAV2, AAV5, AAV6, AAV8, AAV9, AAVrh10 and AAVrh74. In certain embodiments, the AAV capsid protein is selected from the group consisting of AAV1, AAV2, AAV5, AAV6, AAV8 and AAVrh74.

In certain embodiments, the AAV capsid protein comprises an amino acid sequence that is at least 85% identical to the amino acid sequence of amino acids 203-736 of SEQ ID NO: 1, 2, 3, 4, 5, 6, 7, 8, 9, 10, 11, 12, 13, 15, 16, or 17.

In certain embodiments, the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G.

In certain embodiments, (a) the amino acid in the capsid protein corresponding to amino acid 626 of SEQ ID NO: 16 is G, and the amino acid in the capsid protein corresponding to amino acid 718 of SEQ ID NO: 16 is G; (b) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M; (c) the amino acid in the capsid protein corresponding to amino acid 505 of SEQ ID NO: 16 is R, and the amino acid in the capsid protein corresponding to amino acid 687 of SEQ ID NO: 16 is R; (d) the amino acid in the capsid protein corresponding to amino acid 346 of SEQ ID NO: 16 is A, and the amino acid in the capsid protein corresponding to amino acid 505 of SEQ ID NO: 16 is R; or (e) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C.

In certain embodiments, the AAV capsid protein comprises the amino acid sequence of amino acids 203-736 of SEQ ID NO: 1, 2, 3, 4, 5, 6, 7, 8, 9, 10, 11, 12, 13, 15, 16, or 17.

In certain embodiments, the AAV capsid protein comprises an amino acid sequence that is at least 85% identical to the amino acid sequence of amino acids 138-736 of SEQ ID NO: 1, 2, 3, 4, 5, 6, 7, 8, 9, 10, 11, 12, 13, 15, 16, or 17.

In certain embodiments, the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G.

In certain embodiments, (a) the amino acid in the capsid protein corresponding to amino acid 626 of SEQ ID NO: 16 is G, and the amino acid in the capsid protein corresponding to amino acid 718 of SEQ ID NO: 16 is G; (b) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M; (c) the amino acid in the capsid protein corresponding to amino acid 505 of SEQ ID NO: 16 is R, and the amino acid in the capsid protein corresponding to amino acid 687 of SEQ ID NO: 16 is R; (d) the amino acid in the capsid protein corresponding to amino acid 346 of SEQ ID NO: 16 is A, and the amino acid in the capsid protein corresponding to amino acid 505 of SEQ ID NO: 16 is R; or (e) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C.

In certain embodiments, the AAV capsid protein comprises the amino acid sequence of amino acids 138-736 of SEQ ID NO: 1, 2, 3, 4, 5, 6, 7, 9, 10, 11, 12, 13, 15, 16, or 17.

In certain embodiments, the AAV capsid protein comprises an amino acid sequence that is at least 85% identical to the amino acid sequence of amino acids 1-736 of SEQ ID NO: 1, 2, 3, 4, 5, 6, 7, 8, 9, 10, 11, 12, 13, 15, 16, or 17.

In certain embodiments, the amino acid in the capsid protein corresponding to amino acid 2 of SEQ ID NO: 16 is T; the amino acid in the capsid protein corresponding to amino acid 65 of SEQ ID NO: 16 is I; the amino acid in the capsid protein corresponding to amino acid 68 of SEQ ID NO: 16 is V; the amino acid in the capsid protein corresponding to amino acid 77 of SEQ ID NO: 16 is R; the amino acid in the capsid protein corresponding to amino acid 119 of SEQ ID NO: 16 is L; the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G.

In certain embodiments, (a) the amino acid in the capsid protein corresponding to amino acid 2 of SEQ ID NO: 16 is T, and the amino acid in the capsid protein corresponding to amino acid 312 of SEQ ID NO: 16 is Q; (b) the amino acid in the capsid protein corresponding to amino acid 65 of SEQ ID NO: 16 is I, and the amino acid in the capsid protein corresponding to amino acid 626 of SEQ ID NO: 16 is Y; (c) the amino acid in the capsid protein corresponding to amino acid 77 of SEQ ID NO: 16 is R, and the amino acid in the capsid protein corresponding to amino acid 690 of SEQ ID NO: 16 is K; (d) the amino acid in the capsid protein corresponding to amino acid 119 of SEQ ID NO: 16 is L, and the amino acid in the capsid protein corresponding to amino acid 468 of SEQ ID NO: 16 is S; (e) the amino acid in the capsid protein corresponding to amino acid 626 of SEQ ID NO: 16 is G, and the amino acid in the capsid protein corresponding to amino acid 718 of SEQ ID NO: 16 is G; (f) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M; (g) the amino acid in the capsid protein corresponding to amino acid 505 of SEQ ID NO: 16 is R, and the amino acid in the capsid protein corresponding to amino acid 687 of SEQ ID NO: 16 is R; (h) the amino acid in the capsid protein corresponding to amino acid 346 of SEQ ID NO: 16 is A, and the amino acid in the capsid protein corresponding to amino acid 505 of SEQ ID NO: 16 is R; or the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C.

In certain embodiments, the AAV capsid protein comprises the amino acid sequence of amino acids 1-736 of SEQ ID NO: 1, 2, 3, 4, 5, 6, 7, 8, 9, 10, 11, 12, 13, 15, 16, or 17.

In certain embodiments, the third nucleotide sequence further comprises a transcriptional regulatory element operably linked to the AAV capsid protein coding sequence. In certain embodiments, the transcriptional regulatory element comprises a promoter selected from the group consisting of a constitutive promoter, an inducible promoter, or a native promoter. In certain embodiments, the promoter is selected from the group consisting of a P40 promoter, a metallothionine (MT) promoter, a mouse mammary tumor virus (MMTV) promoter, a T7 promoter, an ecdysone insect promoter, a tetracycline-repressible promoter, a tetracycline-inducible promoter, an RU486-inducible promoter, and a rapamycin-inducible promoter.

In certain embodiments, the first nucleic acid vector comprises a nucleotide sequence that is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 73 or 77.

In certain embodiments, the second nucleotide sequence comprises a sequence that is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 71, 75, 78, 80, 82, 84, 85, 86, 87, or 88.

In certain embodiments, the first nucleotide sequence comprises a sequence that is at least 85%, 86%, 87%, 88%, 89%, 90%, 91%, 92%, 93%, 94%, 95%, 96%, 97%, 98%, 99%, or 100% identical to a nucleotide sequence set forth in SEQ ID NO: 50, 51, 52, 53, 54, 55, 56, 57, 58, or 59; the second nucleotide sequence comprises a sequence that is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 71, 75, 78, 80, 82, 84, 85, 86, 87, or 88; and the third nucleotide sequence encodes an amino acid sequence that is at least 85%, 86%, 87%, 88%, 89%, 90%, 91%, 92%, 93%, 94%, 95%, 96%, 97%, 98%, 99%, or 100% identical to the amino acid sequence of amino acids 203-736, 138-736, and/or 1-736 of SEQ ID NO: 1, 2, 3, 4, 5, 6, 7, 8, 9, 10, 11, 12, 13, 15, 16, or 17.

In certain embodiments, the first nucleic acid vector comprises, from 5′ to 3′: the first nucleotide sequence; the second nucleotide sequence; and the third nucleotide sequence.

In certain embodiments, the helper virus gene is derived from a helper virus selected from the group consisting of adenovirus, herpes virus, poxvirus, cytomegalovirus, and baculovirus. In certain embodiments, the helper virus gene is an RNA gene derived from adenovirus selected from the group consisting of E1, E2, E4, and VA. In certain embodiments, the helper virus gene is a gene derived from herpes virus selected from the group consisting of UL5/8/52, ICP0, ICP4, ICP22, and UL30/UL42.

In certain embodiments, the second nucleic acid vector further comprises a transcriptional regulatory element operably linked to the helper virus gene. In certain embodiments, the transcriptional regulatory element comprises a promoter selected from the group consisting of a constitutive promoter, an inducible promoter, or a native promoter. In certain embodiments, the promoter is selected from the group consisting of an RSV LTR promoter, a CMV immediate early promoter, an SV40 promoter, a dihydrofolate reductase promoter, a cytoplasmic β-actin promoter, a phosphoglycerate kinase (PGK) promoter, a metallothionine (MT) promoter, a mouse mammary tumor virus (MMTV) promoter, a T7 promoter, an ecdysone insect promoter, a tetracycline-repressible promoter, a tetracycline-inducible promoter, an RU486-inducible promoter, and a rapamycin-inducible promoter.

In certain embodiments, the second nucleic acid vector comprises a nucleotide sequence that is at least 85%, 86%, 87%, 88%, 89%, 90%, 91%, 92%, 93%, 94%, 95%, 96%, 97%, 98%, 99%, or 100% identical to a nucleotide sequence set forth in SEQ ID NO: 60, 61, or 62.

In certain embodiments, the second nucleic acid vector comprises a nucleotide sequence that is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 63.

In another aspect, the present disclosure provides a host cell comprising a nucleic acid vector described herein, or a packaging system described herein. The present disclosure also provides a population of such host cells. In certain embodiments, the population of host cells is provided in a cell culture. In certain embodiments, the cell culture has a volume of at least 2 liters, at least 50 liters, or at least 2000 liters. In certain embodiments, the cell culture has a volume of about 2 liters to about 5000 liters. In certain embodiments, the cell culture has a volume of about 2 liters to about 4000 liters. In certain embodiments, the cell culture has a volume of about 2 liters to about 3000 liters. In certain embodiments, the cell culture has a volume of about 2 liters to about 2500 liters. In certain embodiments, the cell culture has a volume of about 2 liters to about 2000 liters. In certain embodiments, the cell culture has a volume of about 2 liters to about 1500 liters. In certain embodiments, the cell culture has a volume of about 2 liters to about 1000 liters. In certain embodiments, the cell culture has a volume of about 2 liters to about 500 liters. In certain embodiments, the cell culture has a volume of about 2 liters to about 250 liters. In certain embodiments, the cell culture has a volume of about 2 liters to about 100 liters. In certain embodiments, the cell culture has a volume of about 2 liters to about 50 liters. In certain embodiments, the cell culture has a volume of about 2 liters to about 25 liters.

In certain embodiments, the host cell is a mammalian cell. In certain embodiments, the mammalian cell is selected from the group consisting of a COS cell, a CHO cell, a BHK cell, an MDCK cell, an HEK293 cell, an HEK293T cell, an HEK293F cell, an NSO cell, a PER.C6 cell, a VERO cell, a CRL7O3O cell, an HsS78Bst cell, a HeLa cell, an NIH 3T3 cell, a HepG2 cell, an SP210 cell, an R1.1 cell, a B-W cell, an L-M cell, a BSC1 cell, a BSC40 cell, a YB/20 cell, and a BMT10 cell. In certain embodiments, the mammalian cell is an HEK293 cell.

In another aspect, the present disclosure provides a method for recombinant preparation of an rAAV, the method comprising introducing a packaging system described herein into a mammalian cell under conditions whereby the rAAV is produced.

In certain embodiments, the ratio of the first nucleic acid vector to the second nucleic acid vector or the ratio of the second nucleic acid vector to the first nucleic acid vector is selected from the group consisting of: 1:0.2, 1:0.4, 1:0.6, 1:0.8, 1:1, 1:2, 1:3, or 1:4. In certain embodiments, the ratio of the first nucleic acid vector to the second nucleic acid vector or the ratio of the second nucleic acid vector to the first nucleic acid vector is 1:2. In certain embodiments, the ratio of the first nucleic acid vector to the second nucleic acid vector or the ratio of the second nucleic acid vector to the first nucleic acid vector is from 1:0.2 to 1:1. In certain embodiments, the ratio of the first nucleic acid vector to the second nucleic acid vector or the ratio of the second nucleic acid vector to the first nucleic acid vector is 1:0.6. In certain embodiments, the ratio of the first nucleic acid vector to the second nucleic acid vector or the ratio of the second nucleic acid vector to the first nucleic acid vector is 1:0.8. In certain embodiments, the ratio of the first nucleic acid vector to the second nucleic acid vector or the ratio of the second nucleic acid vector to the first nucleic acid vector is 1:1.

In certain embodiments, the method comprises introducing from 0.1 to 4 DNA/1E6 cells of the packaging system. In certain embodiments, the method comprises introducing from 0.5 to 1 μg DNA/1E6 cells of the packaging system. In certain embodiments, the method comprises introducing 0.6, 0.7, 0.8, 0.9, or 1 μg DNA/1E6 cells of the packaging system. In certain embodiments, the method comprises introducing 0.75 μg DNA/1E6 cells of the packaging system.

In certain embodiments, the ratio of the first nucleic acid vector to the second vector nucleic acid is 1:2, 1:3, or 1:4. In certain embodiments, the ratio of the first nucleic acid vector to the second nucleic acid vector is 1:2.

In certain embodiments, the method results in an increased rAAV titer as compared to a method that comprises producing rAAV using a mammalian cell comprising: (i) a first vector comprising a nucleotide sequence encoding the AAV Rep protein and the AAV capsid protein; (ii) a second vector comprising the rAAV genome; and (iii) a third vector comprising the one or more helper virus genes.

In certain embodiments, the method results in an increased percentage of intact vector genomes as compared to a method that comprises producing rAAV using a mammalian cell comprising: (i) a first vector comprising a nucleotide sequence encoding the AAV Rep protein and the AAV capsid protein; (ii) a second vector comprising the rAAV genome; and (iii) a third vector comprising the one or more helper virus genes.

In certain embodiments, the mammalian cell is selected from the group consisting of a COS cell, a CHO cell, a BHK cell, an MDCK cell, an HEK293 cell, an HEK293T cell, an HEK293F cell, an NSO cell, a PER.C6 cell, a VERO cell, a CRL7O3O cell, an HsS78Bst cell, a HeLa cell, an NIH 3T3 cell, a HepG2 cell, an SP210 cell, an R1.1 cell, a B-W cell, an L-M cell, a BSC1 cell, a BSC40 cell, a YB/20 cell, and a BMT10 cell. In certain embodiments, the mammalian cell is an HEK293 cell.

### BRIEF DESCRIPTION OF DRAWINGS

FIGS. 1A-1C are graphs showing the viral genome (VG) productivity (FIG. 1A), capsid productivity (FIG. 1B), and percentage of intact vector genomes (FIG. 1C) obtained from small-scale rAAV production using a triple vector transfection system (1) and a dual vector transfection system (2).

FIGS. 2A-2C are graphs showing the VG productivity (FIG. 2A), capsid productivity (FIG. 2B), and percentage of intact vector genomes (FIG. 2C) obtained from small-scale rAAV production using a triple vector transfection system (1 and 3) and a dual vector transfection system (2 and 4). rAAV productivity was determined for two different rAAV gene editing vectors: a human-specific gene editing vector (1 and 2) and a mouse-specific vector (3 and 4). The various conditions are set forth in Table 3.

FIGS. 3A-3C are schematics showing rAAV dual vector transfection system design-1 (FIG. 3A), design-2 (FIG. 3B), and design-3 (FIG. 3C).

FIGS. 4A-4C are graphs showing the VG productivity (FIG. 4A), capsid productivity (FIG. 4B), and percentage of intact vector genomes (FIG. 4C) obtained from small-scale rAAV production using the dual vector transfection system design-1 (1-3), the dual vector transfection system design-2 (4-6), and a triple vector transfection system (7). The dual vector transfection system designs that were tested are as depicted in FIGS. 3A and 3B. For each dual vector transfection system design tested, transfection was performed with three different transgene vector to helper vector ratios: 1:0.5 (1 and 4), 1:1 (2 and 5), and 1:3 (3 and 6). The various transfection conditions are set forth in Table 4.

FIGS. 5A-5C are graphs showing the VG productivity (FIG. 5A), capsid productivity (FIG. 5B), and percentage of intact vector genomes (FIG. 5C; “% Full”) obtained from small-scale rAAV production using the dual vector transfection system design-1 (1), the dual vector transfection system design-2 (2), the dual vector transfection system design-3 (3), and a triple vector transfection system (4). The dual vector transfection system designs that were tested are as depicted in FIGS. 3A-3C. The various transfection conditions are set forth in Table 5.

FIGS. 6A-6C are graphs showing the VG productivity (FIG. 6A), capsid productivity (FIG. 6B), and percentage of intact vector genomes (FIG. 6C) obtained from 2 L-scale rAAV production using dual vector transfection system design-1 at various transgene vector to helper vector ratios: 1:2 (“Dual 1:2”), 1:3 (“Dual 1:3”), and 1:4 (“Dual 1:4”), and a triple vector transfection system (Triple). Six different rAAV vector genomes (1-6) were tested. Conditions 1-5 used an AAVHSC15 capsid, and condition 6 used an AAVHSC17 capsid. The various transfection conditions are set forth in Table 6.

FIGS. 7A-7C are graphs showing the VG productivity (FIG. 7A), capsid productivity (FIG. 7B), and percentage of intact vector genomes (FIG. 7C) obtained from small-scale rAAV production using dual vector transfection system design-1 (2 TFX) and a triple vector transfection system (3 TFX), utilizing an AAV2 capsid. The various transfection conditions are set forth in Table 6.

FIG. 8 is a graph showing the number of intact vector genomes obtained from rAAV production using design-1 dual plasmid systems, in each case expressed as a percentage increase over the number of intact vector genomes obtained from the corresponding triple plasmid system control. Four different rAAV vector genomes (1-4) were tested. Conditions 1-3 used an AAVHSC15 capsid, and condition 4 used an AAVHSC17 capsid. The various transfection conditions are set forth in Table 7.

FIG. 9 is a graph showing the level of capsid generation from dual vector transfection system design-1 and design-2 together with the level of capsid generation from the vector containing the Rep/Cap sequence of each respective design. The various transfection conditions are set forth in Table 8.

FIGS. 10A-10C are graphs showing the VG productivity (FIG. 10A), capsid productivity (FIG. 10B), and percentage of intact vector genomes (FIG. 10C) obtained from 50 L bioreactor rAAV production using dual vector transfection system design-1 (2 TFX) and a triple vector transfection system (3 TFX). The transfection conditions are set forth in Table 6, Condition 4, at a vector ratio of 1:2 for design-1, and the associated triple transfection control. FIGS. 10D-10F are graphs showing the percent purity (FIG. 10D), percent aggregation (FIG. 10E), and level of residual host cell protein (FIG. 10F), in purified AAV vector obtained using 2 TFX and 3 TFX systems. FIGS. 10G-10J are graphs showing the amount of residual host cell DNA (FIG. 10G), Rep/Cap (FIG. 10H), Ela (FIG. 10I), and Helper sequences (FIG. 10J) packaged in purified AAV vectors obtained using 2 TFX and 3 TFX systems. In FIGS. 10F and 10I, the horizontal dashed lines indicate the limit of detection for the assays where samples were determined to be below the limit of quantification (BLoQ). ns means not significant; * means statistically significant at p<0.05; and *** means statistically significant at p<0.001.

FIGS. 11A-11B are graphs showing the levels of phenylalanine (Phe) measured in serum of Pah^{enu2} mice administered AAV vectors obtained from Condition 5 in Table 6 at a vector ratio of 1:4 for design 1 (2 TFX), and the associated triple transfection control (3 TFX), at a dose of 1E12 VG/kg (FIG. 11A) and 1E14 VG/kg (FIG. 11B). Vehicle-only administrations were performed as control (Vehicle). FIGS. 11C-11E are graphs showing the quantification of vector genomes in the liver (FIG. 11C), transgene expression (FIG. 11D), and on-target integration (FIG. 11E) in the treated mice at six weeks post-dosing. ns means not significant.

FIGS. 12A-12C are graphs showing the VG productivity (FIG. 12A), capsid productivity (FIG. 12B), and percentage of intact vector genomes (FIG. 12C) obtained from small scale rAAV production using dual vector transfection system design-1 that tested various ratios as indicated between vectors V3 and V12, at various levels of total DNA transfected (x-axis). The PEI:DNA ratio used was 2:1.

FIGS. 13A-13C are graphs showing the VG productivity (FIG. 13A), capsid productivity (FIG. 13B), and percentage of intact vector genomes (FIG. 13C) obtained from small scale rAAV production using dual vector transfection system design-1 that tested various ratios as indicated between vectors V3 and V8, at various levels of total DNA transfected (x-axis). The PEI:DNA ratio used was 2:1.

FIGS. 14A-14C are graphs showing the VG productivity (FIG. 14A), capsid productivity (FIG. 14B) and percentage of intact vector genomes (FIG. 14C) obtained from 2 L-scale rAAV production using dual vector transfection system design-1 and the associated triple transfection control across AAV capsid serotypes AAV1, AAV2, AAV5, AAV6, AAV8, AAV9, AAVrh10 and AAVrh74.

FIG. 15 is a graph showing the VG productivity obtained from 50 L and 2000 L bioreactor rAAV production using dual vector transfection system design-1.

### DETAILED DESCRIPTION

The present disclosure provides a dual vector transfection system for the production of recombinant adeno-associated virus (rAAV). The present disclosure is based on the finding that rAAV production using the dual vector transfection approach described herein, results in superior AAV productivity over conventional triple vector transfection approaches. The specific organization of components in the dual vector transfection system described herein also results in superior AAV productivity over a prior art dual vector transfection approach.

### I. Definitions

As used herein, the term “recombinant adeno-associated virus” or “rAAV” refers to an AAV comprising a genome lacking functional rep and cap genes.

As used herein, the term “cap gene” refers to a nucleic acid sequence that encodes an AAV capsid protein.

As used herein, the term “rep gene” refers to a nucleic acid sequence that encodes AAV Rep proteins required for AAV replication (e.g., Rep78, Rep68, Rep52, and Rep40).

As used herein, the term “Rep-Cap element” refers to a nucleic acid sequence that encodes AAV Rep proteins required for AAV replication (e.g., Rep78, Rep68, Rep52, and Rep40) as well as AAV capsid proteins (e.g., VP1, VP2, and VP3).

As used herein, the term “helper virus gene” refers to a nucleic acid sequence that encodes a viral gene (e.g., an adenovirus gene, or a herpesvirus gene) that mediates AAV replication.

As used herein, the term “rAAV genome” refers to a nucleic acid molecule comprising the genome sequence of an rAAV. The skilled artisan will appreciate that where an rAAV genome comprises a transgene, the rAAV genome can be in the sense or antisense orientation relative to the direction of transcription of the transgene.

As used herein, the term “editing genome” refers to a recombinant AAV genome that is capable of integrating an editing element (e.g., one or more nucleotides or an internucleotide bond) via homologous recombination into a target locus to correct a genetic defect in a target gene. The skilled artisan will appreciate that the portion of an editing genome comprising the 5′ homology arm, editing element, and 3′ homology arm can be in the sense or antisense orientation relative to the target locus.

As used herein, the term “editing element” refers to the portion of an editing genome that when integrated at a target locus modifies the target locus. An editing element can mediate insertion, deletion, or substitution of one or more nucleotides at the target locus. As used herein, the term “target locus” refers to a region of a chromosome or an internucleotide bond (e.g., a region or an internucleotide bond of a target gene) that is modified by an editing element.

As used herein, the term “homology arm” refers to a portion of an editing genome positioned 5′ or 3′ of an editing element that is substantially identical to the genome flanking a target locus.

As used herein, the “percentage identity” between two nucleotide sequences or between two amino acid sequences is calculated by multiplying the number of matches between the pair of aligned sequences by 100, and dividing by the length of the aligned region, including internal gaps. Identity scoring only counts perfect matches and does not consider the degree of similarity of amino acids to one another. Note that only internal gaps are included in the length, not gaps at the sequence ends.

As used herein, the term “coding sequence” refers to the portion of a complementary DNA (cDNA) that encodes a polypeptide, starting at the start codon and ending at the stop codon. A gene may have one or more coding sequences due to alternative splicing, alternative translation initiation, and variation within the population. A coding sequence may be wild-type or a non-naturally occurring variant (e.g., a codon optimized variant).

As used herein, the term “transcriptional regulatory element” or “TRE” refers to a cis-acting nucleotide sequence, for example, a DNA sequence, that regulates (e.g., controls, increases, or reduces) transcription of an operably linked nucleotide sequence by an RNA polymerase to form an RNA molecule. A TRE relies on one or more trans-acting molecules, such as transcription factors, to regulate transcription. Thus, one TRE may regulate transcription in different ways when it is in contact with different trans-acting molecules, for example, when it is in different types of cells. A TRE may comprise one or more promoter elements and/or enhancer elements. A skilled artisan would appreciate that the promoter and enhancer elements in a gene may be close in location, and the term “promoter” may refer to a sequence comprising a promoter element and an enhancer element. Thus, the term “promoter” does not exclude an enhancer element in the sequence. The promoter and enhancer elements do not need to be derived from the same gene or species, and the sequence of each promoter or enhancer element may be either identical or substantially identical to the corresponding endogenous sequence in the genome.

As used herein, the term “operably linked” is used to describe the connection between a TRE and a coding sequence to be transcribed. Typically, gene expression is placed under the control of a TRE comprising one or more promoter and/or enhancer elements. The coding sequence is “operably linked” to the TRE if the transcription of the coding sequence is controlled or influenced by the TRE. The promoter and enhancer elements of the TRE may be in any orientation and/or distance from the coding sequence, as long as the desired transcriptional activity is obtained. In certain embodiments, the TRE is upstream from the coding sequence.

As used herein, the term “polyadenylation sequence” refers to a DNA sequence that when transcribed into RNA constitutes a polyadenylation signal sequence. The polyadenylation sequence can be native or exogenous. The exogenous polyadenylation sequence can be a mammalian or a viral polyadenylation sequence (e.g., an SV40 polyadenylation sequence).

As used herein, “exogenous polyadenylation sequence” refers to a polyadenylation sequence not identical or substantially identical to the endogenous polyadenylation sequence of a transgene. In certain embodiments, an exogenous polyadenylation sequence is a polyadenylation sequence of a gene different from the transgene, but within the same species (e.g., human). In certain embodiments, an exogenous polyadenylation sequence is a polyadenylation sequence of a different organism (e.g., a virus).

### II. First Nucleic Acid Vector

Conventional triple vector transfection systems for the production of rAAV typically comprise: a first vector containing sequences that encode the AAV Rep protein and the AAV capsid protein; a second vector that comprises the rAAV genome; and a third vector that comprises one or more helper virus genes. It has previously been shown that the genes encoding the AAV Rep protein, the AAV capsid protein, and the one or more helper virus genes can be cloned into the same vector as (a “Rep-Cap-Helper vector”). In such a case, double transfection of the Rep-Cap-Helper vector together with a second vector that comprises the rAAV genome (i.e., providing Rep, Cap, and Helper genes in trans to the rAAV genome) can be used to generate rAAV. See, e.g., Grimm et al. (1998) Hum. Gene Ther. 9(18): 2745-2760, the disclosure of which is incorporated by reference herein in its entirety.

In contrast to previous dual vector transfection systems, the dual vector transfection system of the present disclosure provides Rep and Cap genes in cis with the rAAV genome. Accordingly, the present disclosure provides a dual vector transfection system for the production of recombinant adeno-associated virus (rAAV), wherein the dual vector transfection system described herein generally comprises: (1) a first nucleic acid vector comprising a first nucleotide sequence encoding an AAV Rep protein, a second nucleotide sequence comprising an rAAV genome comprising a transgene, and a third nucleotide sequence encoding an AAV capsid protein; and (2) a second nucleic acid vector comprising a helper virus gene.

In certain embodiments, the first nucleic acid vector comprises from 5′ to 3′: the first nucleotide sequence encoding an AAV Rep protein, the second nucleotide sequence comprising an rAAV genome comprising a transgene, and the third nucleotide sequence encoding an AAV capsid protein. Certain aspects of the present disclosure provide that the first nucleic acid vector does not comprise a helper virus gene (e.g., a gene that encodes an AAV production helper factor).

The dual vector transfection system described herein generally involves the transfection of the first nucleic acid vector and the second nucleic acid vector into a suitable host cell to produce an AAV (e.g., an rAAV). In certain embodiments, the first nucleic acid vector and the second nucleic acid vector together provide all of the components required for AAV (e.g., rAAV) production. In certain embodiments, the first nucleic acid vector and the second nucleic acid vector, and in addition, the host cell, together provide all the components required for AAV (e.g., rAAV) production.

It has been found that the dual vector transfection system disclosed herein results in increased rAAV productivity, as compared to both conventional triple vector transfection systems and a previously described dual vector transfection system. Without being bound by any theory, Applicants believe that the provision of Rep and Cap genes in cis with the rAAV genome in the dual vector transfection system described herein, results in superior rAAV productivity, in part, because fewer empty AAV capsids are produced.

rAAV Genome

In the dual vector systems disclosed herein, the first nucleic acid vector generally comprises a nucleotide sequence comprising an rAAV genome. In certain embodiments, the rAAV genome comprises a transgene.

In certain embodiments, the transgene comprises one or more sequences encoding an RNA molecule. Suitable RNA molecules include, without limitation, miRNA, shRNA, siRNA, antisense RNA, gRNA, antagomirs, miRNA sponges, RNA aptazymes, RNA aptamers, mRNA, lncRNAs, ribozymes, and synthetic RNAs known in the art.

In certain embodiments, the transgene encodes one or more polypeptides, or a fragment thereof. Such transgenes can comprise the complete coding sequence of a polypeptide, or only a fragment of a coding sequence of a polypeptide. In certain embodiments, the transgene encodes a polypeptide that is useful to treat a disease or disorder in a subject. Suitable polypeptides include, without limitation, 3-globin, hemoglobin, tissue plasminogen activator, and coagulation factors; colony stimulating factors (CSF); interleukins, such as IL-1, IL-2, IL-3, IL-4, IL-5, IL-6, IL-7, IL-8, IL-9, etc.; growth factors, such as keratinocyte growth factor (KGF), stem cell factor (SCF), fibroblast growth factor (FGF, such as basic FGF and acidic FGF), hepatocyte growth factor (HGF), insulin-like growth factors (IGFs), bone morphogenetic protein (BMP), epidermal growth factor (EGF), growth differentiation factor-9 (GDF-9), hepatoma derived growth factor (HDGF), myostatin (GDF-8), nerve growth factor (NGF), neurotrophins, platelet-derived growth factor (PDGF), thrombopoietin (TPO), transforming growth factor alpha (TGF-a), transforming growth factor beta (TGF-β), and the like; soluble receptors, such as soluble TNF-α receptors, soluble interleukin receptors (e.g., soluble IL-1 receptors and soluble type II IL-1 receptors), soluble γ/Δ T cell receptors, ligand-binding fragments of a soluble receptor, and the like; enzymes, such as a-glucosidase, imiglucerase, 3-glucocerebrosidase, and alglucerase; enzyme activators, such as tissue plasminogen activator; chemokines, such as IP-10, monokine induced by interferon-gamma (Mig), Groα/IL-8, RANTES, MIP-1a, MIP-1β, MCP-1, PF-4, and the like; angiogenic agents, such as vascular endothelial growth factors (VEGFs, e.g., VEGF121, VEGF165, VEGF-C, VEGF-2), glioma-derived growth factor, angiogenin, angiogenin-2; and the like; anti-angiogenic agents, such as a soluble VEGF receptor; protein vaccine; neuroactive peptides, such as nerve growth factor (NGF), bradykinin, cholecystokinin, gastrin, secretin, oxytocin, gonadotropin-releasing hormone, beta-endorphin, enkephalin, substance P, somatostatin, prolactin, galanin, growth hormone-releasing hormone, bombesin, dynorphin, warfarin, neurotensin, motilin, thyrotropin, neuropeptide Y, luteinizing hormone, calcitonin, insulin, glucagons, vasopressin, angiotensin II, thyrotropin-releasing hormone, vasoactive intestinal peptide, a sleep peptide, and the like; thrombolytic agents; atrial natriuretic peptide; relaxin; glial fibrillary acidic protein; follicle stimulating hormone (FSH); human alpha-1 antitrypsin; leukemia inhibitory factor (LIF); tissue factors; macrophage activating factors; tumor necrosis factor (TNF); neutrophil chemotactic factor (NCF); tissue inhibitors of metalloproteinases; vasoactive intestinal peptide; angiogenin; angiotropin; fibrin; hirudin; IL-1 receptor antagonists; ciliary neurotrophic factor (CNTF); brain-derived neurotrophic factor (BDNF); neurotrophins 3 and 4/5 (NT-3 and -4/5); glial cell derived neurotrophic factor (GDNF); aromatic amino acid decarboxylase (AADC); Factor VIII, Factor IX, Factor X; dystrophin or mini-dystrophin; lysosomal acid lipase; phenylalanine hydroxylase (PAH); glycogen storage disease-related enzymes, such as glucose-6-phosphatase, acid maltase, glycogen debranching enzyme, muscle glycogen phosphorylase, liver glycogen phosphorylase, muscle phosphofructokinase, phosphorylase kinase, glucose transporter, aldolase A, β-enolase, glycogen synthase; lysosomal enzymes, such as iduronate-2-sulfatase (12S), and arylsulfatase A; and mitochondrial proteins, such as frataxin.

In certain embodiments, the transgene encodes a protein that may be defective in one or more lysosomal storage diseases. Suitable proteins include, without limitation, α-sialidase, cathepsin A, α-mannosidase, β-mannosidase, glycosylasparaginase, α-fucosidase, α-N-acetylglucosaminidase, β-galactosidase, β-hexosaminidase α-subunit, β-hexosaminidase β-subunit, GM2 activator protein, glucocerebrosidase, Saposin C, Arylsulfatase A, Saposin B, formyl-glycine generating enzyme, β-galactosylceramidase, α-galactosidase A, iduronate sulfatase, α-iduronidase, heparan N-sulfatase, acetyl-CoA transferase, N-acetyl glucosaminidase, β-glucuronidase, N-acetyl glucosamine 6-sulfatase, N-acetylgalactosamine 4-sulfatase, galactose 6-sulfatase, hyaluronidase, α-glucosidase, acid sphingomyelinase, acid ceramidase, acid lipase, cathepsin K, tripeptidyl peptidase, palmitoyl-protein thioesterase, cystinosin, sialin, UDP-N-acetylglucosamine, phosphotransferase γ-subunit, mucolipin-1, LAMP-2, NPC1, CLN3, CLN 6, CLN 8, LYST, MYOV, RAB27A, melanophilin, and AP3 β-subunit.

In certain embodiments, the transgene encodes an antibody or a fragment thereof (e.g., a Fab, scFv, or full-length antibody). Suitable antibodies include, without limitation, muromonab-cd3, efalizumab, tositumomab, daclizumab, nebacumab, catumaxomab, edrecolomab, abciximab, rituximab, basiliximab, palivizumab, infliximab, trastuzumab, adalimumab, ibritumomab tiuxetan, omalizumab, cetuximab, bevacizumab, natalizumab, panitumumab, ranibizumab, eculizumab, certolizumab, ustekinumab, canakinumab, golimumab, ofatumumab, tocilizumab, denosumab, belimumab, ipilimumab, brentuximab vedotin, pertuzumab, raxibacumab, obinutuzumab, alemtuzumab, siltuximab, ramucirumab, vedolizumab, blinatumomab, nivolumab, pembrolizumab, idarucizumab, necitumumab, dinutuximab, secukinumab, mepolizumab, alirocumab, evolocumab, daratumumab, elotuzumab, ixekizumab, reslizumab, olaratumab, bezlotoxumab, atezolizumab, obiltoxaximab, inotuzumab ozogamicin, brodalumab, guselkumab, dupilumab, sarilumab, avelumab, ocrelizumab, emicizumab, benralizumab, gemtuzumab ozogamicin, durvalumab, burosumab, erenumab, galcanezumab, lanadelumab, mogamulizumab, tildrakizumab, cemiplimab, fremanezumab, ravulizumab, emapalumab, ibalizumab, moxetumomab, caplacizumab, romosozumab, risankizumab, polatuzumab, eptinezumab, leronlimab, sacituzumab, brolucizumab, isatuximab, and teprotumumab.

In certain embodiments, the transgene encodes a nuclease. Suitable nucleases include, without limitation, zinc fingers nucleases (ZFN) (see, e.g., Porteus, and Baltimore (2003) Science 300: 763; Miller et al. (2007) Nat. Biotechnol. 25:778-785; Sander et al. (2011) Nature Methods 8:67-69; and Wood et al. (2011) Science 333:307, each of which is hereby incorporated by reference in its entirety), transcription activator-like effectors nucleases (TALEN) (see, e.g., Wood et al. (2011) Science 333:307; Boch et al. (2009) Science 326:1509-1512; Moscou and Bogdanove (2009) Science 326:1501; Christian et al. (2010) Genetics 186:757-761; Miller et al. (2011) Nat. Biotechnol. 29:143-148; Zhang et al. (2011) Nat. Biotechnol. 29:149-153; and Reyon et al. (2012) Nat. Biotechnol. 30(5): 460-465, each of which is hereby incorporated by reference in its entirety), homing endonucleases, meganucleases (see, e.g., U.S. Patent Publication No. US 2014/0121115, which is hereby incorporated by reference in its entirety), and RNA-guided nucleases (see, e.g., Makarova et al. (2018) The CRISPR Journal 1(5): 325-336; and Adli (2018) Nat. Communications 9:1911, each of which is hereby incorporated by reference in its entirety).

In certain embodiments, the transgene encodes an RNA-guided nuclease. Suitable RNA-guided nucleases include, without limitation, Class I and Class II clustered regularly interspaced short palindromic repeats (CRISPR)-associated nucleases. Class I is divided into types I, III, and IV, and includes, without limitation, type I (Cas3), type I-A (Cas8a, Cas5), type I-B (Cas8b), type I-C(Cas8c), type I-D (Cas10d), type I-E (Cse1, Cse2), type I-F (Csy1, Csy2, Csy3), type I-U (GSU0054), type III (Cas10), type III-A (Csm2), type III-B (Cmr5), type III-C (Csx10 or Csx11), type III-D (Csx10), and type IV (Csfl). Class II is divided into types II, V, and VI, and includes, without limitation, type II (Cas9), type II-A (Csn2), type II-B (Cas4), type V (Cpf1, C2c1, C2c3), and type VI (Cas13a, Cas13b, Cas13c). RNA-guided nucleases also include naturally-occurring Class II CRISPR nucleases such as Cas9 (Type II) or Cas12a/Cpf1 (Type V), as well as other nucleases derived or obtained therefrom. Exemplary Cas9 nucleases that may be used in the present invention include, but are not limited to, S. pyogenes Cas9 (SpCas9), S. aureus Cas9 (SaCas9), N. meningitidis Cas9 (NmCas9), C. jejuni Cas9 (CjCas9), and Geobacillus Cas9 (GeoCas9).

In certain embodiments, the transgene encodes one or more reporter sequences, which upon expression produce a detectable signal. Such reporter sequences include, without limitation, DNA sequences encoding β-lactamase, β-galactosidase (LacZ), alkaline phosphatase, thymidine kinase, green fluorescent protein (GFP), red fluorescent protein (RFP), chloramphenicol acetyltransferase (CAT), luciferase, membrane bound proteins, including, for example, CD2, CD4, CD8, the influenza hemagglutinin protein, and others well known in the art, to which high affinity antibodies directed thereto exist or can be produced by conventional means, and fusion proteins comprising a membrane bound protein appropriately fused to an antigen tag domain from, among others, hemagglutinin or Myc.

In certain embodiments, the rAAV genome comprises a transcriptional regulatory element (TRE) operably linked to the transgene, to control expression of an RNA or polypeptide encoded by the transgene. In certain embodiments, the TRE comprises a constitutive promoter. In certain embodiments, the TRE can be active in any mammalian cell (e.g., any human cell). In certain embodiments, the TRE is active in a broad range of human cells. Such TREs may comprise constitutive promoter and/or enhancer elements, including any of those described herein, and any of those known to one of skill in the art. In certain embodiments, the TRE comprises an inducible promoter. In certain embodiments, the TRE may be a tissue-specific TRE, i.e., it is active in specific tissue(s) and/or organ(s). A tissue-specific TRE comprises one or more tissue-specific promoter and/or enhancer elements, and optionally one or more constitutive promoter and/or enhancer elements. A skilled artisan would appreciate that tissue-specific promoter and/or enhancer elements can be isolated from genes specifically expressed in the tissue by methods well known in the art.

Suitable promoters include, e.g., cytomegalovirus promoter (CMV) (Stinski et al. (1985) Journal of Virology 55(2): 431-441), CMV early enhancer/chicken β-actin (CBA) promoter/rabbit β-globin intron (CAG) (Miyazaki et al. (1989) Gene 79(2): 269-277), CB^{SB} (Jacobson et al. (2006) Molecular Therapy 13(6): 1074-1084), human elongation factor 1α promoter (EF1α) (Kim et al. (1990) Gene 91 (2): 217-223), human phosphoglycerate kinase promoter (PGK) (Singer-Sam et al. (1984) Gene 32(3): 409-417), mitochondrial heavy-strand promoter (Lodeiro et al. (2012) PNAS 109(17): 6513-6518), ubiquitin promoter (Wulff et al. (1990) FEBS Letters 261: 101-105). In certain embodiments, the TRE comprises a cytomegalovirus (CMV) promoter/enhancer (e.g., comprising a nucleotide sequence at least 90%, 91%, 92%, 93%, 94%, 95%, 96%, 97%, 98%, 99%, or 100% identical to SEQ ID NO: 18 or 19), an SV40 promoter, a chicken beta actin (CBA) promoter (e.g., comprising a nucleotide sequence at least 90%, 91%, 92%, 93%, 94%, 95%, 96%, 97%, 98%, 99%, or 100% identical to SEQ ID NO: 20 or 21), a smCBA promoter (e.g., comprising a nucleotide sequence at least 90%, 91%, 92%, 93%, 94%, 95%, 96%, 97%, 98%, 99%, or 100% identical to SEQ ID NO: 22), a human elongation factor 1 alpha (EF1α) promoter (e.g., comprising a nucleotide sequence at least 90%, 91%, 92%, 93%, 94%, 95%, 96%, 97%, 98%, 99%, or 100% identical to SEQ ID NO: 23), a minute virus of mouse (MVM) intron which comprises transcription factor binding sites (e.g., comprising a nucleotide sequence at least 90%, 91%, 92%, 93%, 94%, 95%, 96%, 97%, 98%, 99%, or 100% identical to SEQ ID NO: 24 or 25), a human phosphoglycerate kinase (PGK1) promoter, a human ubiquitin C (Ubc) promoter, a human beta actin promoter, a human neuron-specific enolase (ENO2) promoter, a human beta-glucuronidase (GUSB) promoter, a rabbit beta-globin element (e.g., comprising a nucleotide sequence at least 90%, 91%, 92%, 93%, 94%, 95%, 96%, 97%, 98%, 99%, or 100% identical to SEQ ID NO: 26 or 27), a human calmodulin 1 (CALM1) promoter (e.g., comprising a nucleotide sequence at least 90%, 91%, 92%, 93%, 94%, 95%, 96%, 97%, 98%, 99%, or 100% identical to SEQ ID NO: 28), a human ApoE/C-I hepatic control region (HCR1) (e.g., comprising a nucleotide sequence at least 90%, 91%, 92%, 93%, 94%, 95%, 96%, 97%, 98%, 99%, or 100% identical to SEQ ID NO: 29), a human al-antitrypsin (hAAT) promoter (e.g., comprising a nucleotide sequence at least 90%, 91%, 92%, 93%, 94%, 95%, 96%, 97%, 98%, 99%, or 100% identical to SEQ ID NO: 30, 31, or 32), an extended HCR1 (e.g., comprising a nucleotide sequence at least 90%, 91%, 92%, 93%, 94%, 95%, 96%, 97%, 98%, 99%, or 100% identical to SEQ ID NO: 33), an HS-CRM8 element of an hAAT promoter (e.g., comprising a nucleotide sequence at least 90%, 91%, 92%, 93%, 94%, 95%, 96%, 97%, 98%, 99%, or 100% identical to SEQ ID NO: 34), a human transthyretin (TTR) promoter (e.g., comprising a nucleotide sequence at least 90%, 91%, 92%, 93%, 94%, 95%, 96%, 97%, 98%, 99%, or 100% identical to SEQ ID NO: 35), and/or a human Methyl-CpG Binding Protein 2 (MeCP2) promoter. Any of the TREs described herein can be combined in any order to drive efficient transcription. For example, an rAAV genome may comprise a TRE comprising a CMV enhancer, a CBA promoter, and the splice acceptor from exon 3 of the rabbit beta-globin gene, collectively called a CAG promoter (e.g., comprising a nucleotide sequence at least 90%, 91%, 92%, 93%, 94%, 95%, 96%, 97%, 98%, 99%, or 100% identical to SEQ ID NO: 36). For example, an rAAV genome may comprise a TRE comprising a hybrid of CMV enhancer and CBA promoter followed by a splice donor and splice acceptor, collectively called a CASI promoter region (e.g., comprising a nucleotide sequence at least 90%, 91%, 92%, 93%, 94%, 95%, 96%, 97%, 98%, 99%, or 100% identical to SEQ ID NO: 37). For example, an rAAV genome may comprise a TRE comprising an HCR1 and hAAT promoter (also referred to as an LP1 promoter, e.g., comprising a nucleotide sequence at least 90%, 91%, 92%, 93%, 94%, 95%, 96%, 97%, 98%, 99%, or 100% identical to SEQ ID NO: 38).

In certain embodiments, the TRE is brain-specific (e.g., neuron-specific, glial cell-specific, astrocyte-specific, oligodendrocyte-specific, microglia-specific and/or central nervous system-specific). Exemplary brain-specific TREs may comprise one or more elements from, without limitation, human glial fibrillary acidic protein (GFAP) promoter, human synapsin 1 (SYN1) promoter, human synapsin 2 (SYN2) promoter, human metallothionein 3 (MT3) promoter, and/or human proteolipid protein 1 (PLP1) promoter. More brain-specific promoter elements are disclosed in WO 2016/100575A1, which is incorporated by reference herein in its entirety.

In certain embodiments, the native promoter for the transgene may be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In certain embodiments, the rAAV genome comprises an editing genome. Editing genomes can be used to edit the genome of a cell by homologous recombination of the editing genome with a genomic region surrounding a target locus in the cell. In certain embodiments, the editing genome is designed to correct a genetic defect in a gene by homologous recombination. Editing genomes generally comprise: (i) an editing element for editing a target locus in a target gene; (ii) a 5′ homology arm nucleotide sequence 5′ of the editing element having homology to a first genomic region 5′ to the target locus; and (iii) a 3′ homology arm nucleotide sequence 3′ of the editing element having homology to a second genomic region 3′ to the target locus, wherein the portion of the editing genome comprising the 5′ homology arm, editing element, and 3′ homology arm can be in the sense or antisense orientation relative to the target locus. Suitable target genes for editing using an editing genome include, without limitation, phenylalanine hydroxylase (PAH), cystic fibrosis conductance transmembrane regulator (CFTR), beta hemoglobin (HBB), oculocutaneous albinism II (OCA2), Huntingtin (HTT), dystrophia myotonica-protein kinase (DMPK), low-density lipoprotein receptor (LDLR), apolipoprotein B (APOB), neurofibromin 1 (NF1), polycystic kidney disease 1 (PKD1), polycystic kidney disease 2 (PKD2), coagulation factor VIII (F8), dystrophin (DMD), phosphate-regulating endopeptidase homologue, X-linked (PHEX), methyl-CpG-binding protein 2 (MECP2), and ubiquitin-specific peptidase 9Y, Y-linked (USP9Y).

In certain embodiments, the rAAV genomes disclosed herein further comprise a transcription terminator (e.g., a polyadenylation sequence). In certain embodiments, the transcription terminator is 3′ to the transgene. The transcription terminator may be any sequence that effectively terminates transcription, and a skilled artisan would appreciate that such sequences can be isolated from any genes that are expressed in the cell in which transcription of the at least a portion of an antibody coding sequence is desired. In certain embodiments, the transcription terminator comprises a polyadenylation sequence. In certain embodiments, the polyadenylation sequence is identical or substantially identical to the endogenous polyadenylation sequence of an immunoglobulin gene. In certain embodiments, the polyadenylation sequence is an exogenous polyadenylation sequence. In certain embodiments, the polyadenylation sequence is an SV40 polyadenylation sequence (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 65, 68, or 69, or a nucleotide sequence complementary thereto). In certain embodiments, the polyadenylation sequence comprises the nucleotide sequence set forth in SEQ ID NO: 65. In certain embodiments, the polyadenylation sequence consists of the nucleotide sequence set forth in SEQ ID NO: 65. In certain embodiments, the polyadenylation sequence is a bovine growth hormone (BGH) polyadenylation sequence (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 67, or a nucleotide sequence complementary thereto). In certain embodiments, the polyadenylation sequence comprises the nucleotide sequence set forth in SEQ ID NO: 67. In certain embodiments, the polyadenylation sequence consists of the nucleotide sequence set forth in SEQ ID NO: 67.

In certain embodiments, an rAAV genome comprises a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence set forth in SEQ ID NO: 71, 85, 86, 87, or 88. In certain embodiments, the editing element comprises the nucleotide sequence set forth in SEQ ID NO: 71, 85, 86, 87, or 88. In certain embodiments, the editing element consists of the nucleotide sequence set forth in SEQ ID NO: 71, 85, 86, 87, or 88.

In certain embodiments, the rAAV genomes disclosed herein further comprise a 5′ inverted terminal repeat (5′ ITR) nucleotide sequence 5′ of the TRE, and a 3′ inverted terminal repeat (3′ ITR) nucleotide sequence 3′ of the polyadenylation sequence associated with an antibody light chain coding sequence. ITR sequences from any AAV serotype or variant thereof can be used in the rAAV genomes disclosed herein. The 5′ and 3′ ITR can be from an AAV of the same serotype or from AAVs of different serotypes. Exemplary ITRs for use in the rAAV genomes disclosed herein are set forth in SEQ ID NOs: 39, 40, 41, 42, 43, and 44, herein.

In certain embodiments, the 5′ ITR or 3′ ITR is from AAV2. In certain embodiments, both the 5′ ITR and the 3′ ITR are from AAV2. In certain embodiments, the 5′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 39, or the 3′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 40. In certain embodiments, the 5′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 39, and the 3′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 40. In certain embodiments, the rAAV genome comprises a 5′ ITR nucleotide sequence having the sequence of SEQ ID NO: 39, and a 3′ ITR nucleotide sequence having the sequence of SEQ ID NO: 40.

In certain embodiments, the 5′ ITR or 3′ ITR are from AAV5. In certain embodiments, both the 5′ ITR and 3′ ITR are from AAV5. In certain embodiments, the 5′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 42, or the 3′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 43. In certain embodiments, the 5′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 42, and the 3′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 43. In certain embodiments, the rAAV genome comprises a 5′ ITR nucleotide sequence having the sequence of SEQ ID NO: 42, and a 3′ ITR nucleotide sequence having the sequence of SEQ ID NO: 43.

In certain embodiments, the 5′ ITR nucleotide sequence and the 3′ ITR nucleotide sequence are substantially complementary to each other (e.g., are complementary to each other except for mismatch at 1, 2, 3, 4, or 5 nucleotide positions in the 5′ or 3′ ITR).

In certain embodiments, the 5′ ITR or the 3′ ITR is modified to reduce or abolish resolution by Rep protein (“non-resolvable ITR”). In certain embodiments, the non-resolvable ITR comprises an insertion, deletion, or substitution in the nucleotide sequence of the terminal resolution site. Such modification allows formation of a self-complementary, double-stranded DNA genome of the AAV after the rAAV genome is replicated in an infected cell. Exemplary non-resolvable ITR sequences are known in the art (see, e.g., those provided in U.S. Pat. Nos. 7,790,154 and 9,783,824, which are incorporated by reference herein in their entirety). In certain embodiments, the 5′ ITR comprises a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 41. In certain embodiments, the 5′ ITR consists of a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 41. In certain embodiments, the 5′ ITR consists of the nucleotide sequence set forth in SEQ ID NO: 41. In certain embodiments, the 3′ ITR comprises a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 44. In certain embodiments, the 5′ ITR consists of a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 44. In certain embodiments, the 3′ ITR consists of the nucleotide sequence set forth in SEQ ID NO: 44. In certain embodiments, the 5′ ITR consists of the nucleotide sequence set forth in SEQ ID NO: 41, and the 3′ ITR consists of the nucleotide sequence set forth in SEQ ID NO: 44. In certain embodiments, the 5′ ITR consists of the nucleotide sequence set forth in SEQ ID NO: 41, and the 3′ ITR consists of the nucleotide sequence set forth in SEQ ID NO: 44.

In certain embodiments, the 5′ ITR is flanked by an additional nucleotide sequence derived from a wild-type AAV2 genomic sequence. In certain embodiments, the 5′ ITR is flanked by an additional 46 bp sequence derived from a wild-type AAV2 sequence that is adjacent to a wild-type AAV2 ITR in an AAV2 genome. In certain embodiments, the additional 46 bp sequence is 3′ to the 5′ ITR in the rAAV genome. In certain embodiments, the 46 bp sequence consists of the nucleotide sequence set forth in SEQ ID NO: 45.

In certain embodiments, the 3′ ITR is flanked by an additional nucleotide sequence derived from a wild-type AAV2 genomic sequence. In certain embodiments, the 3′ ITR is flanked by an additional 37 bp sequence derived from a wild-type AAV2 sequence that is adjacent to a wild-type AAV2 ITR in an AAV2 genome. See, e.g., Savy et al., Human Gene Therapy Methods (2017) 28(5): 277-289 (which is hereby incorporated by reference herein in its entirety). In certain embodiments, the additional 37 bp sequence is 5′ to the 3′ ITR in the rAAV genome. In certain embodiments, the 37 bp sequence consists of the nucleotide sequence set forth in SEQ ID NO: 46.

In certain embodiments, an rAAV genome comprises a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence set forth in SEQ ID NO: 75, 78, 80, 82, or 84. In certain embodiments, the editing element comprises the nucleotide sequence set forth in SEQ ID NO: 75, 78, 80, 82, or 84. In certain embodiments, the editing element consists of the nucleotide sequence set forth in SEQ ID NO: 75, 78, 80, 82, or 84.

### AAV Rep Protein

The present disclosure provides a first nucleic acid vector comprising a Rep protein coding sequence or a coding sequence of a functional variant thereof. Expression of the AAV Rep gene is controlled through the use of two promoters and alternative splicing, and results in four Rep proteins, Rep78, Rep68, Rep52, and Rep40. The Rep proteins are involved in AAV genome replication and packaging of the viral genome. Expression of Rep proteins is controlled by the p5 and p19 promoters. The p5 promoter drives expression of the alternative splice variants Rep78 and Rep68. The p19 promoter drives expression of the alternative splice variants Rep52 and Rep40. Accordingly, the first nucleic acid vector can comprise a nucleotide sequence encoding one or more Rep proteins or functional variants thereof.

The one or more Rep proteins may be derived from AAV2. An exemplary AAV2 genome sequence can be found via NCBI Reference Sequence NC_001401.2. According to the NCBI Reference Sequence, Rep68 is encoded by nucleotides 321 to 2252; Rep78 is encoded by nucleotides 321 to 2186; Rep40 is encoded by nucleotides 993 to 2252; and Rep52 is encoded by nucleotides 993 to 2186.

In certain embodiments, the present disclosure provides a nucleic acid comprising a nucleotide sequence encoding Rep78, wherein the nucleotide sequence encoding for Rep78 comprises a sequence having at least 85%, 86%, 87%, 88%, 89%, 90%, 91%, 92%, 93%, 94%, 95%, 96%, 97%, 98%, or 99% sequence identity to the sequence set forth in SEQ ID NO: 50. In certain embodiments, the nucleotide sequence encoding for Rep78 comprises or consists of the sequence set forth in SEQ ID NO: 50. In certain embodiments, the nucleic acid comprising a nucleotide sequence encoding Rep78 comprises a transcriptional regulatory element operably linked to the nucleotide sequence encoding Rep78. In certain embodiments, the transcriptional regulatory element operably linked to the nucleotide sequence encoding Rep78 comprises a sequence having at least 85%, 86%, 87%, 88%, 89%, 90%, 91%, 92%, 93%, 94%, 95%, 96%, 97%, 98%, or 99% sequence identity to the sequence set forth in SEQ ID NO: 47. In certain embodiments, the transcriptional regulatory element operably linked to the nucleotide sequence encoding Rep78 comprises or consists of the sequence set forth in SEQ ID NO: 47. In certain embodiments, the nucleic acid comprising a nucleotide sequence encoding Rep78 comprises a sequence having at least 85%, 86%, 87%, 88%, 89%, 90%, 91%, 92%, 93%, 94%, 95%, 96%, 97%, 98%, or 99% sequence identity to the sequence set forth in SEQ ID NO: 51. In certain embodiments, the nucleic acid comprising a nucleotide sequence encoding Rep78 comprises or consists of the sequence set forth in SEQ ID NO: 51. In certain embodiments, the present disclosure provides a nucleic acid comprising a nucleotide sequence corresponding to the sequence encoding Rep78 as described for AAV2, in a different adenovirus serotype.

In certain embodiments, the present disclosure provides a nucleic acid comprising a nucleotide sequence encoding Rep68, wherein the nucleotide sequence encoding for Rep68 comprises a sequence having at least 85%, 86%, 87%, 88%, 89%, 90%, 91%, 92%, 93%, 94%, 95%, 96%, 97%, 98%, or 99% sequence identity to the sequence set forth in SEQ ID NO: 52. In certain embodiments, the nucleotide sequence encoding for Rep68 comprises or consists of the sequence set forth in SEQ ID NO: 52. In certain embodiments, the nucleic acid comprising a nucleotide sequence encoding Rep68 comprises a transcriptional regulatory element operably linked to the nucleotide sequence encoding Rep68. In certain embodiments, the transcriptional regulatory element operably linked to the nucleotide sequence encoding Rep68 comprises a sequence having at least 85%, 86%, 87%, 88%, 89%, 90%, 91%, 92%, 93%, 94%, 95%, 96%, 97%, 98%, or 99% sequence identity to the sequence set forth in SEQ ID NO: 47. In certain embodiments, the transcriptional regulatory element operably linked to the nucleotide sequence encoding Rep68 comprises or consists of the sequence set forth in SEQ ID NO: 47. In certain embodiments, the nucleic acid comprising a nucleotide sequence encoding Rep68 comprises a sequence having at least 85%, 86%, 87%, 88%, 89%, 90%, 91%, 92%, 93%, 94%, 95%, 96%, 97%, 98%, or 99% sequence identity to the sequence set forth in SEQ ID NO: 53. In certain embodiments, the nucleic acid comprising a nucleotide sequence encoding Rep68 comprises or consists of the sequence set forth in SEQ ID NO: 53. In certain embodiments, the present disclosure provides a nucleic acid comprising a nucleotide sequence corresponding to the sequence encoding Rep68 as described for AAV2, in a different adenovirus serotype.

In certain embodiments, the present disclosure provides a nucleic acid comprising a nucleotide sequence encoding Rep40, wherein the nucleotide sequence encoding for Rep40 comprises a sequence having at least 85%, 86%, 87%, 88%, 89%, 90%, 91%, 92%, 93%, 94%, 95%, 96%, 97%, 98%, or 99% sequence identity to the sequence set forth in SEQ ID NO: 54. In certain embodiments, the nucleotide sequence encoding for Rep40 comprises or consists of the sequence set forth in SEQ ID NO: 54. In certain embodiments, the nucleic acid comprising a nucleotide sequence encoding Rep40 comprises a transcriptional regulatory element operably linked to the nucleotide sequence encoding Rep40. In certain embodiments, the transcriptional regulatory element operably linked to the nucleotide sequence encoding Rep40 comprises a sequence having at least 85%, 86%, 87%, 88%, 89%, 90%, 91%, 92%, 93%, 94%, 95%, 96%, 97%, 98%, or 99% sequence identity to the sequence set forth in SEQ ID NO: 48. In certain embodiments, the transcriptional regulatory element operably linked to the nucleotide sequence encoding Rep40 comprises or consists of the sequence set forth in SEQ ID NO: 48. In certain embodiments, the nucleic acid comprising a nucleotide sequence encoding Rep40 comprises a sequence having at least 85%, 86%, 87%, 88%, 89%, 90%, 91%, 92%, 93%, 94%, 95%, 96%, 97%, 98%, or 99% sequence identity to the sequence set forth in SEQ ID NO: 55. In certain embodiments, the nucleic acid comprising a nucleotide sequence encoding Rep40 comprises or consists of the sequence set forth in SEQ ID NO: 55. In certain embodiments, the present disclosure provides a nucleic acid comprising a nucleotide sequence corresponding to the sequence encoding Rep40 as described for AAV2, in a different adenovirus serotype.

In certain embodiments, the present disclosure provides a nucleic acid comprising a nucleotide sequence encoding Rep52, wherein the nucleotide sequence encoding for Rep52 comprises a sequence having at least 85%, 86%, 87%, 88%, 89%, 90%, 91%, 92%, 93%, 94%, 95%, 96%, 97%, 98%, or 99% sequence identity to the sequence set forth in SEQ ID NO: 56. In certain embodiments, the nucleotide sequence encoding for Rep52 comprises or consists of the sequence set forth in SEQ ID NO: 56. In certain embodiments, the nucleic acid comprising a nucleotide sequence encoding Rep52 comprises a transcriptional regulatory element operably linked to the nucleotide sequence encoding Rep52. In certain embodiments, the transcriptional regulatory element operably linked to the nucleotide sequence encoding Rep52 comprises a sequence having at least 85%, 86%, 87%, 88%, 89%, 90%, 91%, 92%, 93%, 94%, 95%, 96%, 97%, 98%, or 99% sequence identity to the sequence set forth in SEQ ID NO: 48. In certain embodiments, the transcriptional regulatory element operably linked to the nucleotide sequence encoding Rep52 comprises or consists of the sequence set forth in SEQ ID NO: 48. In certain embodiments, the nucleic acid comprising a nucleotide sequence encoding Rep52 comprises a sequence having at least 85%, 86%, 87%, 88%, 89%, 90%, 91%, 92%, 93%, 94%, 95%, 96%, 97%, 98%, or 99% sequence identity to the sequence set forth in SEQ ID NO: 57. In certain embodiments, the nucleic acid comprising a nucleotide sequence encoding Rep52 comprises or consists of the sequence set forth in SEQ ID NO: 57. In certain embodiments, the present disclosure provides a nucleic acid comprising a nucleotide sequence corresponding to the sequence encoding Rep52 as described for AAV2, in a different adenovirus serotype.

In certain embodiments, the present disclosure provides a nucleic acid comprising a nucleotide sequence encoding Rep78, Rep68, Rep40, and Rep52, wherein the nucleotide sequence encoding for Rep78, Rep68, Rep40, and Rep52 comprises a sequence having at least 85%, 86%, 87%, 88%, 89%, 90%, 91%, 92%, 93%, 94%, 95%, 96%, 97%, 98%, or 99% sequence identity to the sequence set forth in SEQ ID NO: 58. In certain embodiments, the nucleotide sequence encoding for Rep78, Rep68, Rep40, and Rep52 comprises or consists of the sequence set forth in SEQ ID NO: 58. In certain embodiments, the nucleic acid comprising a nucleotide sequence encoding Rep78, Rep68, Rep40, and Rep52 comprises one or more transcriptional regulatory elements that may be operably linked to each of the nucleotide sequences encoding Rep78, Rep68, Rep40, and Rep52. In certain embodiments, the nucleic acid comprising a nucleotide sequence encoding Rep78, Rep68, Rep40, and Rep52 comprises a sequence having at least 85%, 86%, 87%, 88%, 89%, 90%, 91%, 92%, 93%, 94%, 95%, 96%, 97%, 98%, or 99% sequence identity to the sequence set forth in SEQ ID NO: 59. In certain embodiments, the nucleic acid comprising a nucleotide sequence encoding Rep78, Rep68, Rep40, and Rep52 comprises or consists of the sequence set forth in SEQ ID NO: 59.

### AAV Capsid Protein

The present disclosure provides a first nucleic acid vector comprising a nucleotide sequence comprising an AAV capsid protein coding sequence. The first nucleic acid vector can comprise a nucleotide sequence encoding an AAV capsid protein from any AAV capsid known in the art, including natural AAV isolates and variants thereof.

AAV capsid proteins include VP1, VP2, and VP3 capsid proteins. VP1, VP2, and/or VP3 capsid proteins assemble into a capsid that surrounds the rAAV genome. In certain embodiments, assembly of the capsid proteins is facilitated by the assembly-activating protein (AAP). Capsids of certain AAV serotypes require the role of AAP in transporting the capsid proteins to the nucleolus for assembly. For example, AAV1, AAV2, AAV3, AAV6, AAV7, AAV8, AAV9, AAV10, and AAV12 require AAP to form capsids, while capsids of AAV4, AAV5, and AAV11 can assemble without AAP. See, e.g., Earley et al. (2017) J. Virol. 91(3): e01980-16.

Different AAV serotypes or variants thereof comprise AAV capsid proteins having different amino acid sequences. Suitable AAV capsid proteins include, without limitation, a capsid protein from AAV1, AAV2, AAV3, AAV4, AAV5, AAV6, AAV7, AAV8, AAV9, AAV10, AAV11, AAV12, AAV13, AAV-DJ, AAV-LK03, NP59, VOY101, VOY201, VOY701, VOY801, VOY1101, AAVPHP.N, AAVPHP.A, AAVPHP.B, PHP.B2, PHP.B3, G2A3, G2B4, G2B5, PHP.S, AAVrh10, AAVRh32.33, AAVrh74, AAVHSC1, AAVHSC2, AAVHSC3, AAVHSC4, AAVHSC5, AAVHSC6, AAVHSC7, AAVHSC8, AAVHSC9, AAVHSC10, AAVHSC11, AAVHSC12, AAVHSC13, AAVHSC14, AAVHSC15, AAVHSC16, AAVHSC17, and any variants thereof. In certain embodiments, the AAV capsid protein is selected from the group consisting of AAV1, AAV2, AAV5, AAV6, AAV8, AAV9, AAVrh10 and AAVrh74. In certain embodiments, the AAV capsid protein is selected from the group consisting of AAV1, AAV2, AAV5, AAV6, AAV8 and AAVrh74. The sequences of the various AAV capsid proteins are disclosed in, e.g., U.S. Patent Publication Nos.: US20030138772, US20140359799, US20150159173, US20150376607, US20170081680, and US20170360962A1, and PCT Publication No. WO2020227515, the disclosures of which are incorporated by reference herein in their entireties.

For example, in certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 203-736 of SEQ ID NO: 1, 2, 3, 4, 5, 6, 7, 8, 9, 10, 11, 12, 13, 15, 16, or 17. In certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 203-736 of SEQ ID NO: 1, 2, 3, 4, 5, 6, 7, 8, 9, 10, 11, 12, 13, 15, 16, or 17, wherein: the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G. In certain embodiments, the amino acid in the capsid protein corresponding to amino acid 626 of SEQ ID NO: 16 is G, and the amino acid in the capsid protein corresponding to amino acid 718 of SEQ ID NO: 16 is G. In certain embodiments, the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M. In certain embodiments, the amino acid in the capsid protein corresponding to amino acid 505 of SEQ ID NO: 16 is R, and the amino acid in the capsid protein corresponding to amino acid 687 of SEQ ID NO: 16 is R. In certain embodiments, the amino acid in the capsid protein corresponding to amino acid 346 of SEQ ID NO: 16 is A, and the amino acid in the capsid protein corresponding to amino acid 505 of SEQ ID NO: 16 is R. In certain embodiments, the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C. In certain embodiments, the capsid protein comprises the amino acid sequence of amino acids 203-736 of SEQ ID NO: 1, 2, 3, 4, 5, 6, 7, 8, 9, 10, 11, 12, 13, 15, 16, or 17.

For example, in certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 138-736 of SEQ ID NO: 1, 2, 3, 4, 5, 6, 7, 8, 9, 10, 11, 12, 13, 15, 16, or 17. In certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 138-736 of SEQ ID NO: 1, 2, 3, 4, 5, 6, 7, 8, 9, 10, 11, 12, 13, 15, 16, or 17, wherein: the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G. In certain embodiments, the amino acid in the capsid protein corresponding to amino acid 626 of SEQ ID NO: 16 is G, and the amino acid in the capsid protein corresponding to amino acid 718 of SEQ ID NO: 16 is G. In certain embodiments, the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M. In certain embodiments, the amino acid in the capsid protein corresponding to amino acid 505 of SEQ ID NO: 16 is R, and the amino acid in the capsid protein corresponding to amino acid 687 of SEQ ID NO: 16 is R. In certain embodiments, the amino acid in the capsid protein corresponding to amino acid 346 of SEQ ID NO: 16 is A, and the amino acid in the capsid protein corresponding to amino acid 505 of SEQ ID NO: 16 is R. In certain embodiments, the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C. In certain embodiments, the capsid protein comprises the amino acid sequence of amino acids 138-736 of SEQ ID NO: 1, 2, 3, 4, 5, 6, 7, 8, 9, 10, 11, 12, 13, 15, 16, or 17.

For example, in certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 1-736 of SEQ ID NO: 1, 2, 3, 4, 5, 6, 7, 8, 9, 10, 11, 12, 13, 15, 16, or 17. In certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 1-736 of SEQ ID NO: 1, 2, 3, 4, 5, 6, 7, 8, 9, 10, 11, 12, 13, 15, 16, or 17, wherein: the amino acid in the capsid protein corresponding to amino acid 2 of SEQ ID NO: 16 is T; the amino acid in the capsid protein corresponding to amino acid 65 of SEQ ID NO: 16 is I; the amino acid in the capsid protein corresponding to amino acid 68 of SEQ ID NO: 16 is V; the amino acid in the capsid protein corresponding to amino acid 77 of SEQ ID NO: 16 is R; the amino acid in the capsid protein corresponding to amino acid 119 of SEQ ID NO: 16 is L; the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G. In certain embodiments, the amino acid in the capsid protein corresponding to amino acid 2 of SEQ ID NO: 16 is T, and the amino acid in the capsid protein corresponding to amino acid 312 of SEQ ID NO: 16 is Q. In certain embodiments, the amino acid in the capsid protein corresponding to amino acid 65 of SEQ ID NO: 16 is I, and the amino acid in the capsid protein corresponding to amino acid 626 of SEQ ID NO: 16 is Y. In certain embodiments, the amino acid in the capsid protein corresponding to amino acid 77 of SEQ ID NO: 16 is R, and the amino acid in the capsid protein corresponding to amino acid 690 of SEQ ID NO: 16 is K. In certain embodiments, the amino acid in the capsid protein corresponding to amino acid 119 of SEQ ID NO: 16 is L, and the amino acid in the capsid protein corresponding to amino acid 468 of SEQ ID NO: 16 is S. In certain embodiments, the amino acid in the capsid protein corresponding to amino acid 626 of SEQ ID NO: 16 is G, and the amino acid in the capsid protein corresponding to amino acid 718 of SEQ ID NO: 16 is G. In certain embodiments, the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M. In certain embodiments, the amino acid in the capsid protein corresponding to amino acid 505 of SEQ ID NO: 16 is R, and the amino acid in the capsid protein corresponding to amino acid 687 of SEQ ID NO: 16 is R. In certain embodiments, the amino acid in the capsid protein corresponding to amino acid 346 of SEQ ID NO: 16 is A, and the amino acid in the capsid protein corresponding to amino acid 505 of SEQ ID NO: 16 is R. In certain embodiments, the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C. In certain embodiments, the capsid protein comprises the amino acid sequence of amino acids 1-736 of SEQ ID NO: 1, 2, 3, 4, 5, 6, 7, 8, 9, 10, 11, 12, 13, 15, 16, or 17.

In certain embodiments, the AAV capsid comprises two or more of: (a) a capsid protein comprising the amino acid sequence of amino acids 203-736 of SEQ ID NO: 1, 2, 3, 4, 6, 7, 10, 11, 12, 13, 15, 16, or 17; (b) a capsid protein comprising the amino acid sequence of amino acids 138-736 of SEQ ID NO: 1, 2, 3, 4, 5, 6, 7, 9, 10, 11, 12, 13, 15, 16, or 17; and (c) a capsid protein comprising the amino acid sequence of amino acids 1-736 of SEQ ID NO: 1, 2, 3, 4, 5, 6, 7, 8, 9, 10, 11, 12, 13, 15, 16, or 17. In certain embodiments, the AAV capsid comprises: (a) a capsid protein having an amino acid sequence consisting of amino acids 203-736 of SEQ ID NO: 1, 2, 3, 4, 6, 7, 10, 11, 12, 13, 15, 16, or 17; (b) a capsid protein having an amino acid sequence consisting of amino acids 138-736 of SEQ ID NO: 1, 2, 3, 4, 5, 6, 7, 9, 10, 11, 12, 13, 15, 16, or 17; and (c) a capsid protein having an amino acid sequence consisting of amino acids 1-736 of SEQ ID NO: 1, 2, 3, 4, 5, 6, 7, 8, 9, 10, 11, 12, 13, 15, 16, or 17.

In certain embodiments, the AAV capsid comprises one or more of: (a) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 203-736 of SEQ ID NO: 8; (b) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 138-736 of SEQ ID NO: 8; and (c) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 1-736 of SEQ ID NO: 8. In certain embodiments, the AAV capsid comprises one or more of: (a) a capsid protein comprising the amino acid sequence of amino acids 203-736 of SEQ ID NO: 8; (b) a capsid protein comprising the amino acid sequence of amino acids 138-736 of SEQ ID NO: 8; and (c) a capsid protein comprising the amino acid sequence of amino acids 1-736 of SEQ ID NO: 8. In certain embodiments, the AAV capsid comprises two or more of: (a) a capsid protein comprising the amino acid sequence of amino acids 203-736 of SEQ ID NO: 8; (b) a capsid protein comprising the amino acid sequence of amino acids 138-736 of SEQ ID NO: 8; and (c) a capsid protein comprising the amino acid sequence of amino acids 1-736 of SEQ ID NO: 8. In certain embodiments, the AAV capsid comprises: (a) a capsid protein having an amino acid sequence consisting of amino acids 203-736 of SEQ ID NO: 8; (b) a capsid protein having an amino acid sequence consisting of amino acids 138-736 of SEQ ID NO: 8; and (c) a capsid protein having an amino acid sequence consisting of amino acids 1-736 of SEQ ID NO: 8.

In certain embodiments, the AAV capsid comprises one or more of: (a) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 203-736 of SEQ ID NO: 11; (b) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 138-736 of SEQ ID NO: 11; and (c) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 1-736 of SEQ ID NO: 11. In certain embodiments, the AAV capsid comprises one or more of: (a) a capsid protein comprising the amino acid sequence of amino acids 203-736 of SEQ ID NO: 11; (b) a capsid protein comprising the amino acid sequence of amino acids 138-736 of SEQ ID NO: 11; and (c) a capsid protein comprising the amino acid sequence of amino acids 1-736 of SEQ ID NO: 11. In certain embodiments, the AAV capsid comprises two or more of: (a) a capsid protein comprising the amino acid sequence of amino acids 203-736 of SEQ ID NO: 11; (b) a capsid protein comprising the amino acid sequence of amino acids 138-736 of SEQ ID NO: 11; and (c) a capsid protein comprising the amino acid sequence of amino acids 1-736 of SEQ ID NO: 11. In certain embodiments, the AAV capsid comprises: (a) a capsid protein having an amino acid sequence consisting of amino acids 203-736 of SEQ ID NO: 11; (b) a capsid protein having an amino acid sequence consisting of amino acids 138-736 of SEQ ID NO: 11; and (c) a capsid protein having an amino acid sequence consisting of amino acids 1-736 of SEQ ID NO: 11.

In certain embodiments, the AAV capsid comprises one or more of: (a) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 203-736 of SEQ ID NO: 13; (b) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 138-736 of SEQ ID NO: 13; and (c) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 1-736 of SEQ ID NO: 13. In certain embodiments, the AAV capsid comprises one or more of: (a) a capsid protein comprising the amino acid sequence of amino acids 203-736 of SEQ ID NO: 13; (b) a capsid protein comprising the amino acid sequence of amino acids 138-736 of SEQ ID NO: 13; and (c) a capsid protein comprising the amino acid sequence of amino acids 1-736 of SEQ ID NO: 13. In certain embodiments, the AAV capsid comprises two or more of: (a) a capsid protein comprising the amino acid sequence of amino acids 203-736 of SEQ ID NO: 13; (b) a capsid protein comprising the amino acid sequence of amino acids 138-736 of SEQ ID NO: 13; and (c) a capsid protein comprising the amino acid sequence of amino acids 1-736 of SEQ ID NO: 13. In certain embodiments, the AAV capsid comprises: (a) a capsid protein having an amino acid sequence consisting of amino acids 203-736 of SEQ ID NO: 13; (b) a capsid protein having an amino acid sequence consisting of amino acids 138-736 of SEQ ID NO: 13; and (c) a capsid protein having an amino acid sequence consisting of amino acids 1-736 of SEQ ID NO: 13.

In certain embodiments, the AAV capsid comprises one or more of: (a) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the sequence of amino acids 203-736 of SEQ ID NO: 16; (b) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the sequence of amino acids 138-736 of SEQ ID NO: 16; and (c) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the sequence of amino acids 1-736 of SEQ ID NO: 16. In certain embodiments, the AAV capsid comprises one or more of: (a) a capsid protein comprising the amino acid sequence of amino acids 203-736 of SEQ ID NO: 16; (b) a capsid protein comprising the amino acid sequence of amino acids 138-736 of SEQ ID NO: 16; and (c) a capsid protein comprising the amino acid sequence of amino acids 1-736 of SEQ ID NO: 16. In certain embodiments, the AAV capsid comprises two or more of: (a) a capsid protein comprising the amino acid sequence of amino acids 203-736 of SEQ ID NO: 16; (b) a capsid protein comprising the amino acid sequence of amino acids 138-736 of SEQ ID NO: 16; and (c) a capsid protein comprising the amino acid sequence of amino acids 1-736 of SEQ ID NO: 16. In certain embodiments, the AAV capsid comprises: (a) a capsid protein having an amino acid sequence consisting of amino acids 203-736 of SEQ ID NO: 16; (b) a capsid protein having an amino acid sequence consisting of amino acids 138-736 of SEQ ID NO: 16; and (c) a capsid protein having an amino acid sequence consisting of amino acids 1-736 of SEQ ID NO: 16.

In certain embodiments, the nucleotide encoding an AAV capsid protein is operably linked to a transcriptional regulatory element that controls the expression of the AAV capsid protein. In certain embodiments, the transcriptional regulatory element comprises a promoter selected from the group consisting of a constitutive promoter, an inducible promoter, or a native promoter. Any promoter known in the art that is capable of controlling the expression of an AAV capsid protein can be used. Suitable promoters for use are known to those of skill in the art, and include, without limitation, a p40 promoter, a metallothionine (MT) promoter, a mouse mammary tumor virus (MMTV) promoter, a T7 promoter, an ecdysone insect promoter, a tetracycline-repressible promoter, a tetracycline-inducible promoter, an RU486-inducible promoter, and a rapamycin-inducible promoter. Other suitable promoters include, without limitation, a CMV promoter, a CBA promoter, and a CAG promoter.

In certain embodiments, the transcriptional regulatory element operably linked to the nucleotide sequence encoding an AAV capsid protein comprises a sequence having at least 85%, 86%, 87%, 88%, 89%, 90%, 91%, 92%, 93%, 94%, 95%, 96%, 97%, 98%, or 99% sequence identity to the sequence set forth in SEQ ID NO: 47, 48, or 49. In certain embodiments, the transcriptional regulatory element operably linked to the nucleotide sequence encoding an AAV capsid protein comprises or consists of the sequence set forth in SEQ ID NO: 47, 48, or 49.

In another aspect, the present disclosure provides a first nucleic acid vector comprising a first nucleotide sequence comprising a Rep-Cap element, and a second nucleotide sequence comprising an rAAV genome comprising a transgene. In certain embodiments, the Rep-Cap element comprises a nucleic acid sequence encoding an AAV Rep protein and a nucleic acid sequence encoding an AAV capsid protein. The Rep-Cap element can comprise a nucleic acid sequence encoding any AAV Rep protein known in the art and a nucleic acid sequence encoding any AAV capsid protein known in the art. In certain embodiments, the Rep-Cap element comprises a nucleotide sequence having at least 85%, 86%, 87%, 88%, 89%, 90%, 91%, 92%, 93%, 94%, 95%, 96%, 97%, 98%, or 99% sequence identity to the sequence set forth in SEQ ID NO: 73 or 77.

### III. Second Nucleic Acid Vector

The dual vector transfection system described herein generally comprises a second nucleic acid vector comprising one or more helper virus genes. As is appreciated by those of skill in the art, the replication of AAV depends on the presence of helper factors that are encoded by helper virus genes. Helper factors can be provided via coinfections by helper viruses, such as a helper virus from, without limitation, adenovirus, herpesvirus, papillomavirus, cytomegalovirus, baculovirus and human bocavirus. However, growing AAV in the presence of a helper virus can lead to the lysis of host cells and/or contamination of the AAV product. As such, the genes of the helper virus that encode helper factors required for AAV replication can be provided on a vector that is used to transfect host cells.

The dual vector transfection system described herein generally involves the transfection of two nucleic acid vectors into a host cell for AAV (e.g., rAAV) production: (1) a first nucleic acid vector comprising a first nucleotide sequence encoding an AAV Rep protein, a second nucleotide sequence comprising an rAAV genome comprising a transgene, and a third nucleotide sequence encoding an AAV capsid protein; and (2) a second nucleic acid vector comprising a helper virus gene. In certain embodiments, the second nucleic acid vector does not comprise any component of AAV production that is found in the first nucleic acid vector. In certain embodiments, the second nucleic acid vector does not comprise an rAAV genome comprising a transgene. In certain embodiments, the second nucleic acid vector does not comprise an AAV capsid protein coding sequence. In certain embodiments, the second nucleic acid vector does not comprise a Rep coding sequence or a coding sequence of a functional fragment thereof. In certain embodiments, the second nucleic acid vector does not comprise an rAAV genome comprising a transgene, the second nucleic acid vector does not comprise an AAV capsid protein coding sequence, and/or the second nucleic acid vector does not comprise a Rep coding sequence or a coding sequence of a functional fragment thereof.

In certain embodiments, the second nucleic acid vector comprises at least one helper virus gene that may be derived from a helper virus selected from the group consisting of adenovirus, herpesvirus, poxvirus, cytomegalovirus, and baculovirus. The helper virus gene may be operably linked to a transcriptional regulatory element that controls the expression of the helper virus gene. In certain embodiments, the transcriptional regulatory element comprises a promoter selected from the group consisting of a constitutive promoter, an inducible promoter, or a native promoter. Suitable promoters for use are known to those of skill in the art, and include, without limitation, an RSV LTR promoter, a CMV immediate early promoter, an SV40 promoter, a dihydrofolate reductase promoter, a cytoplasmic β-actin promoter, a phosphoglycerate kinase (PGK) promoter, a metallothionine (MT) promoter, a mouse mammary tumor virus (MMTV) promoter, a T7 promoter, an ecdysone insect promoter, a tetracycline-repressible promoter, a tetracycline-inducible promoter, an RU486-inducible promoter, and a rapamycin-inducible promoter.

In certain embodiments, the second nucleic acid vector comprises at least one helper virus gene. The at least one helper virus gene may be derived from adenovirus (AdV). The minimal set of AdV helper factors that are known to be required for efficient AAV production consists of the AdV molecules E1, E2, E4, and VA RNA (see, e.g., Meier et al. (2020) Viruses 12(6): 662). In particular, the minimal set of AdV helper factors required for efficient AAV production includes the AdV molecules E1A, E1B, E2A, E4, and VA RNA. In certain embodiments, the second nucleic acid vector comprises a sufficient set of helper virus genes that will allow for efficient AAV production (e.g., AAV replication and packaging) in the host cell (e.g., host AAV production cell).

The typical AdV genome expresses about 40 tightly regulated proteins that are divided into an early and a late phase. Early phase proteins include E1A, E1B, E2A, and E4. Briefly, E1A and E2A proteins function to activate the AAV promoters p5 and p19 that control the expression of AAV Rep proteins. E1A mediated p5 activity has been found to be required for AAV replication. E2A is a single-stranded DNA binding protein that has been shown to facilitate various aspects of AAV replication. The E1B gene encodes for E1B19K and E1B55K oncoproteins. E1B19K inhibits E1A induced apoptosis, and E1B55K inhibits the tumor suppressor protein p53. E1B55K functions together with E4orf6 to promote AAV second-strand synthesis and viral DNA replication. E1B55K has also been shown to facilitate AAV mRNA export and inhibit cellular mRNA export, together facilitating AAV gene expression. E1B19K has been found to function in enhancing AAV titers when co-expressed with other AdV helper factors such as E1A, E1B55K, E2A, and E4orf6.

The VA RNA has been found to function in inhibiting the cellular innate immune protein double-stranded RNA-activated kinase (PKR), the inhibition of which ensures efficient virus protein synthesis. VA RNA has also been shown to facilitate the synthesis and assembly of AAV structural proteins. It will be readily appreciated to those of skill in the art that the VA nucleic acid within the AdV genome is a non-translated nucleic acid sequence that gives rise to the VA RNA.

One of the most commonly used helper functions comes from the human AdV type 5. Adenoviral helper virus genes may also be derived from other known adenoviruses, for example, AdV type 2. The AdV5 genome is about 36 kilobases and an exemplary AdV5 genome sequence can be found via NCBI Reference Sequence AC 000008.1. According to the NCBI Reference Sequence, E1A is encoded by nucleotides 560 to 1545; E1B19K is encoded by nucleotides 1714 to 2244; E1B55K is encoded by nucleotides 2019 to 3509; E2A is encoded by nucleotides 22443 to 24032; and E4orf6/7 is encoded by nucleotides 32914 to 34077.

In certain embodiments, the present disclosure provides a nucleic acid comprising a nucleotide sequence encoding AdV5 E2A. In certain embodiments, the nucleic acid comprising a nucleotide sequence encoding AdV5 E2A comprises a transcriptional regulatory element operably linked to the nucleotide sequence encoding AdV5 E2A. In certain embodiments, the nucleic acid comprising a nucleotide sequence encoding AdV5 E2A comprises a sequence having at least 85%, 86%, 87%, 88%, 89%, 90%, 91%, 92%, 93%, 94%, 95%, 96%, 97%, 98%, or 99% sequence identity to the sequence set forth in SEQ ID NO: 60. In certain embodiments, the nucleic acid comprising a nucleotide sequence encoding AdV5 E2A comprises or consists of the sequence set forth in SEQ ID NO: 60. In certain embodiments, the present disclosure provides a nucleic acid comprising a nucleotide sequence corresponding to the sequence encoding E2A as described for AdV5, in a different adenovirus serotype (e.g., AdV2).

In certain embodiments, the present disclosure provides a nucleic acid comprising a nucleotide sequence encoding AdV5 E4. In certain embodiments, the nucleic acid comprising a nucleotide sequence encoding AdV5 E4 comprises a transcriptional regulatory element operably linked to the nucleotide sequence encoding AdV5 E4. In certain embodiments, the nucleic acid comprising a nucleotide sequence encoding AdV5 E4 comprises a sequence having at least 85%, 86%, 87%, 88%, 89%, 90%, 91%, 92%, 93%, 94%, 95%, 96%, 97%, 98%, or 99% sequence identity to the sequence set forth in SEQ ID NO: 61. In certain embodiments, the nucleic acid comprising a nucleotide sequence encoding AdV5 E4 comprises or consists of the sequence set forth in SEQ ID NO: 61. In certain embodiments, the present disclosure provides a nucleic acid comprising a nucleotide sequence corresponding to the sequence encoding E4 as described for AdV5, in a different adenovirus serotype (e.g., AdV2).

In certain embodiments, the present disclosure provides a nucleic acid comprising a nucleotide sequence encoding AdV5 VA RNA. In certain embodiments, the nucleic acid comprising a nucleotide sequence encoding AdV5 VA RNA comprises a transcriptional regulatory element operably linked to the nucleotide sequence encoding AdV5 VA RNA. In certain embodiments, the nucleic acid comprising a nucleotide sequence encoding AdV5 VA RNA comprises a sequence having at least 85%, 86%, 87%, 88%, 89%, 90%, 91%, 92%, 93%, 94%, 95%, 96%, 97%, 98%, or 99% sequence identity to the sequence set forth in SEQ ID NO: 62. In certain embodiments, the nucleic acid comprising a nucleotide sequence encoding AdV5 VA RNA comprises or consists of the sequence set forth in SEQ ID NO: 62. It will be readily appreciated to those of skill in the art that the VA RNA nucleic acid sequence is a non-translated nucleic acid sequence that gives rise to (e.g., “encodes”) the VA RNA. In certain embodiments, the present disclosure provides a nucleic acid comprising a nucleotide sequence corresponding to the sequence encoding VA RNA as described for AdV5, in a different adenovirus serotype (e.g., AdV2).

In certain embodiments, the present disclosure provides a nucleic acid comprising a nucleotide sequence encoding AdV5 E2A, E4, and VA RNA. In certain embodiments, the nucleic acid comprising a nucleotide sequence encoding AdV5 E2A, E4, and VA RNA comprises one or more transcriptional regulatory elements that may be operably linked to each of the nucleotide sequences encoding AdV5 E2A, E4, and VA RNA. In certain embodiments, the nucleic acid comprising a nucleotide sequence encoding AdV5 E2A, E4, and VA RNA comprises a sequence having at least 85%, 86%, 87%, 88%, 89%, 90%, 91%, 92%, 93%, 94%, 95%, 96%, 97%, 98%, or 99% sequence identity to the sequence set forth in SEQ ID NO: 63. In certain embodiments, the nucleic acid comprising a nucleotide sequence encoding AdV5 E2A, E4, and VA RNA comprises or consists of the sequence set forth in SEQ ID NO: 63.

In certain embodiments, the present disclosure provides a nucleic acid comprising a nucleotide sequence encoding the minimal set of AdV helper factors required for efficient AAV production. In certain embodiments, the nucleic acid comprising a nucleotide encoding the minimal set of AdV helper factors encode the AdV molecules E1A, E1B, E2A, E4, and VA RNA.

Certain host cells such as HEK293T cells endogenously provide some, but not all required helper factors, and the remaining helper factors can be provided exogenously via plasmid transfection. For example, HEK293T cells endogenously express adenoviral E1A and E1B genes, and are provided with the remaining adenoviral helper genes, i.e., those that encode AdV5 E4, E2A, and virus-associated (VA) RNA. Such AdV5 helper genes may be provided by a single vector through transfection. In certain embodiments, the present disclosure provides a second nucleic acid vector comprising an AdV5 helper virus gene selected from the group consisting of E2A, E4, and VA RNA. In certain embodiments, the present disclosure provides a second nucleic acid vector comprising a helper virus gene that encodes for E2A, E4, and VA RNA as described for AdV5, derived from a different adenovirus serotype (e.g., AdV2).

Helper virus genes may also be derived from herpesviruses, papillomaviruses, and human bocavirus. Examples of herpesvirus from which a helper virus factor can be derived include HSV-1 and HSV-2. Helper virus factors derived from HSV-1 that are known to be involved in supporting AAV production include, without limitation, UL5, UL8, UL52, ICP8, ICP0, ICP4, ICP22, UL30, and UL42. The various functions of these HSV-1 helper virus factors and how they support AAV production are known to those of skill in the art. For example, the HSV-1 helicase-primase complex UL5/UL8/UL52 in addition to the single-strand DNA binding protein ICP8 is known to be sufficient in the restoring of AAV progeny production in an AAV infection model; ICP0, ICP4, and ICP22 are implicated to promote expression of Rep protein; and the HSV-1 DNA polymerase UL30/UL42 is implicated in the replication of AAV DNA. Accordingly, in certain embodiments the second nucleic acid vector comprises at least one helper virus gene selected from the group consisting of UL5, UL8, UL52, ICP8, ICP0, ICP4, ICP22, UL30, and UL42. An example of papillomavirus from which a helper virus factor can be derived is HPV-16. In certain embodiments, helper virus factors derived from HPV-16 can enhance AAV production in the presence of AdV helper factors. Such HPV-16 helper factors that are known to be involved in supporting AAV replication include, without limitation, E1, E2, and E6. An example of human bocavirus from which a helper virus factor can be derived is human bocavirus 1 (HBoV1). Helper virus factors derived from HBoV1 that are known to be involved in supporting AAV production include, without limitation, NP1, NS2, NS4, and the viral long noncoding RNA BocaSR.

### IV. Vectors and Cells

The present disclosure provides a first nucleic acid vector comprising a first nucleotide sequence encoding an AAV Rep protein, a second nucleotide sequence comprising an rAAV genome comprising a transgene, and a third nucleotide sequence encoding an AAV capsid protein; and a second nucleic acid vector comprising a helper virus gene.

The first nucleic acid vector and the second nucleic acid vector can independently be any form of nucleic acid vector. Suitable vectors, include, without limitation, plasmids, minimal vectors (e.g., minicircles, Nanoplasmids™, doggybones, MIDGE vectors, and the like), viruses, cosmids, artificial chromosomes, linear DNA, and mRNA. In certain embodiments, the first nucleic acid vector and/or the second nucleic acid vector is a DNA plasmid or a DNA minimal vector. Any DNA plasmid or DNA minimal vector that can accommodate the necessary vector elements can be used for the first nucleic acid vector and the second nucleic acid vector. Suitable DNA minimal vectors include, without limitation, linear covalently closed DNA (e.g., ministring DNA), linear covalently closed dumbbell shaped DNA (e.g., doggybone DNA, dumbbell DNA), minicircles, Nanoplasmids™, minimalistic immunologically defined gene expression (MIDGE) vectors, and others known to those of skill in the art. DNA minimal vectors and their methods of production are described in, e.g., U.S. Patent Application Nos. 20100233814, 20120282283, 20130216562, 20150218565, 20150218586, 20160008488, 20160215296, 20160355827, 20190185924, 20200277624, and 20210010021, all of which are herein incorporated by reference in their entireties.

In certain embodiments, the nucleic acids in the vectors disclosed herein are optimized, e.g., by codon/RNA optimization, replacement with heterologous signal sequences, and/or elimination of mRNA instability elements. Methods to generate optimized polynucleotides for recombinant expression by introducing codon changes and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly, all of which are herein incorporated by reference in their entireties. For example, potential splice sites and instability elements (e.g., A/T or A/U rich elements) within the RNA can be mutated without altering the amino acids encoded by the nucleic acid sequences to increase stability of the RNA for recombinant expression. The alterations utilize the degeneracy of the genetic code, e.g., using an alternative codon for an identical amino acid. In certain embodiments, it can be desirable to alter one or more codons to encode a conservative mutation, e.g., a similar amino acid with similar chemical structure and properties and/or function as the original amino acid. Such methods can increase expression of the encoded capsid protein relative to the expression of the capsid encoded by polynucleotides that have not been optimized.

The vectors disclosed herein can be introduced into cells (using any techniques known in the art) for propagation of the vectors and/or for expression of a protein encoded by the vector. Accordingly, in another aspect, the present disclosure provides a recombinant cell comprising a vector disclosed herein. And further, in another aspect, the present disclosure provides a method of producing an rAAV, the method comprising culturing the recombinant cell under conditions whereby the polynucleotide is expressed and the rAAV is produced.

A variety of host cells and expression systems can be utilized. Such expression systems represent vehicles by which the coding sequences of interest can be produced and subsequently purified, but also represent cells which can, when transformed or transfected with the appropriate nucleotide coding sequences described herein, produce rAAV. These include but are not limited to microorganisms such as bacteria (e.g., E. coli and B. subtilis) transformed with, e.g., recombinant bacteriophage DNA, plasmid DNA, or cosmid DNA expression vectors containing the nucleotide coding sequences described herein; yeast (e.g., Saccharomyces Pichia) transformed with, e.g., recombinant yeast expression vectors containing the nucleotide coding sequences described herein; insect cell systems infected with, e.g., recombinant virus expression vectors (e.g., baculovirus) containing the nucleotide coding sequences described herein; plant cell systems (e.g., green algae such as Chlamydomonas reinhardtii) infected with, e.g., recombinant virus expression vectors (e.g., cauliflower mosaic virus, CaMV; tobacco mosaic virus, TMV) or transformed with, e.g., recombinant plasmid expression vectors (e.g., Ti plasmid) containing the nucleotide coding sequences described herein; or mammalian cell systems (e.g., COS (e.g., COS1 or COS), CHO, BHK, MDCK, HEK293, NSO, PER.C6, VERO, CRL7O3O, HsS78Bst, HeLa, and NIH 3T3, HEK293T, HEK293F, HepG2, SP210, R1.1, B-W, L-M, BSC1, BSC40, YB/20 and BMT10 cells) harboring, e.g., recombinant expression constructs containing the nucleotide coding sequences described herein comprising promoters derived from the genome of mammalian cells (e.g., metallothionein promoter) or from mammalian viruses (e.g., the adenovirus late promoter; the vaccinia virus 7.5K promoter). In certain embodiments, cells for expressing the nucleotide coding sequences described herein are human cells, e.g., human cell lines. In certain embodiments, a mammalian expression vector is pOptiVEC™ or pcDNA3.3. In certain embodiments, bacterial cells such as Escherichia coli, or eukaryotic cells (e.g., mammalian cells), are used for the expression of the nucleotide coding sequences described herein. For example, mammalian cells such as CHO or HEK293 cells, in conjunction with a vector element such as the major intermediate early gene promoter element from human cytomegalovirus is an effective expression system for the polynucleotides described herein.

In bacterial systems, a number of expression vectors can be advantageously selected depending upon the use intended for the protein being expressed. For example, when a large quantity of a protein is to be produced, vectors which direct the expression of high levels of fusion protein products that are readily purified can be desirable. Such vectors include, but are not limited to, the E. coli expression vector pUR278 (Ruether U & Mueller-Hill B (1983) EMBO J 2: 1791-1794), in which the protein coding sequence can be ligated individually into the vector in frame with the lac Z coding region so that a fusion protein is produced; pIN vectors (Inouye S & Inouye M (1985) Nuc Acids Res 13: 3101-3109; Van Heeke G & Schuster S M (1989) J Biol Chem 24: 5503-5509); and the like, all of which are herein incorporated by reference in their entireties. For example, pGEX vectors can also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV), for example, can be used as a vector to express foreign genes. The virus grows in Spodoptera frugiperda cells. The protein coding sequence can be cloned individually into non-essential regions (for example the polyhedrin gene) of the virus and placed under control of an AcNPV promoter (for example the polyhedrin promoter).

In mammalian host cells, a number of viral-based expression systems can be utilized. In cases where an adenovirus is used as an expression vector, the protein coding sequence of interest can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing the nucleotide coding sequences described herein in infected hosts (See, e.g., Logan J & Shenk T (1984) PNAS 81(12): 3655-9, which is herein incorporated by reference in its entirety). Specific initiation signals can also be required for efficient translation of inserted protein coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (see, e.g., Bitter G et al. (1987) Methods Enzymol. 153: 516-544, which is herein incorporated by reference in its entirety).

In addition, a host cell strain can be chosen which modulates the expression of the inserted sequences, or modifies and processes the gene product in the specific fashion desired. Such modifications (e.g., glycosylation) and processing (e.g., cleavage) of protein products can be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product can be used. Such mammalian host cells include but are not limited to CHO, VERO, BHK, Hela, MDCK, HEK293, HEK293T, HEK293F, HEK293EBNA, NIH 3T3, W138, BT483, Hs578T, HTB2, BT2O and T47D, NSO (a murine myeloma cell line that does not endogenously produce any immunoglobulin chains), CAP, CAP-T, CRL7O3O, COS (e.g., COS1 or COS), PER.C6, VERO, AGE1.CR, A549, HsS78Bst, HepG2, C139, EB66, SP210, R1.1, B-W, L-M, BSC1, BSC40, YB/20, BMT10 and HsS78Bst cells.

In certain embodiments, rather than using expression vectors which contain viral origins of replication, host cells can be transformed with a polynucleotide (e.g., DNA or RNA) controlled by appropriate transcriptional regulatory elements (e.g., promoter, enhancer, sequences, transcription terminators, polyadenylation sites, etc.), and a selectable marker. Following the introduction of polynucleotide, engineered cells can be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines which express a protein described herein or a fragment thereof.

A number of selection systems can be used, including but not limited to the herpes simplex virus thymidine kinase (Wigler M et al. (1977) Cell 11(1): 223-32), hypoxanthineguanine phosphoribosyltransferase (Szybalska E H & Szybalski W (1962) PNAS 48(12): 2026-2034), and adenine phosphoribosyltransferase (Lowy I et al. (1980) Cell 22(3): 817-23) genes in tk-, hgprt- or aprt-cells, respectively, all of which are herein incorporated by reference in their entireties. Also, antimetabolite resistance can be used as the basis of selection for the following genes: dhfr, which confers resistance to methotrexate (Wigler M et al. (1980) PNAS 77(6): 3567-70; O'Hare K et al. (1981) PNAS 78: 1527-31); gpt, which confers resistance to mycophenolic acid (Mulligan R C & Berg P (1981) PNAS 78(4): 2072-6); neo, which confers resistance to the aminoglycoside G-418 (Wu G Y & Wu C H (1991) Biotherapy 3: 87-95; Tolstoshev P (1993) Ann Rev Pharmacol Toxicol 32: 573-596; Mulligan R C (1993) Science 260: 926-932; and Morgan R A & Anderson W F (1993) Ann Rev Biochem 62: 191-217; Nabel G J & Felgner P L (1993) Trends Biotechnol 11(5): 211-5); and hygro, which confers resistance to hygromycin (Santerre R F et al. (1984) Gene 30(1-3): 147-56), all of which are herein incorporated by reference in their entireties. Methods commonly known in the art of recombinant DNA technology can be routinely applied to select the desired recombinant clone and such methods are described, for example, in Ausubel F M et al. (eds.), Current Protocols in Molecular Biology, John Wiley & Sons, N Y (1993); Kriegler M, Gene Transfer and Expression, A Laboratory Manual, Stockton Press, N Y (1990); and in Chapters 12 and 13, Dracopoli N C et al. (eds.), Current Protocols in Human Genetics, John Wiley & Sons, N Y (1994); Colbère-Garapin F et al. (1981) J Mol Biol 150: 1-14, all of which are herein incorporated by reference in their entireties.

### V. Adeno-Associated Virus Packaging Systems and Methods

In another aspect, the present disclosure provides packaging systems for recombinant preparation of a recombinant adeno-associated virus (rAAV) disclosed herein. In particular, the present disclosure provides packaging systems useful for AAV production under a dual vector transfection system described herein (e.g., AAV production is mediated by the use of a packaging system that comprises a first and a second nucleic acid vector delivered into a host cell). Such packaging systems generally comprise or consist of: (1) a first nucleic acid vector comprising a first nucleotide sequence encoding an AAV Rep protein, a second nucleotide sequence comprising an rAAV genome comprising a transgene, and a third nucleotide sequence encoding an AAV capsid protein; and (2) a second nucleic acid vector comprising a helper virus gene. The first nucleic acid vector and the second nucleic acid vector together are capable of providing all the components needed for the production of rAAV. In certain embodiments, components required for the production of rAAV are provided by the host cell from which rAAV are produced. In such an embodiment, the first nucleic acid vector and the second nucleic acid vector together with the host cell, are capable of providing all the components needed for the production of rAAV. The packaging systems described herein are operative in a cell for enclosing the rAAV genome in a capsid to form the rAAV.

In certain embodiments, the present disclosure provides an rAAV packaging system comprising: (1) a first nucleic acid vector comprising a first nucleotide sequence encoding an AAV Rep protein, a second nucleotide sequence comprising an rAAV genome comprising a transgene, and a third nucleotide sequence encoding an AAV capsid protein; and (2) a second nucleic acid vector comprising a helper virus gene. In certain embodiments, the present disclosure provides an rAAV packaging system comprising: (1) a first nucleic acid vector comprising from 5′ to 3′, a first nucleotide sequence encoding an AAV Rep protein, a second nucleotide sequence comprising an rAAV genome comprising a transgene, and a third nucleotide sequence encoding an AAV capsid protein; and (2) a second nucleic acid vector comprising a helper virus gene.

In certain embodiments, the first nucleic acid vector of the packaging system comprises an rAAV genome comprising a transgene. The first nucleic acid vector of the packaging system of the present disclosure further comprises an AAV Rep protein coding sequence or a coding sequence of a functional variant thereof, and an AAV capsid protein coding sequence. Accordingly, the present disclosure provides a first nucleic acid vector of the packaging system comprising a first nucleotide sequence encoding an AAV Rep protein or a functional variant thereof, a second nucleotide sequence comprising an rAAV genome comprising a transgene, and a third nucleotide sequence encoding an AAV capsid protein. In certain embodiments, the first nucleic acid vector of the packaging system comprises from 5′ to 3′: a first nucleotide sequence encoding an AAV Rep protein or a functional variant thereof, a second nucleotide sequence comprising an rAAV genome comprising a transgene, and a third nucleotide sequence encoding an AAV capsid protein. In certain embodiments, the first nucleic acid vector of the packaging system does not comprise a helper virus gene.

Any AAV Rep protein can be employed in the packaging systems disclosed herein. In certain embodiments of the packaging system, the Rep nucleotide sequence encodes an AAV2 Rep protein. Suitable AAV2 Rep proteins may include, without limitation, Rep 78/68 or Rep 68/52. In certain embodiments of the packaging system, the nucleotide sequence encoding the AAV2 Rep protein comprises a nucleotide sequence that encodes a protein having a minimum percent sequence identity to the AAV2 Rep amino acid sequence of SEQ ID NO: 64, wherein the minimum percent sequence identity is at least 70% (e.g., at least 75%, at least 80%, at least 85%, at least 90%, at least 95%, at least 98%, at least 99%, or 100%) across the length of the amino acid sequence of the AAV2 Rep protein. In certain embodiments of the packaging system, the AAV2 Rep protein has the amino acid sequence set forth in SEQ ID NO: 64.

In certain embodiments, the second nucleic acid vector of the packaging system comprises a helper virus gene. The second nucleic acid vector of the packaging system of the present disclosure may comprise one or more helper virus genes. Certain aspects of the present disclosure provide that the second nucleic acid vector of the packaging system does not comprise any component of AAV production that is found in a first nucleic acid vector as described herein. In certain embodiments, the second nucleic acid vector of the packaging system does not comprise an rAAV genome comprising a transgene. In certain embodiments, the second nucleic acid vector of the packaging system does not comprise an AAV capsid protein coding sequence. In certain embodiments, the second nucleic acid vector of the packaging system does not comprise a Rep coding sequence or a coding sequence of a functional variant thereof. In certain embodiments, the second nucleic acid vector of the packaging system does not comprise an rAAV genome comprising a transgene, the second nucleic acid vector of the packaging system does not comprise an AAV capsid protein coding sequence, and/or the second nucleic acid vector of the packaging system does not comprise a Rep coding sequence or a coding sequence of a functional variant thereof.

In certain embodiments of the packaging system, the helper virus is selected from the group consisting of adenovirus, herpes virus (including herpes simplex virus (HSV)), poxvirus (such as vaccinia virus), cytomegalovirus (CMV), and baculovirus. In certain embodiments of the packaging system, where the helper virus is adenovirus, the adenovirus genome comprises one or more adenovirus RNA genes selected from the group consisting of E1, E2, E4, and VA. In certain embodiments of the packaging system, where the adenovirus genome comprises one or more adenovirus RNA genes selected from the group consisting of E2, E4, and VA. In certain embodiments of the packaging system, where the helper virus is HSV, the HSV genome comprises one or more of HSV genes selected from the group consisting of UL5/8/52, ICPO, ICP4, ICP22, and UL30/UL42.

In certain embodiments of the packaging system, the first and second nucleic acid vector of the packaging system are contained within two plasmids. In certain embodiments, the first nucleic acid vector of the packaging system is contained within a first plasmid. In certain embodiments, the second nucleic acid vector of the packaging system is contained within a second plasmid.

In certain embodiments of the packaging system, the first and second nucleic acid vector of the packaging system are contained within two recombinant helper viruses. In certain embodiments, the first nucleic acid vector of the packaging system is contained within a first recombinant helper virus. In certain embodiments, the second nucleic acid vector of the packaging system is contained within a second recombinant helper virus. In certain embodiments, the first and second nucleic acid vector of the packaging system are contained within a single recombinant helper virus.

In a further aspect, the present disclosure provides a method for recombinant preparation of an rAAV, wherein the method comprises transfecting or transducing a cell with a packaging system as described herein under conditions operative for enclosing the rAAV genome in the capsid to form the rAAV. Exemplary methods for recombinant preparation of an rAAV include transient transfection (e.g., with one or more transfection plasmids), viral infection (e.g., with one or more recombinant helper viruses, such as an adenovirus, poxvirus (such as vaccinia virus), herpes virus (including HSV, cytomegalovirus, or baculovirus)), and stable producer cell line transfection or infection (e.g., with a stable producer cell, such as a mammalian or insect cell).

Accordingly, the present disclosure provides a packaging system for preparation of an rAAV, wherein the packaging system comprises: (1) a first nucleic acid vector comprising: a first nucleotide sequence encoding an AAV Rep protein or a functional variant thereof; a second nucleotide sequence comprising an rAAV genome; and a third nucleotide sequence encoding an AAV capsid protein, and (2) a second nucleic acid vector comprising a helper virus gene. In certain embodiments, the present disclosure provides a packaging system for preparation of an rAAV, wherein the packaging system comprises: (1) a first nucleic acid vector comprising from 5′ to 3′: a first nucleotide sequence encoding an AAV Rep protein or a functional variant thereof; a second nucleotide sequence comprising an rAAV genome; and a third nucleotide sequence encoding an AAV capsid protein, and (2) a second nucleic acid vector comprising a helper virus gene.

Accordingly, the present disclosure provides a method for recombinant preparation of an rAAV, wherein the method comprises transfecting or transducing a cell with a packaging system comprising: (1) a first nucleic acid vector comprising: a first nucleotide sequence encoding an AAV Rep protein or a functional variant thereof; a second nucleotide sequence comprising an rAAV genome; and a third nucleotide sequence encoding an AAV capsid protein, and (2) a second nucleic acid vector comprising a helper virus gene. In certain embodiments, the present disclosure provides a method for recombinant preparation of an rAAV, wherein the method comprises transfecting or transducing a cell with a packaging system comprising: (1) a first nucleic acid vector comprising from 5′ to 3′: a first nucleotide sequence encoding an AAV Rep protein or a functional variant thereof; a second nucleotide sequence comprising an rAAV genome; and a third nucleotide sequence encoding an AAV capsid protein, and (2) a second nucleic acid vector comprising a helper virus gene.

In certain embodiments, the total amount of nucleic acid that is transfected or transduced into the cell, including (1) a first nucleic acid vector comprising: a first nucleotide sequence encoding an AAV Rep protein or a functional variant thereof; a second nucleotide sequence comprising an rAAV genome; and a third nucleotide sequence encoding an AAV capsid protein, and (2) a second nucleic acid vector comprising a helper virus gene, is from 0.1 DNA/1E6 cells to 4 μg DNA/1E6 cells. For example, the total amount of nucleic acid that is transfected or transduced into the cell, including the first nucleic acid vector and the second nucleic acid vector, is 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, or 4 μg DNA/1E6 cells. In certain embodiments, the total amount of nucleic acid that is transfected or transduced into the cell, including the first nucleic acid vector and the second nucleic acid vector, is 1 μg DNA/1E6 cells. In certain embodiments, the total amount of nucleic acid that is transfected or transduced into the cell, including the first nucleic acid vector and the second nucleic acid vector, is 0.6 μg DNA/1E6 cells. In certain embodiments, the total amount of nucleic acid that is transfected or transduced into the cell, including the first nucleic acid vector and the second nucleic acid vector, is 0.7 μg DNA/1E6 cells. In certain embodiments, the total amount of nucleic acid that is transfected or transduced into the cell, including the first nucleic acid vector and the second nucleic acid vector, is 0.75 μg DNA/1E6 cells. In certain embodiments, the total amount of nucleic acid that is transfected or transduced into the cell, including the first nucleic acid vector and the second nucleic acid vector, is 0.8 μg DNA/1E6 cells. In certain embodiments, the total amount of nucleic acid that is transfected or transduced into the cell, including the first nucleic acid vector and the second nucleic acid vector, is 0.9 μg DNA/1E6 cells.

In certain embodiments, the ratio of the first nucleic acid vector to the second nucleic acid vector or the ratio of the second nucleic acid vector to the first nucleic acid vector is from 1:0.1 to 1:20. For example, the ratio of the first nucleic acid vector to the second nucleic acid vector or the ratio of the second nucleic acid vector to the first nucleic acid vector is 1:0.1, 1:0.2, 1:0.3, 1:0.4, 1:0.5, 1:0.6, 1:0.7, 1:0.8, 1:1, 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, 1:3, 1:3.1, 1:3.2, 1:3.2, 1:3.3, 1:3.4, 1:3.5, 1:3.6, 1:3.7, 1:3.8, 1:3.9, 1:4, 1.4.5, 1:5, 1:5.5, 1:6, 1:6.5, 1:7, 1:7.5, 1:8, 1:8.5, 1:9, 1:9.5, 1:10, 1:10.5, 1:11, 1:11.5, 1:12, 1:12.5, 1:13, 1:13.5, 1:14, 1:14.5, 1:15, 1:15.5, 1:16, 1:16.5, 1:17, 1:17.5, 1:18, 1:18.5, 1:19, 1:19.5, or 1:20. In certain embodiments, the ratio of the first nucleic acid vector to the second nucleic acid vector or the ratio of the second nucleic acid vector to the first nucleic acid vector is selected from the group consisting of: 1:0.2, 1:0.4, 1:0.6, 1:0.8, 1:1, 1:2, 1:3, or 1:4. In certain embodiments, the ratio of the first nucleic acid vector to the second nucleic acid vector or the ratio of the second nucleic acid vector to the first nucleic acid vector is 1:2. In certain embodiments, the ratio of the first nucleic acid vector to the second nucleic acid vector or the ratio of the second nucleic acid vector to the first nucleic acid vector is from 1:0.2 to 1:1. In certain embodiments, the ratio of the first nucleic acid vector to the second nucleic acid vector or the ratio of the second nucleic acid vector to the first nucleic acid vector is 1:0.6. In certain embodiments, the ratio of the first nucleic acid vector to the second nucleic acid vector or the ratio of the second nucleic acid vector to the first nucleic acid vector is 1:0.8. In certain embodiments, the ratio of the first nucleic acid vector to the second nucleic acid vector or the ratio of the second nucleic acid vector to the first nucleic acid vector is 1:1.

In certain embodiments, a method for recombinant preparation of an rAAV disclosed herein results in an increased rAAV titer as compared to a method that comprises producing rAAV using a mammalian cell comprising: (i) a first vector comprising a nucleotide sequence encoding the AAV Rep protein and the AAV capsid protein; (ii) a second vector comprising the rAAV genome; and (iii) a third vector comprising the one or more helper virus genes. In certain embodiments, a method for recombinant preparation of an rAAV disclosed herein results in an increased rAAV titer as compared to a method that comprises producing rAAV using a mammalian cell comprising: (i) a first vector comprising a nucleotide sequence encoding the AAV Rep protein and the AAV capsid protein; (ii) a second vector comprising the rAAV genome; and (iii) a third vector comprising the one or more helper virus genes.

In certain embodiments, the mammalian cell is provided in a cell culture. In certain embodiments, the cell culture has a volume of at least 2 liters, at least 50 liters, or at least 2000 liters. In certain embodiments, the cell culture has a volume of about 2 liters to about 5000 liters. In certain embodiments, the cell culture has a volume of about 2 liters to about 4000 liters. In certain embodiments, the cell culture has a volume of about 2 liters to about 3000 liters. In certain embodiments, the cell culture has a volume of about 2 liters to about 2500 liters. In certain embodiments, the cell culture has a volume of about 2 liters to about 2000 liters. In certain embodiments, the cell culture has a volume of about 2 liters to about 1500 liters. In certain embodiments, the cell culture has a volume of about 2 liters to about 1000 liters. In certain embodiments, the cell culture has a volume of about 2 liters to about 500 liters. In certain embodiments, the cell culture has a volume of about 2 liters to about 250 liters. In certain embodiments, the cell culture has a volume of about 2 liters to about 100 liters. In certain embodiments, the cell culture has a volume of about 2 liters to about 50 liters. In certain embodiments, the cell culture has a volume of about 2 liters to about 25 liters. In certain embodiments, the methods described herein are carried out in a bioreactor having a volume of at least 2 liters, at least 50 liters, or at least 2000 liters. In certain embodiments, the methods described herein are carried out in a bioreactor having a volume of 2000 liters.

### EXAMPLES

The following examples are offered by way of illustration, and not by way of limitation.

### Example 1: Materials and Methods

The following general materials and methods were used in the following Examples.

Small scale production: HEK293 cells were expanded for at least one passage and inoculated into a shake flask containing the appropriate amount of cell culture medium prior to transfection. Shake flasks were incubated in a shaker at 37° C., 8% CO₂, and 135 rpm. Cells were transfected when the cells reached a density of 1.8E6 to 2.4E6 cells/mL (for Examples 1-8) or 3.6E6 to 5E6 cells/mL (for Example 9). Transfection mixes were prepared by mixing calculated volumes of vector(s), OptiPro media, and polyethylenimine (PEI), all at ambient temperature. The transfection mixes were then added into the shake flasks and incubated in a shaker at 37° C., 8% CO₂, and 135 rpm, for 72 hours before harvesting. After 72 hours of incubation, cells were lysed using a lysis buffer containing 1M Tris (pH 9.5), 10% Triton X-100, 1M MgCl₂, endonuclease (e.g., BENZONASE®, DENARASE®), and 5M NaCl, and the shake flasks were incubated for 60 minutes at 37° C., 8% CO₂, and 135 rpm. Crude lysate samples were collected by centrifugation.

2 L bioreactor production: HEK293 cells were expanded for at least one passage and inoculated into a 2 L bioreactor (Millipore Mobius) containing the appropriate amount of cell culture medium prior to transfection. pH was shifted to 7.1±0.1 pre-transfection and cells were transfected at a density of 1.8E6 to 2.4E6 cells/mL (for Examples 4-8) or 3.6E6 to 5E6 cells/mL (for Examples 9-11). Transfection mixes were prepared by mixing calculated volumes of vector(s), OptiPro SFM media, and polyethylenimine (PEI), all at ambient temperature, and allowed to equilibrate for 10 min before the transfection mixes were added to the cells. Cells were harvested 69-75 hours post-transfection. Harvested cells were lysed using a lysis buffer containing 1M Tris (pH 9.5), 10% Triton X-100, 1M MgCl₂, endonuclease (e.g., BENZONASE®, DENARASE®), and 5M NaCl. Appropriate volumes of lysis buffer were added to the bioreactor, and the cells were incubated for 120 min at 37° C., and 283 rpm. Crude lysate samples were collected following centrifugation to remove cellular debris.

Vector genome productivity in number of vector genomes per cell (vg/cell) was determined by droplet digital PCR (ddPCR) by standard methods using primer/probe sets specific to the transgene payload of the vector comprising the transgene (i.e., transgene vector). Vector genome productivity in number of vector genomes per liter (vg/L) was determined by droplet digital PCR (ddPCR) by standard methods using primer/probe sets specific to the transgene payload of the vector comprising the transgene (i.e., transgene vector). The number of capsids per cell was determined using enzyme-linked immunosorbent assays (ELISAs) by standard methods with an immobilized antibody directed against an epitope of the capsid as encoded by the vector comprising the Cap sequences. Percentage of intact vector genomes (i.e., percentage of full capsids) was calculated by dividing the vector genome productivity determined by ddPCR by the number of capsids per cell determined by ELISA (in Examples 2-4), or determined by analytical ultracentrifugation sedimentation velocity (AUC) analysis (in Example 5).

### Example 2: Comparison Between Dual and Triple Transfection Systems

An initial small-scale production, proof-of-concept study was performed to assess the utility of a dual vector transfection system with respect to its vector genome (VG) productivity, and the percentage of intact vector genomes that could be obtained as compared to a triple transfection system. Transfection conditions were set up according to those set forth in Table 1.

TABLE 1
Transfection Conditions

| | | | | | | PEI: |
|---|---|---|---|---|---|---|
| | Transfection | Vectors | | | Vector | Ratio |
| Condition | System | Transgene | Rep/Cap | Helper | Ratio | DNA |
| 1 | Triple (control) | V1 | V2 | V3 | 1:1.5:2 | 2:1 |

| | | | | | |
|---|---|---|---|---|---|
| 2 | Dual | V4 | V3 | 1:3 | 2:1 |

As set forth in Table 1, the dual vector transfection system comprised a first V4 vector, and a second V3 vector. The triple vector transfection system comprises vectors V1, V2, and V3. In Table 1, the vector ratios were based on mass. Elements contained within the various vectors are set forth in Table 2.

Transfection mixtures for each transfection condition were prepared in an appropriately sized conical tube by adding calculated volumes of vector(s), OptiPro media, and polyethylenimine (PEI), all at ambient temperature. Transfection mixtures were added to cells at a concentration of 1 μg DNA/1E6 cells. Shake flasks were incubated for 72 hours before harvesting. At harvest, cells were lysed, and crude lysate samples were collected following centrifugation to remove cell debris for subsequent droplet digital PCR (ddPCR) and capsid analysis by ELISA.

TABLE 2
Vector Elements

| | Element (SEQ ID NO:) | | | | | |
|---|---|---|---|---|---|---|
| | | rAAV | | | Rep-Cap | Helper |
| Vector | Transgene | Genome | Rep | Cap | Element | Element |
| V1 | 74 | 75 | — | — | — | — |
| V2 | — | — | 50 | 72 | 73 | — |
| V3 | — | — | — | — | — | 63 |
| V4 | 74 | 75 | 50 | 72 | 73 | — |
| V5 | 74 | 70 | — | — | — | — |
| V6 | 74 | 70 | 50 | 72 | 73 | — |
| V7 | — | — | 50 | 72 | 73 | 63 |
| V8 | 79 | 80 | 50 | 72 | 73 | — |
| V9 | 79 | 80 | — | — | — | — |
| V10 | 81 | 82 | 50 | 72 | 73 | — |
| V11 | 81 | 82 | — | — | — | — |
| V12 | 74 | 78 | 50 | 72 | 73 | — |
| V13 | 74 | 78 | — | — | — | — |
| V14 | 83 | 84 | 50 | 76 | 77 | — |
| V15 | 83 | 84 | — | — | — | — |
| V16 | — | — | 50 | 76 | 77 | — |
| V17 | 79 | 66 | — | — | — | — |
| V18 | 79 | 66 | 50 | 72 | 73 | — |
| V19 | 89 | 90 | — | — | — | — |
| V20 | 89 | 90 | 50 | 72 | 73 | — |
| V21 | 89 | 90 | — | — | — | 63 |
| V22 | 74 | 75 | 50 | 91 | 92 | — |
| V23 | — | — | 50 | 91 | 92 | — |
| V24 | — | — | 50 | 93 | 94 | — |
| V25 | 74 | 75 | 50 | 93 | 94 | — |
| V26 | — | — | 50 | 95 | 96 | — |
| V27 | 74 | 75 | 50 | 95 | 96 | — |
| V28 | — | — | 50 | 97 | 98 | — |
| V29 | 74 | 75 | 50 | 97 | 98 | — |
| V30 | — | — | 50 | 99 | 100 | — |
| V31 | 74 | 75 | 50 | 99 | 100 | — |
| V32 | — | — | 50 | 101 | 102 | — |
| V33 | 74 | 75 | 50 | 101 | 102 | — |
| V34 | — | — | 50 | 103 | 104 | — |
| V35 | 74 | 75 | 50 | 103 | 104 | — |
| V36 | — | — | 50 | 105 | 106 | — |
| V37 | 74 | 75 | 50 | 105 | 106 | — |

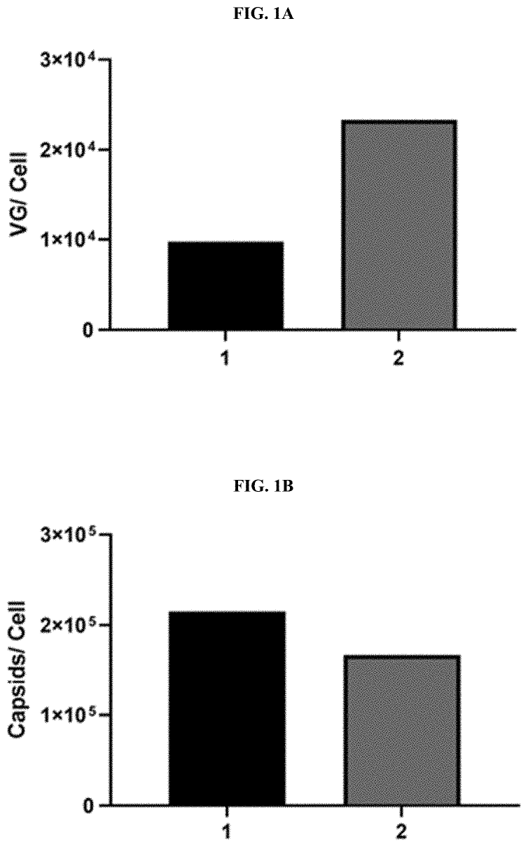

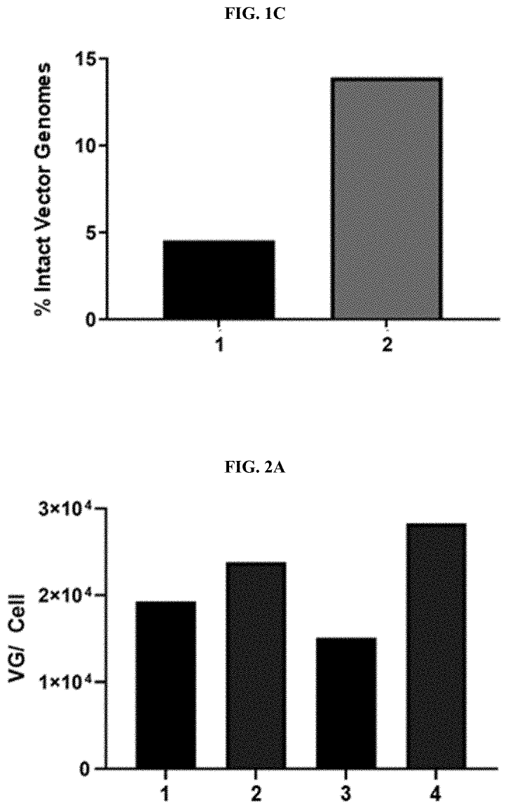

FIGS. 1A-1C show the VG productivity (FIG. 1A), capsid productivity (FIG. 1B), and percentage of intact vector genomes (FIG. 1C) obtained from production using dual and triple transfection systems. As shown in FIGS. 1A and 1C, VG productivity and percentage of intact vector genomes obtained from production using the dual vector transfection system were found to be higher than that obtained from the triple vector transfection system. These data demonstrate that using the dual vector transfection system results in an increased rAAV titer as compared with the control triple vector transfection system. The various conditions shown in FIGS. 1A-1C are set forth in Table 1.

A confirmatory experiment was performed, with additional transfection conditions to determine whether the increased VG productivity and increased percentage of intact vector genomes obtained from the dual transfection system could be replicated using a different transgene vector. Transfection conditions were set up according to those set forth in Table 3, and the elements contained within the various vectors are set forth in Table 2. In Table 2, the vector ratios were based on mass.

TABLE 3
Transfection Conditions

| | | | | | | PEI: |
|---|---|---|---|---|---|---|
| | Transfection | Vectors | | | Vector | Ratio |
| Condition | System | Transgene | Rep/Cap | Helper | Ratio | DNA |
| 1 | Triple (control) | V1 | V2 | V3 | 1:1.5:2 | 2:1 |
| 2 | Dual | V4 | | V3 | 1:3 | 2:1 |
| 3 | Triple (control) | V5 | V2 | V3 | 1:1.5:2 | 2:1 |

| | | | | | |
|---|---|---|---|---|---|
| 4 | Dual | V6 | V3 | 1:3 | 2:1 |

Transfection mixtures for each transfection condition were prepared in an appropriately sized conical tube by adding calculated volumes of vector(s), OptiPro media, and polyethylenimine (PEI), all at ambient temperature. Transfection mixtures were added to cells at a concentration of 1 μg DNA/1E6 cells. Shake flasks were incubated for 72 hours before harvesting. At harvest, cells were lysed, and crude lysate samples were collected following centrifugation to remove cell debris for subsequent droplet digital PCR (ddPCR) and capsid analysis by ELISA.

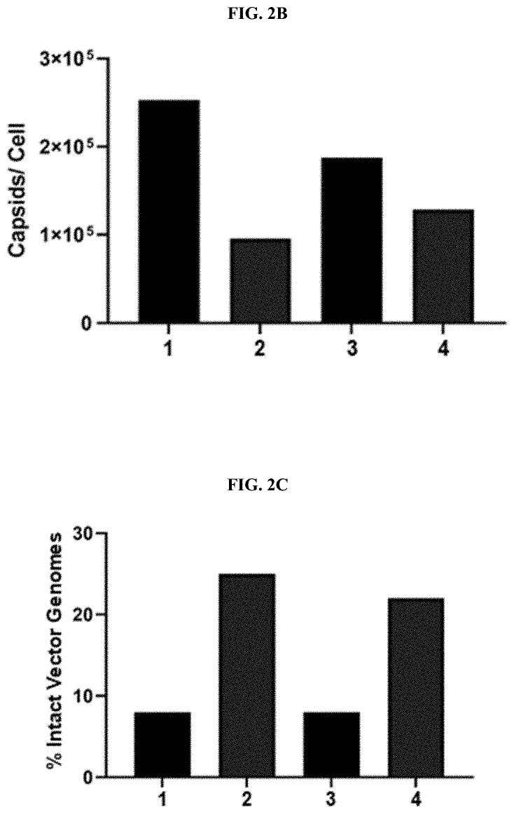

FIGS. 2A-2C show the VG productivity (FIG. 2A), capsid productivity (FIG. 2B), and percentage of intact vector genomes (FIG. 2C) obtained from production using dual and triple transfection systems. As shown in FIGS. 2A and 2C, VG productivity and percentage of intact vector genomes obtained from production using the dual vector transfection system were found to be higher than that obtained from the triple vector transfection system. The increased productivity of the dual vector transfection system was found to be consistent across at least two different transgene vectors that comprise either an editing genome comprising human genome specific homology arms (conditions 1 and 2) or an editing genome comprising mouse genome specific homology arms (conditions 3 and 4). The various conditions shown in FIGS. 2A-2C are set forth in Table 3.

Taken together, the data presented in this Example indicates the efficacy of a dual vector transfection system as compared to a triple transfection system. In particular, the dual vector transfection system increased crude lysate titers and percentage of intact vector genomes.

### Example 3: Comparison Between Dual Vector Transfection System Designs

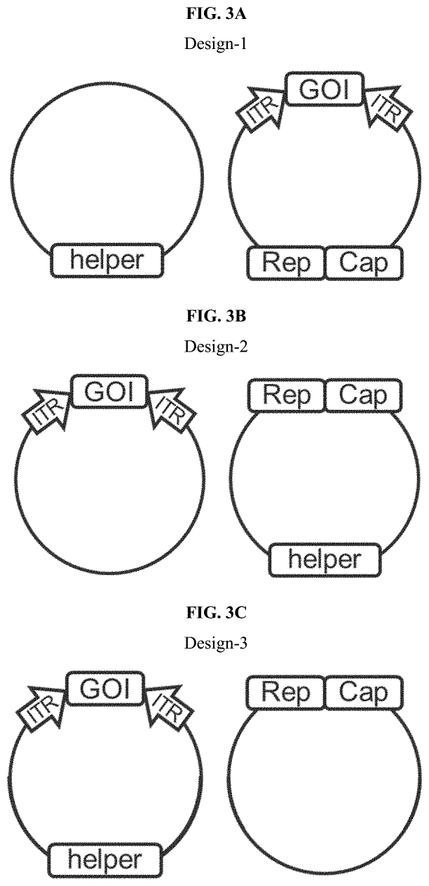

In order to investigate whether the organization of vector elements in a dual vector transfection system affects productivity, two dual vector transfection system designs were tested. The vector genome (VG) productivity and the percentage of intact vector genomes obtained from production based on each design were evaluated. Dual vector transfection system design-1 (“design-1”) and design-2 (“design-2”) differ in which vector the Rep/Cap sequence resides on with respect to the vector genome and helper sequences. FIGS. 3A-3B provide a schematic of design-1 (FIG. 3A) and design-2 (FIG. 3B). As shown, design-1 comprises a first vector comprising the Rep/Cap sequence and transgene (“GOT”), and a second vector comprising helper sequences (FIG. 3A); and design-2 comprises a first vector comprising the transgene (“GOT”), and a second vector comprising both helper and Rep/Cap sequences (FIG. 3B). Transfection conditions were set up according to those set forth in Table 4.

TABLE 4
Transfection Conditions

| | | | | | | PEI: |
|---|---|---|---|---|---|---|
| | Transfection | Vectors | | | Vector | Ratio |
| Condition | System | Transgene | Rep/Cap | Helper | Ratio | DNA |
| 1 | Dual design-1 | V4 | | V3 | 1:0.5 | 2:1 |
| 2 | Dual design-1 | V4 | | V3 | 1:1 | 2:1 |
| 3 | Dual design-1 | V4 | | V3 | 1:3 | 2:1 |
| 4 | Dual design-2 | V1 | V7 | | 1:0.5 | 2:1 |
| 5 | Dual design-2 | V1 | V7 | | 1:1 | 2:1 |
| 6 | Dual design-2 | V1 | V7 | | 1:3 | 2:1 |
| 7 | Triple (control) | V1 | V2 | V3 | 1:1:1 | 2:1 |

As set forth in Table 4, design-1 comprises a first V4 vector and a second V3 vector. Design-2 comprises a first V1 vector and a second V7 vector. VG productivity and percentage of intact vector genomes obtained from triple transfection were assessed as a control. Elements contained within the various vectors are set forth in Table 2. In Table 4, the vector ratios were based on plasmid size (i.e., molar ratios) to take into account the different sizes of the vectors when comparing dual vector transfection system designs.

Transfection mixtures for each transfection condition were prepared in an appropriately sized conical tube by adding calculated volumes of vector(s), OptiPro media, and polyethylenimine (PEI), all at ambient temperature. Transfection mixtures were added to cells at a concentration of 1 μg DNA/1E6 cells. Shake flasks were incubated for 72 hours before harvesting. At harvest, cells were lysed, and crude lysate samples were collected following centrifugation to remove cell debris for subsequent droplet digital PCR (ddPCR) and capsid analysis by ELISA.

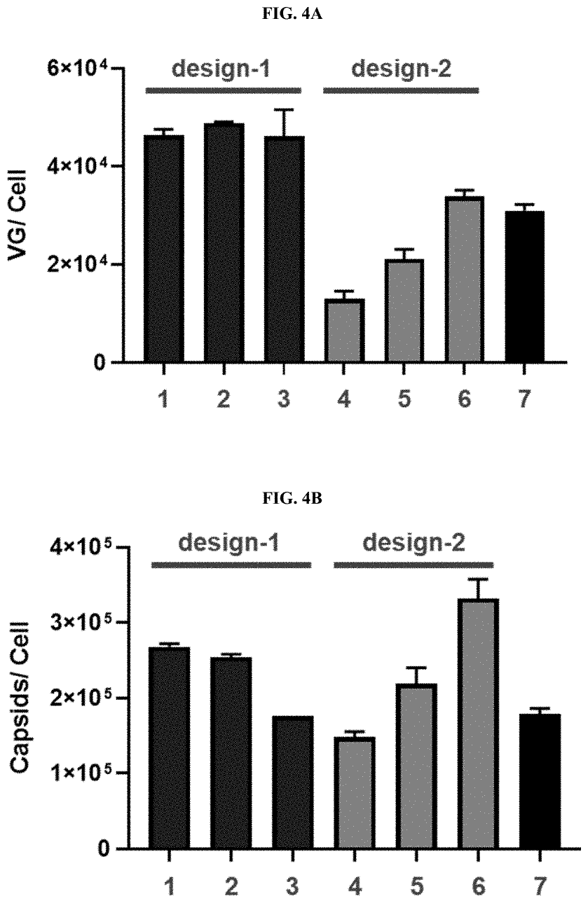

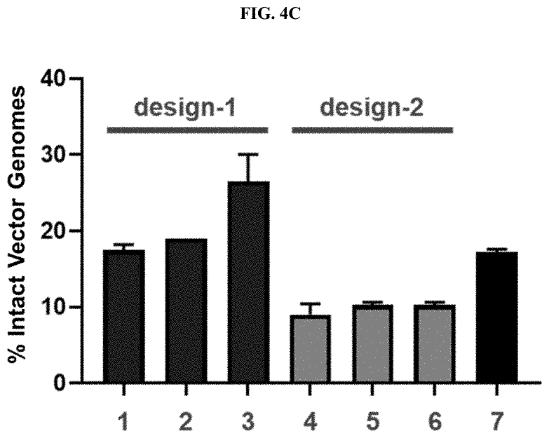

FIGS. 4A-4C show the VG productivity (FIG. 4A), capsid productivity (FIG. 4B), and percentage of intact vector genomes (FIG. 4C) obtained from production using dual and triple transfection systems. As shown in FIGS. 4A and 4C, VG productivity and percentage of intact vector genomes obtained from production using design-1 were found to be higher than that obtained from the triple transfection system. Further, as shown in FIGS. 4A and 4C, VG productivity and percentage of calculated intact vector genomes, obtained from production using design-1, were found to be higher than those obtained from production using design-2. Based on these results, design-1 was selected for further studies. The various conditions shown in FIGS. 4A-4C are set forth in Table 4.

A third dual vector transfection system design (“design-3”) was tested. The vector genome (VG) productivity and the percentage of intact vector genomes obtained from production based on each of the three designs were evaluated side by side. As discussed above, design-1 comprises a first vector comprising the Rep/Cap sequence and transgene (“GOT”), and a second vector comprising helper sequences (FIG. 3A); design-2 comprises a first vector comprising the transgene (“GOT”), and a second vector comprising both helper and Rep/Cap sequences (FIG. 3B); and design 3 comprises a first vector comprising the transgene (“GOT”) and helper sequences, and a second vector comprising the Rep/Cap sequence (FIG. 3C). Transfection conditions were set up according to those set forth in Table 5.

TABLE 5
Transfection Conditions

| | | | | | | PEI: |
|---|---|---|---|---|---|---|
| | Transfection | Vectors | | | Vector | DNA |
| Condition | System | Transgene | Rep/Cap | Helper | Ratio | Ratio |
| 1 | Dual design-1 | V20 | | V3 | 1:1.3 | 2:1 |
| 2 | Dual design-2 | V19 | V7 | | 1:3.3 | 2:1 |
| 3 | Dual design-3 | V21 | V2 | V21 | 2:1 | 2:1 |
| 4 | Triple (Control) | V19 | V2 | V3 | 1:1.4:2.3 | 2:1 |

As set forth in Table 5, design-1 comprises a first V20 vector and a second V3 vector. Design-2 comprises a first V19 vector and a second V7 vector. Design-3 comprises a first V21 vector and a second V2 vector. VG productivity and percentage of intact vector genomes obtained from triple transfection were assessed as a control. Elements contained within the various vectors are set forth in Table 2. In Table 5, the vector ratios were mass-based ratios converted from 1:1 (1:1:1) molar ratios.

Transfection mixtures for each transfection condition were prepared in an appropriately sized conical tube by adding calculated volumes of vector(s), OptiPro media, and polyethylenimine (PEI), all at ambient temperature. Transfection mixtures were added to cells at a concentration of 1 μg DNA/1E6 cells. Shake flasks were incubated for 72 hours before harvesting. At harvest, cells were lysed, and crude lysate samples were collected following centrifugation to remove cell debris for subsequent droplet digital PCR (ddPCR) and capsid analysis by ELISA.

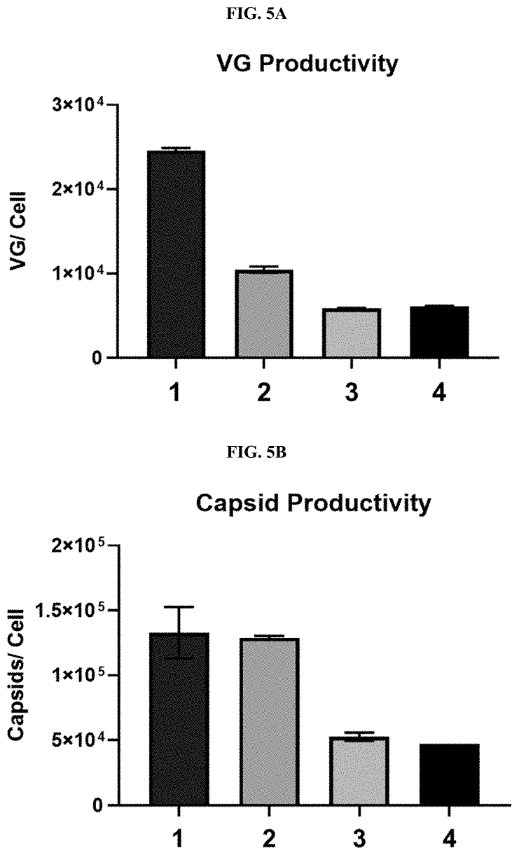

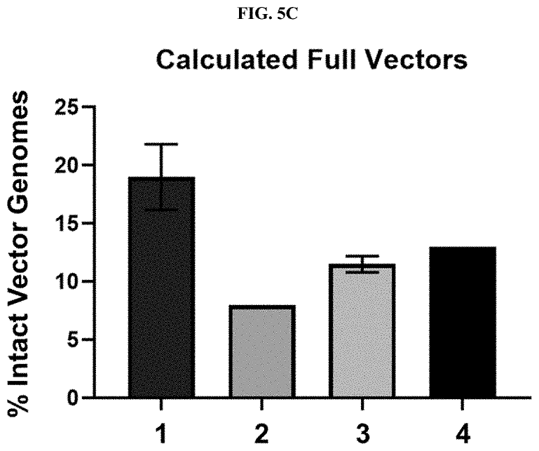

FIGS. 5A-5C show the VG productivity (FIG. 5A), capsid productivity (FIG. 5B), and percentage of intact vector genomes (FIG. 5C) obtained from production using dual and triple transfection systems. As shown in FIGS. 5A and 5C, VG productivity and percentage of intact vector genomes obtained from production using design-1 were found to be higher than that obtained from the triple transfection system. Further, as shown in FIGS. 5A and 5C, VG productivity and percentage of calculated intact vector genomes, obtained from production using design-1, were found to be higher than those obtained from production using design-2 and design-3. These data demonstrate that using the design-1 dual vector transfection system results in an increased rAAV titer as compared with the design-2 dual vector transfection system, design-3 dual vector transfection system and control triple vector transfection system. The various conditions shown in FIGS. 5A-5C are set forth in Table 5.

### Example 4: Comparison Between Dual and Triple Transfection Systems

To confirm the increased productivity of design-1 over triple transfection observed in Example 3, transfection conditions were set up to investigate whether the increased efficacy is maintained at larger scale (2 L scale), and whether increased efficacy of design-1 extends across the packaging of rAAV genomes having different transgenes and into different capsids. Transfection conditions were set up according to those set forth in Table 6. In Table 6, the vector ratios were based on mass.

TABLE 6
Transfection Conditions

| | | | | | | PEI: |
|---|---|---|---|---|---|---|
| | Transfection | Vectors | | | Vector | Ratio |
| Condition | System | Transgene | Rep/Cap | Helper | Ratio | DNA |
| 1 | Dual design-1 | V4 | | V3 | 1:3 | 2:1 |
| | Triple (control) | V1 | V2 | V3 | 1:2:2 | 2:1 |
| 2 | Dual design-1 | V8 | | V3 | 1:2 | 2:1 |
| | Dual design-1 | V8 | | V3 | 1:3 | 2:1 |
| | Dual design-1 | V8 | | V3 | 1:4 | 2:1 |
| | Triple (control) | V9 | V2 | V3 | 1:2:2 | 1.5:1 |
| 3 | Dual design-1 | V10 | | V3 | 1:2 | 2:1 |
| | Dual design-1 | V10 | | V3 | 1:3 | 2:1 |
| | Dual design-1 | V10 | | V3 | 1:4 | 2:1 |
| | Triple (control) | V11 | V2 | V3 | 1:2:2 | 1.5:1 |
| 4 | Dual design-1 | V12 | | V3 | 1:2 | 2:1 |
| | Dual design-1 | V12 | | V3 | 1:3 | 2:1 |
| | Dual design-1 | V12 | | V3 | 1:4 | 2:1 |
| | Triple (control) | V13 | V2 | V3 | 1:2:2 | 2:1 |
| 5 | Dual design-1 | V18 | | V3 | 1:2 | 2:1 |
| | Dual design-1 | V18 | | V3 | 1:3 | 2:1 |
| | Dual design-1 | V18 | | V3 | 1:4 | 2:1 |
| | Triple (control) | V17 | V2 | V3 | 1:2:2 | 1.5:1 |
| 6 | Dual design-1 | V14 | | V3 | 1:4 | 1.5:1 |
| | Triple (control) | V15 | V16 | V3 | 1:2:2 | 1.5:1 |
| 7 | Dual design-1 | V22 | | V3 | 1:1.1 | 2:1 |
| | Triple (control) | V1 | V23 | V3 | 1:1.1:1.9 | 2:1 |

As set forth in Table 6, transfection conditions 1, 2, 3, 4, 5, and 6 were set up to investigate whether increased efficacy of design-1 extends across the packaging of rAAV genomes having different transgenes. In addition to investigating the efficacy of design-1 across packaging of rAAV genomes having different transgenes, conditions 6 and 7 also assess whether the efficacy extends across the packaging of rAAV genomes into different capsids. Conditions 1-5 each utilized AAVHSCS15 capsid, condition 6 utilized AAVHSCS17 capsid, and condition 7 utilized AAV2 capsid. VG productivity and percentage of intact vector genomes obtained from triple transfection were assessed as a control. Elements contained within the various vectors are set forth in Table 2.

Transfection mixtures for each transfection condition were prepared in an appropriately sized transfer assembly by adding calculated volumes of vector(s), OptiPro media, and polyethylenimine (PEI), all at ambient temperature. Transfection mixtures were added to cells at a concentration of 1 μg DNA/1E6 cells. Cells were incubated for 72 hours before harvesting.

At harvest, cells were lysed, and crude lysate samples were collected following centrifugation to remove cell debris for subsequent droplet digital PCR (ddPCR) and capsid analysis by ELISA.

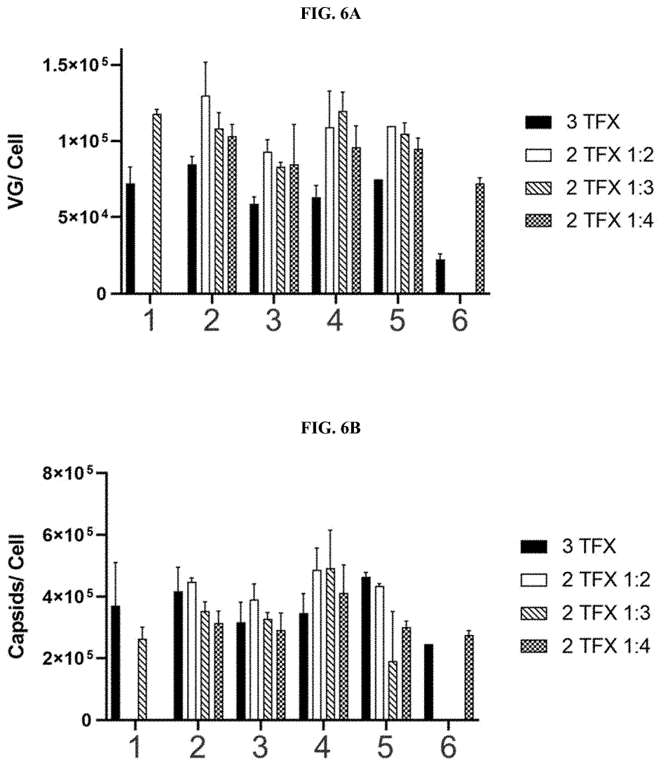

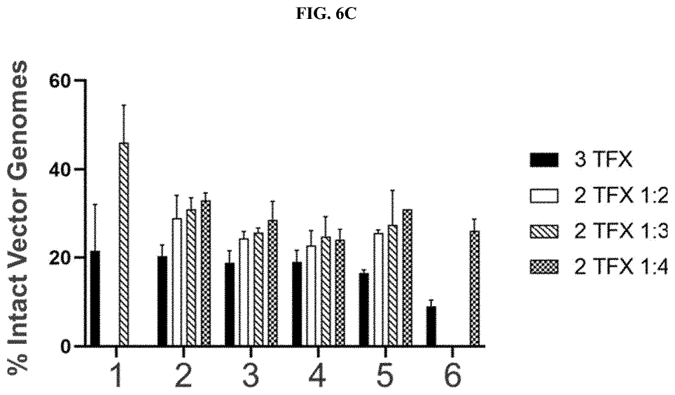

FIGS. 6A-6C show the VG productivity (FIG. 6A), capsid productivity (FIG. 6B), and percentage of intact vector genomes (FIG. 6C) obtained from production using design-1 and the control triple transfection system. As shown in FIGS. 6A and 6C, VG productivity and percentage of intact vector genomes obtained from production using design-1 were found to be higher than that obtained from the triple transfection system, in all conditions tested. Based on these results, the increased efficacy of production using design-1 over triple transfection was observed across the packaging of rAAV having different transgenes into different capsids. The increased productivity of the dual vector transfection system was found to be consistent across five different rAAV genomes, two of which comprise editing genomes (conditions 1 and 2). These data demonstrate that the increased rAAV titer obtained using the design-1 dual vector transfection system over the control triple vector transfection system extends across the packaging of rAAV having different transgenes into different capsids. The various conditions shown in FIGS. 6A-6C are set forth in Table 6.

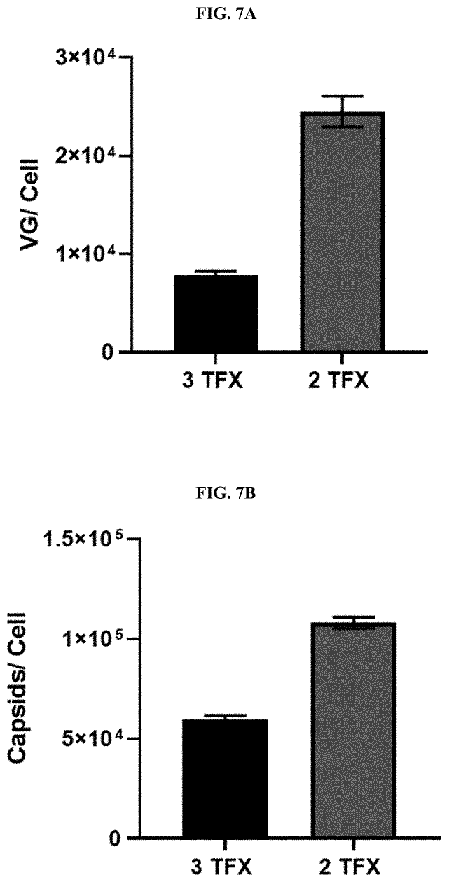

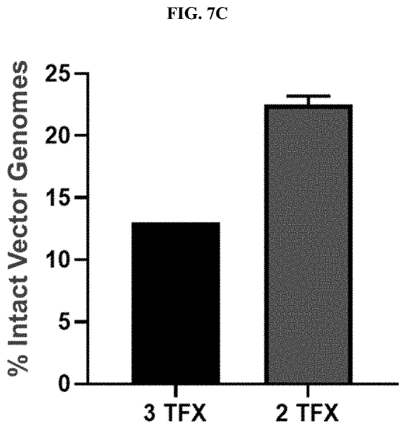

FIGS. 7A-7C show the VG productivity (FIG. 7A), capsid productivity (FIG. 7B), and percentage of intact vector genomes (FIG. 7C) obtained from production utilizing AAV2 capsid using design-1 and the control triple transfection system (condition 7). As shown in FIGS. 7A and 7C, VG productivity and percentage of intact vector genomes obtained from production utilizing AAV2 capsid using design-1 were found to be higher than that obtained from the triple transfection system. The data in FIGS. 7A-7C were generated from small-scale production studies.

In a separate experiment, it was found that design-1 was also able to produce rAAV comprising an AAVHSC13 capsid (see, U.S. Pat. No. 9,803,218, which is incorporated herein in its entirety).

These data suggest that the improvements in AAV production exhibited by the design-1 dual plasmid system (relative to triple plasmid system controls) are likely generally applicable.

### Example 5: Comparison Between Dual and Triple Transfection Systems

Examples 3 and 4 demonstrated increased VG productivity and increased percentage of intact vector genomes measured in crude lysates obtained from production using design-1, as compared to production using a control triple transfection system.

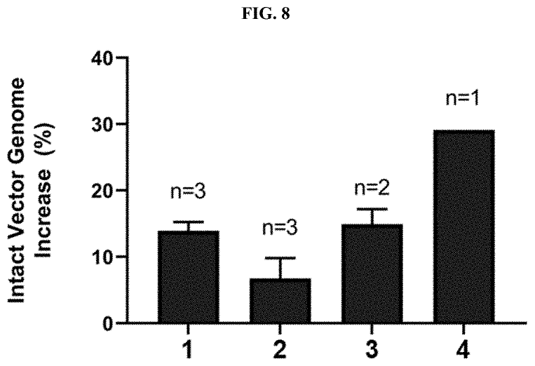

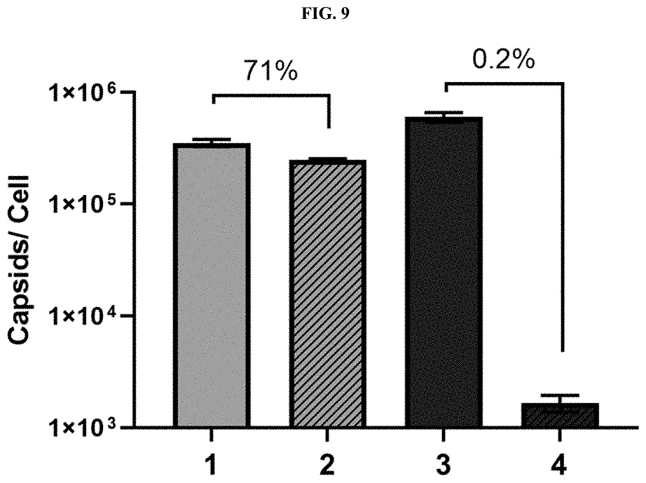

To confirm that the increased VG productivity and increased percentage of intact vector genomes were maintained post-purification, the crude lysates obtained from transfections set up according to those set forth in Table 7 were clarified and subsequently purified by affinity and anion exchange chromatography. In Table 7, except for condition 3 which was performed at 50 L scale, conditions 1, 2, and 4 were performed according to conditions 2, 3, and 5 in Table 6 (i.e., at 2 L scale), respectively. Lysates produced using different vector ratios were purified separately. Conditions 1-3 each utilized AAVHSCS15 capsid, whereas condition 4 utilized AAVHSCS17 capsid. Intact vector genomes obtained from the design-1 dual plasmid systems were expressed as a percentage increase over the amount of intact vector genomes obtained from the indicated control triple plasmid system (Table 7 and FIG. 8). In Table 7, the vector ratios were based on mass. Elements contained within the various vectors are set forth in Table 2.

TABLE 7
Transfection Conditions

| | | | | | | PEI: |
|---|---|---|---|---|---|---|
| | Transfection | Vectors | | | Vector | Ratio |
| Condition | System | Transgene | Rep/Cap | Helper | Ratio | DNA |
| 1 | Dual design-1 | V8 | | V3 | 1:2 | 2:1 |
| | Dual design-1 | V8 | | V3 | 1:3 | 2:1 |
| | Dual design-1 | V8 | | V3 | 1:4 | 2:1 |
| | Triple (control) | V9 | V2 | V3 | 1:2:2 | 1.5:1 |
| 2 | Dual design-1 | V10 | | V3 | 1:2 | 2:1 |
| | Dual design-1 | V10 | | V3 | 1:3 | 2:1 |
| | Dual design-1 | V10 | | V3 | 1:4 | 2:1 |
| | Triple (control) | V11 | V2 | V3 | 1:2:2 | 1.5:1 |
| 3 | Dual design-1 | V12 | | V3 | 1:2 | 2:1 |
| | Triple (control) | V1 | V2 | V3 | 1:1.5:2 | 2:1 |
| 4 | Dual design-1 | V14 | | V3 | 1:4 | 1.5:1 |
| | Triple (control) | V15 | V16 | V3 | 1:2:2 | 1.5:1 |

The data depicted in FIG. 8 is based on analytical ultracentrifugation sedimentation velocity (AUC) analysis, a method used to quantify macromolecules based on sedimentation coefficients. AUC was used to determine the percentage of intact vector genomes and capsids that lack a vector genome produced by each design-1 dual plasmid system, relative to the corresponding triple plasmid system control. In FIG. 8, for Conditions 1 and 2, AUC was performed on purified vectors obtained from each of the design-1 vector ratios (i.e., 1:2, 1:3, and 1:4 ratios shown in Table 7) to determine the number of intact vector genomes, and then averaged and presented as a percent increase relative to the corresponding triple plasmid system control. As shown in FIG. 8, an increase in the number of intact vector genomes was obtained for each of the four design-1 dual plasmid systems tested (relative to the number of intact vector genomes obtained from the corresponding triple plasmid system control). These data suggest that the improvements in AAV production exhibited by the design-1 dual plasmid system (relative to triple plasmid system controls) are likely generally applicable and scalable.

### Example 6: Capsid Background Expression in Dual Transfection Systems

In an effort to elucidate the reason why design-1 outperformed other dual plasmid transfection system designs, the level of background capsid expression was determined in design-1 and compared to the level of background capsid expression in design-2. Transfection conditions were set up according to those set forth in Table 8. In Table 8, the vector ratios were based on mass.

TABLE 8
Transfection Conditions

| | | | | | | PEI: |
|---|---|---|---|---|---|---|
| | Transfection | Vectors | | | Vector | Ratio |
| Condition | System | Transgene | Rep/Cap | Helper | Ratio | DNA |

| | | | | | |
|---|---|---|---|---|---|
| 1 | Dual design-2 | V1 | V7 | 1:2.4 | 2:1 |
| 2 | Dual design-2 | — | V7 | 1 | 2:1 |
| | (Rep/Cap | | | | |
| | containing | | | | |
| | vector only) | | | | |

| | | | | | |
|---|---|---|---|---|---|
| 3 | Dual design-1 | V4 | V3 | 1:1.1 | 2:1 |
| 4 | Dual design-1 | — | V3 | 1 | 2:1 |
| | (Rep/Cap | | | | |
| | containing | | | | |
| | vector only) | | | | |

As shown in Table 8, design-1 and design-2 were tested together with only the Rep/Cap containing vector for each respective dual design. The same amount of Rep/Cap containing vector was used alone (e.g., Conditions 2 and 4) or as a vector in a dual design (e.g., Conditions 1 and 3).

It was found that the level of background capsid generation from design-2 (transfection of only vector V7; Condition 2) was the same as the level of background capsids generated from dual transfection of design-2 (transfection of both vectors V1 and V7; Condition 1) (FIG. 9). As shown in FIG. 9, background capsid generation from design-1 was less than 1% of the level of background capsids generated from dual transfection of design-1 (comparing Condition 4 to Condition 3).

### Example 7: Large-Scale Production and Quality Assessment of AAV from Dual and Triple Transfection Systems

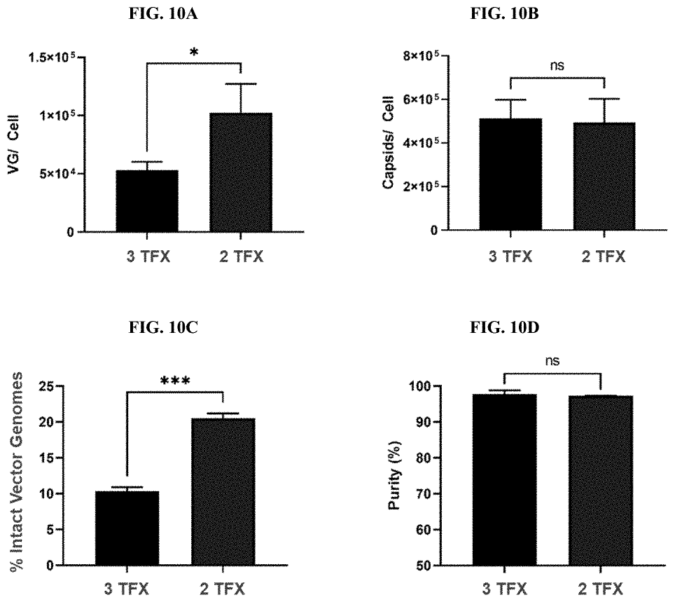

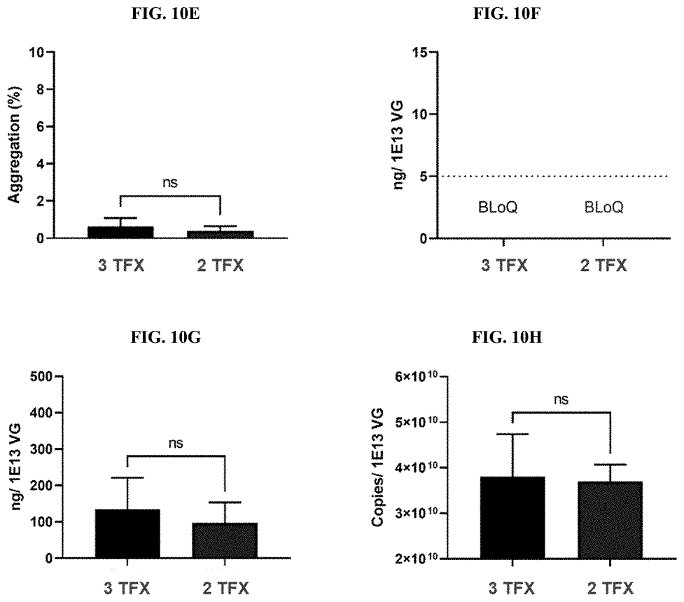

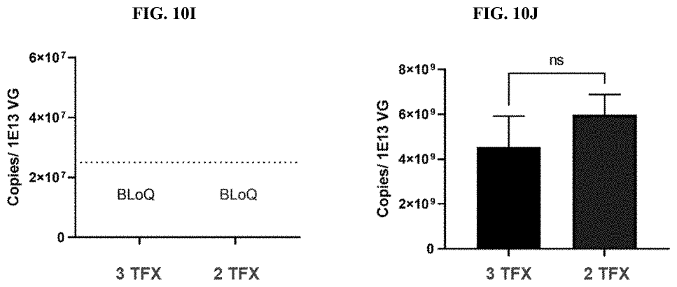

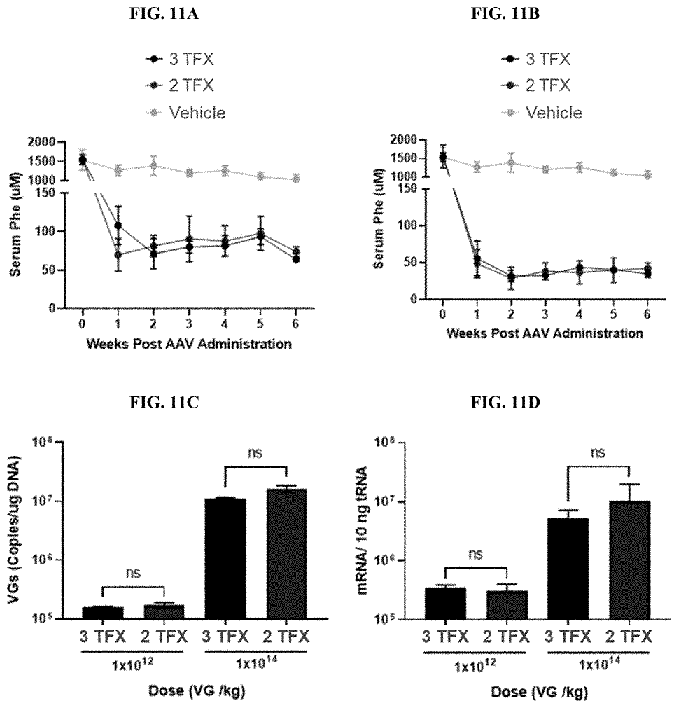

To investigate whether the improved productivity of design-1 is maintained at larger scale production, Condition 4 in Table 6 at a vector ratio of 1:2 for design-1 was repeated at 50 L bioreactor scale. Consistent with the trends at shake flask and 2 L bioreactor scale, the results from the 50 L bioreactors demonstrated an almost 2-fold increase in VG productivity, comparable capsid production and a doubling in the calculated intact vector genomes in the crude lysate obtained from design-1 (“2 TFX”) compared to crude lysate obtained from a triple transfection system (“3 TFX”; see, Table 6 for conditions of triple transfection control) (FIGS. 10A-10C). These data demonstrate that increased rAAV titer obtained using the design-1 dual vector transfection system as compared with the control triple vector transfection system is maintained at larger scale production.

Various analytical methods were used to characterize product quality of AAV vectors obtained from design-1 and a triple transfection system (FIGS. 10D-10J). As shown, percent purity (FIG. 10D), percent aggregation (FIG. 10E), and level of residual host cell protein (FIG. 10F; BLoQ means below limit of quantification) all remained consistent regardless of transfection method. No deviations were found in the amount of residual host cell DNA (FIG. 10G), Rep/Cap (FIG. 10H), Ela (FIG. 10I), and Helper sequences (FIG. 10J) packaged in purified AAV vectors obtained from design-1 compared to those obtained from the triple transfection system.

### Example 8: Bioactivity of AAV Vectors Obtained from Dual and Triple Transfection Systems

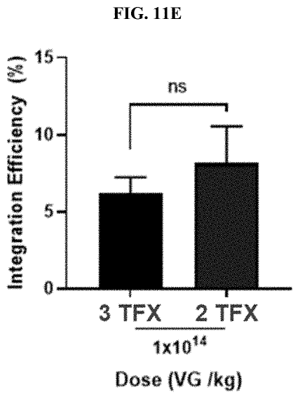

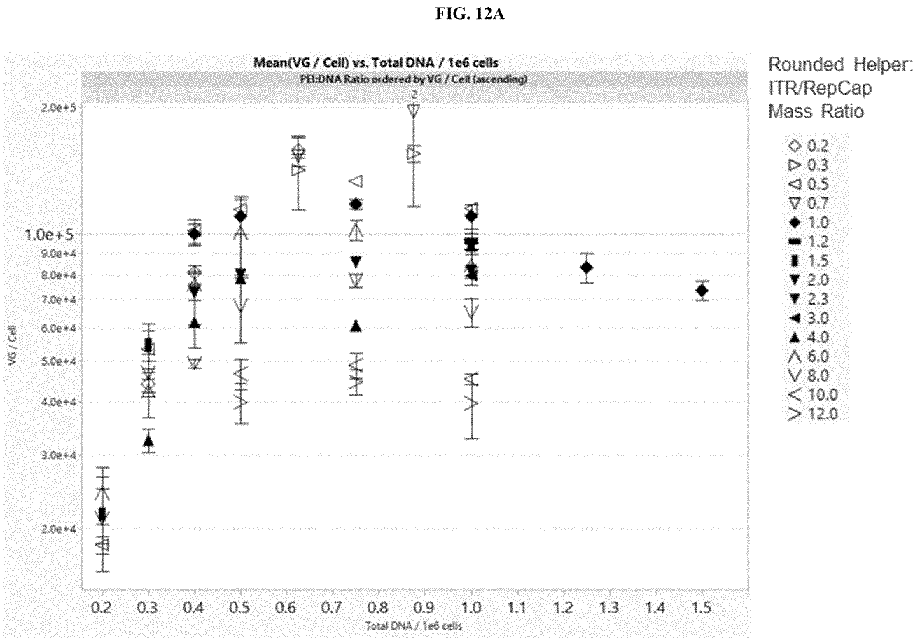

To ensure product comparability between AAV vectors obtained from design-1 and AAV vectors obtained from a triple transfection system, AAV vectors obtained from Condition 5 in Table 6 at a vector ratio of 1:4 for design 1, and the associated triple transfection control were purified and assessed for in vivo bioactivity. The rAAV genomes comprise an editing genome expressing phenylalanine hydroxylase (PAH) under the control of a liver specific promoter flanked by murine-specific homology arms. AAV vectors obtained from design-1 and from the triple transfection system were injected into Pah^{enu2} mice, a model displaying several features of classical phenylketonuria. Two doses were evaluated as well as a vehicle-only control group. Weekly serum samples were taken and analyzed for levels of phenylalanine (Phe). As shown in FIGS. 11A and 11B, at both doses of 1E12 VG/kg (FIG. 11A) and 1E14 VG/kg (FIG. 11B), the bioactivity, as indicated by a reduction in serum Phe levels post-dosing, of AAV vectors obtained from design-1 and from the triple transfection system was indistinguishable across a six-week period. Furthermore, at six weeks, quantification of vector genomes in the liver and PAH mRNA expression showed a dose dependent increase in VG transduction and transgene expression but no significant differences between design-1 and triple transfection groups at each dose (FIGS. 11C and 11D). Quantification of on-target integration was completed at the 1E14 VG/kg dose, and demonstrated comparable integration efficiencies for AAV vectors produced from either design-1 or the triple transfection system (FIG. 11E).

### Example 9: Optimization of Vector Ratios

To investigate whether there is an optimal vector ratio that results in improved productivity, various design-1 vector ratios were tested. Transfections were set up as described in Example 1 for small scale production.

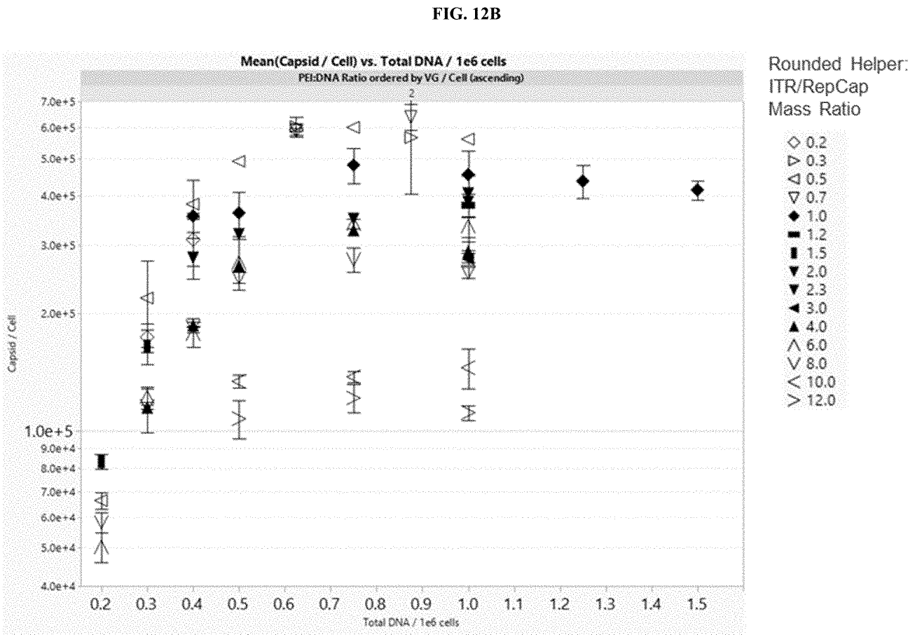

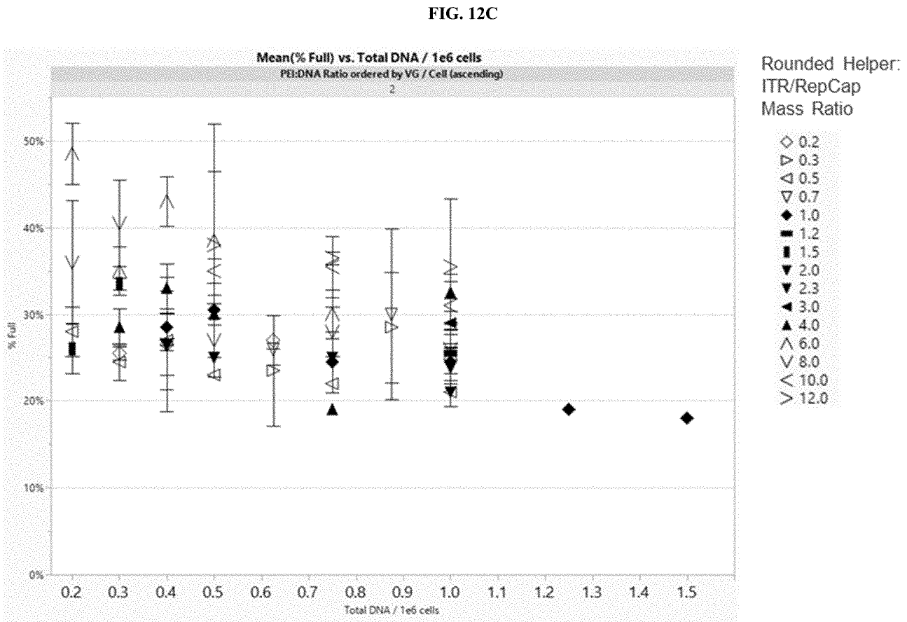

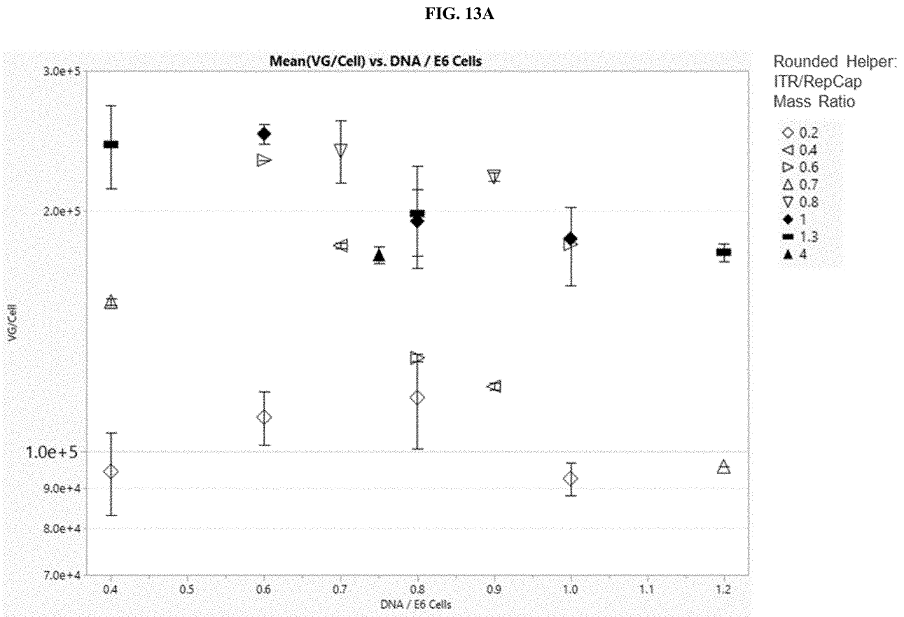

FIGS. 12A-12C show the VG productivity (FIG. 12A), capsid productivity (FIG. 12B), and percentage of intact vector genomes (FIG. 12C) obtained from production under condition 1 that tested the indicated V3:V12 vector ratios, at various levels of total DNA transfected (x-axis). Elements contained within V3 and V12 are set forth in Table 2. As shown in FIGS. 12A-12C, improved VG and capsid productivity was achieved at V3:V12 vector ratios of 1:0.3 to 1:1, using 0.6 to 1 μg of total DNA transfected per 1E6 of cells.

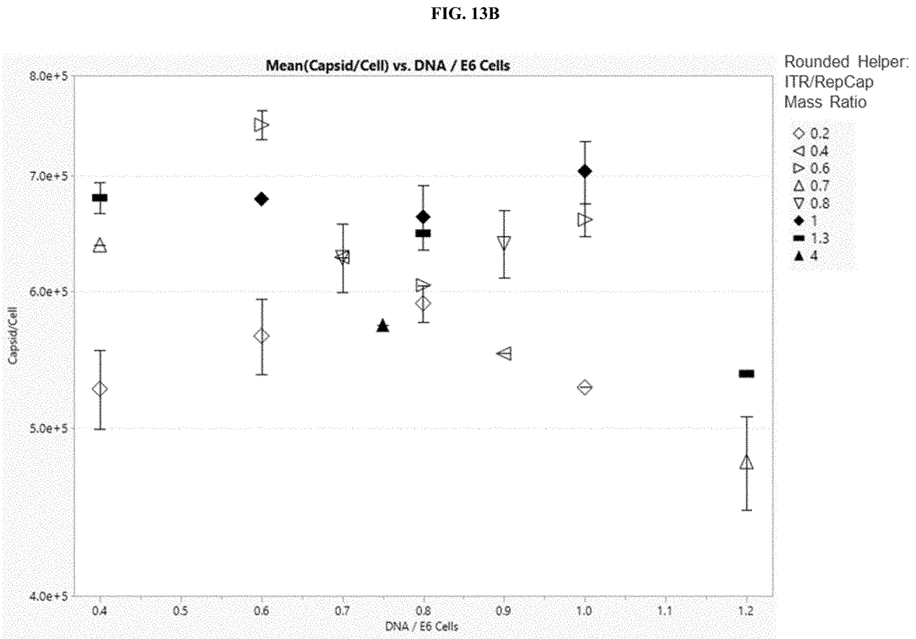

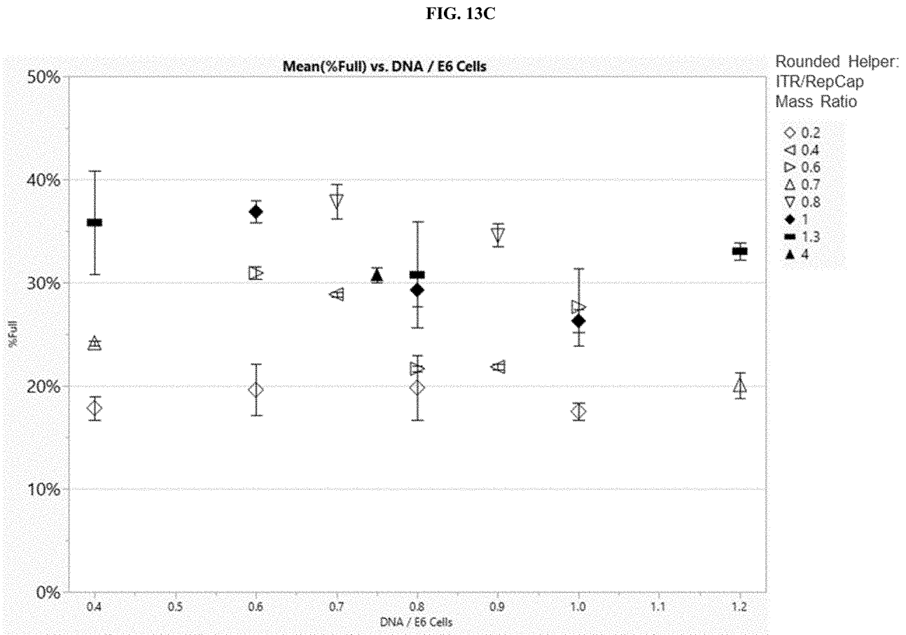

FIGS. 13A-13C show the VG productivity (FIG. 13A), capsid productivity (FIG. 13B), and percentage of intact vector genomes (FIG. 13C) obtained from production under condition 2 that tested the indicated V3:V8 ratios, at various levels of total DNA transfected (x-axis). Elements contained within V3 and V8 are set forth in Table 2. As shown in FIGS. 13A-13C, improved VG and capsid productivity was achieved at V3:V8 vector ratios of 1:0.6 to 1:1, using 0.6 to 1 μg of total DNA transfected per 1E6 of cells. These data demonstrate that an increased rAAV titer is achieved using these vector ratios and levels of total DNA transfected.

### Example 10: Assessment of Multiple Capsid Serotypes Using Dual Plasmid Transfection

To investigate whether the improved productivity of design-1 is maintained across other AAV capsid serotypes, AAV vectors produced from either design-1 or the triple transfection system were tested utilizing AAV capsid serotypes AAV1, AAV2, AAV5, AAV6, AAV8, AAV9, AAVrh10 and AAVrh74. Transfections were set up as described in Example 1 for 2 L bioreactor production. Transfection conditions were set up according to those set forth in Table 9.

TABLE 9
Transfection Conditions

| | | | | Plasmid Ratio | |
|---|---|---|---|---|---|
| Serotype/ | | | | (ITR:RepCap: | |
| Transfection | | | | Helper/ITR + | PEI:DNA |
| System | ITR | RepCap | Helper | RepCap: Helper) | Ratio |
| AAV1 Triple | V1 | V24 | V3 | 1:2:2 | 1:1.5 |
| AAV1 Dual | V25 | | V3 | 1:4 | 1:2 |
| AAV2 Triple | V1 | V23 | V3 | 1:2:2 | 1:1.5 |
| AAV2 Dual | V22 | | V3 | 1:4 | 1:2 |
| AAV5 Triple | V1 | V26 | V3 | 1:2:2 | 1:1.5 |
| AAV5 Dual | V27 | | V3 | 1:4 | 1:2 |
| AAV6 Triple | V1 | V28 | V3 | 1:2:2 | 1:1.5 |
| AAV6 Dual | V29 | | V3 | 1:4 | 1:2 |
| AAV8 Triple | V1 | V30 | V3 | 1:2:2 | 1:1.5 |
| AAV8 Dual | V31 | | V3 | 1:4 | 1:2 |
| AAV9 Triple | V1 | V32 | V3 | 1:2:2 | 1:1.5 |
| AAV9 Dual | V33 | | V3 | 1:4 | 1:2 |
| AAVrh10 Triple | V1 | V34 | V3 | 1:2:2 | 1:1.5 |
| AAVrh10 Dual | V35 | | V3 | 1:4 | 1:2 |
| AAVrh74 Triple | V1 | V36 | V3 | 1:2:2 | 1:1.5 |

| | | | | |
|---|---|---|---|---|
| AAVrh74 Dual | V37 | V3 | 1:4 | 1:2 |

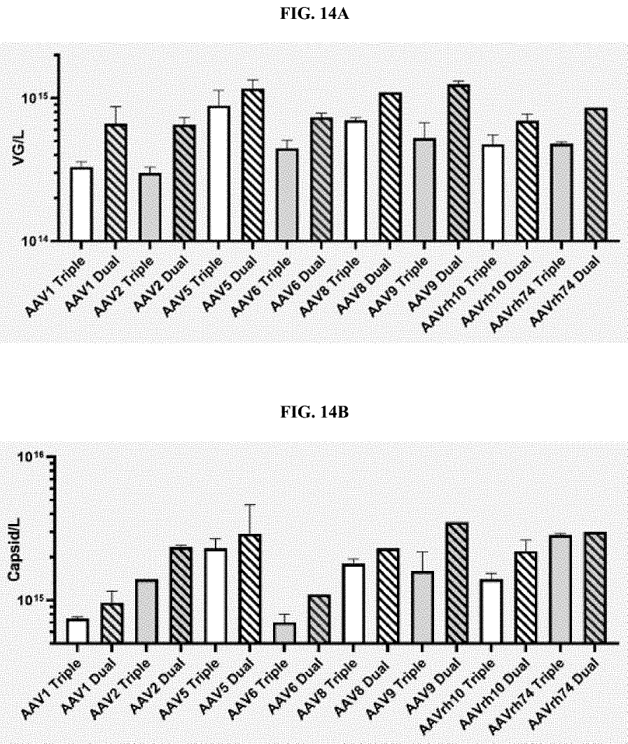

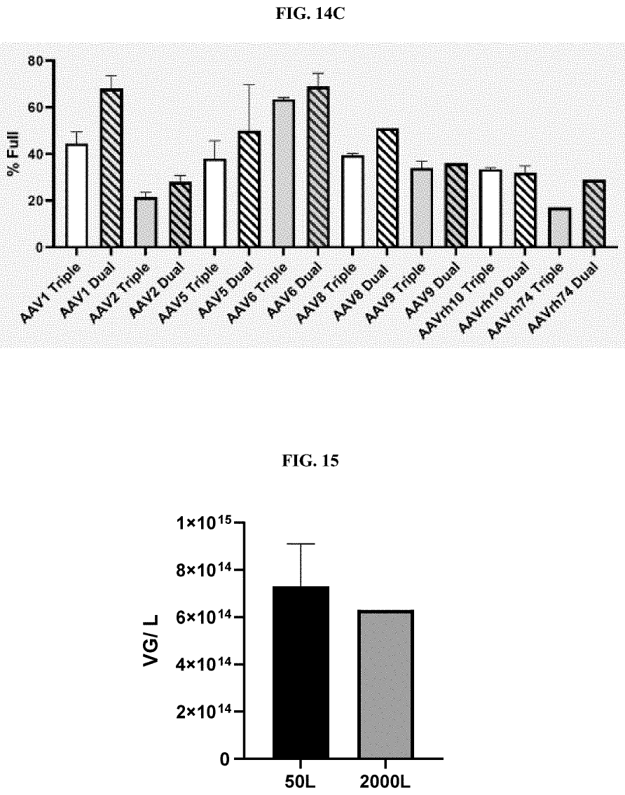

FIGS. 14A-14C show the VG productivity (FIG. 14A), capsid productivity (FIG. 14B) and percentage of intact vector genomes (FIG. 14C) obtained from production under the conditions set forth in Table 9. As shown in FIG. 14A, improved VG productivity obtained from production using design-1 relative to the corresponding triple transfection system control is maintained across all tested AAV capsid serotypes. As shown in FIG. 14B capsid productivity obtained from production using design-1 relative to the corresponding triple transfection system control is either improved or maintained. As shown in FIG. 14C, the percentage of intact vector genomes obtained from production using design-1 relative to the corresponding triple transfection system control is either improved or maintained. These data demonstrate that increased rAAV titer obtained using the design-1 dual vector transfection system as compared with the control triple vector transfection system extends across different AAV capsid serotypes.

### Example 11: Dual Plasmid Scalability to 2000 L

Example 7 showed that improved productivity of design-1 is maintained at 50 L bioreactor scale. The results from the 50 L bioreactors demonstrated an almost 2-fold increase in VG productivity in the crude lysate obtained from design-1 compared to the crude lysate obtained from a triple transfection system control.

To investigate whether the improved VG productivity of design-1 is scalable, productivity at 50 L bioreactor scale was compared with productivity at 2000 L bioreactor scale. Transfections were set up as described in Example 1 for 2 L bioreactor production, except that cells were inoculated into 50 L and 2000 L bioreactors. Cells were transfected at a density of 3.6E6 to 5E6 cells/mL. Transfection conditions for the 50 L bioreactor and 2000 L bioreactor were set up according to those set forth in Table 10.

TABLE 10
Transfection Conditions

| | | | | |
|---|---|---|---|---|
| ITR | RepCap | Helper | Plasmid Ratio | PEI:DNA Ratio |

| | | | |
|---|---|---|---|
| V8 | V3 | 1:4 | 1:2 |

FIG. 15 shows that 50 L and 2000 L bioreactor scales achieve comparable VG productivity. These data demonstrate the scalability of the design-1 dual plasmid transfection system.

Further embodiments of the invention are set out in the following clauses:

1. A first nucleic acid vector comprising: a first nucleotide sequence encoding an AAV Rep protein; a second nucleotide sequence comprising a recombinant AAV (rAAV) genome comprising a transgene; and a third nucleotide sequence encoding an AAV capsid protein, wherein the nucleic acid vector does not comprise a helper virus gene.

2. The nucleic acid vector of clause 1, comprising from 5′ to 3′: the first nucleotide sequence encoding an AAV Rep protein; the second nucleotide sequence comprising a recombinant AAV (rAAV) genome comprising a transgene; and the third nucleotide sequence encoding an AAV capsid protein, wherein the nucleic acid vector does not comprise a helper virus gene.

3. The nucleic acid vector of clause 1, comprising from 5′ to 3′: the first nucleotide sequence encoding an AAV Rep protein; the second nucleotide sequence comprising a recombinant AAV (rAAV) genome comprising a transgene; and the third nucleotide sequence encoding an AAV capsid protein.

4. The nucleic acid vector of any one of clauses 1-3, wherein the nucleic acid vector is a DNA plasmid or a DNA minimal vector.

5. A recombinant AAV (rAAV) packaging system, comprising: (i) a first nucleic acid vector comprising: a first nucleotide sequence encoding an AAV Rep protein; a second nucleotide sequence comprising a recombinant AAV (rAAV) genome comprising a transgene; and a third nucleotide sequence encoding an AAV capsid protein, and (ii) a second nucleic acid vector comprising a helper virus gene.

6. The packaging system of clause 5, wherein the first nucleic acid vector comprises from 5′ to 3′: the first nucleotide sequence encoding an AAV Rep protein; the second nucleotide sequence comprising a recombinant AAV (rAAV) genome comprising a transgene; and the third nucleotide sequence encoding an AAV capsid protein.

7. The packaging system of clause 5 or 6, wherein the first nucleic acid vector is a DNA plasmid or DNA minimal vector.

8. The packaging system of any one of clauses 5-7, wherein the second nucleic acid vector is a DNA plasmid or DNA minimal vector.

9. The nucleic acid vector or packaging system of any one of clauses 1-8, wherein the transgene encodes a polypeptide.

10. The nucleic acid vector or packaging system of any one of clauses 1-8, wherein the transgene encodes an miRNA, shRNA, siRNA, antisense RNA, gRNA, antagomir, miRNA sponge, RNA aptazyme, RNA aptamer, lncRNA, ribozyme, or mRNA.

11. The nucleic acid vector or packaging system of any one of clauses 1-8, wherein the transgene encodes a protein selected from the group consisting of phenylalanine hydroxylase (PAH), glucose-6-phosphatase (G6Pase), iduronate-2-sulfatase (I2S), arylsulfatase A (ARSA), and frataxin (FXN).

12. The nucleic acid vector or packaging system of any preceding clause, wherein the rAAV genome further comprises a transcriptional regulatory element operably linked to the transgene.

13. The nucleic acid vector or packaging system of clause 12, wherein the transcriptional regulatory element comprises a promoter element and/or an intron element.

14. The nucleic acid vector or packaging system of any preceding clause, wherein the rAAV genome further comprises a polyadenylation sequence.

15. The nucleic acid vector or packaging system of clause 14, wherein the polyadenylation sequence is 3′ to the transgene.

16. The nucleic acid vector or packaging system of any preceding clause, wherein the rAAV genome comprises a nucleotide sequence that is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 71, 85, 86, 87, or 88.

17. The nucleic acid vector or packaging system of any preceding clause, wherein the rAAV genome further comprises a 5′ inverted terminal repeat (5′ ITR) nucleotide sequence 5′ of the transgene, and a 3′ inverted terminal repeat (3′ ITR) nucleotide sequence 3′ of the transgene.

18. The nucleic acid vector or packaging system of clause 17, wherein the 5′ ITR nucleotide sequence is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 39, 41, or 42, and/or the 3′ ITR nucleotide sequence is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 40, 43, or 44.

19. The nucleic acid vector or packaging system of any preceding clause, wherein the rAAV genome comprises a nucleotide sequence that is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 75, 78, 80, 82, or 84.

20. The nucleic acid vector or packaging system of any preceding clause, wherein the AAV Rep protein is a wild-type Rep protein or a variant thereof.

21. The nucleic acid vector or packaging system of any preceding clause, wherein the AAV Rep protein is an AAV2 Rep protein or a variant thereof.

22. The nucleic acid vector or packaging system of any preceding clause, wherein the first nucleotide sequence further comprises a transcriptional regulatory element operably linked to the AAV Rep protein coding sequence.

23. The nucleic acid vector or packaging system of clause 22, wherein the transcriptional regulatory element comprises a promoter selected from the group consisting of a constitutive promoter, an inducible promoter, or a native promoter.

24. The nucleic acid vector or packaging system of clause 23, wherein the promoter is selected from the group consisting of a P5 promoter, a P19 promoter, a metallothionine (MT) promoter, a mouse mammary tumor virus (MMTV) promoter, a T7 promoter, an ecdysone insect promoter, a tetracycline-repressible promoter, a tetracycline-inducible promoter, an RU486-inducible promoter, and a rapamycin-inducible promoter.

25. The nucleic acid vector or packaging system of any preceding clause, wherein the AAV capsid protein is selected from the group consisting of AAV1, AAV2, AAV3, AAV4, AAV5, AAV6, AAV7, AAV8, AAV9, AAVrh10, AAVRh32.33, AAVrh74, AAV-DJ, AAV-LK03, NP59, VOY101, VOY201, VOY701, VOY801, VOY1101, AAVPHP.N, AAVPHP.A, AAVPHP.B, PHP.B2, PHP.B3, G2A3, G2B4, G2B5, and PHP.S.

26. The nucleic acid vector or packaging system of any preceding clause, wherein the AAV capsid protein comprises an amino acid sequence that is at least 85% identical to the amino acid sequence of amino acids 203-736 of SEQ ID NO: 1, 2, 3, 4, 5, 6, 7, 8, 9, 10, 11, 12, 13, 15, 16, or 17.

27. The nucleic acid vector or packaging system of clause 26, wherein: the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G.

28. The nucleic acid vector or packaging system of clause 27, wherein: (a) the amino acid in the capsid protein corresponding to amino acid 626 of SEQ ID NO: 16 is G, and the amino acid in the capsid protein corresponding to amino acid 718 of SEQ ID NO: 16 is G; (b) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M; (c) the amino acid in the capsid protein corresponding to amino acid 505 of SEQ ID NO: 16 is R, and the amino acid in the capsid protein corresponding to amino acid 687 of SEQ ID NO: 16 is R; (d) the amino acid in the capsid protein corresponding to amino acid 346 of SEQ ID NO: 16 is A, and the amino acid in the capsid protein corresponding to amino acid 505 of SEQ ID NO: 16 is R; or (e) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C.

29. The nucleic acid vector or packaging system of clause 27, wherein the AAV capsid protein comprises the amino acid sequence of amino acids 203-736 of SEQ ID NO: 1, 2, 3, 4, 5, 6, 7, 8, 9, 10, 11, 12, 13, 15, 16, or 17.

30. The nucleic acid vector or packaging system of any preceding clause, wherein the AAV capsid protein comprises an amino acid sequence that is at least 85% identical to the amino acid sequence of amino acids 138-736 of SEQ ID NO: 1, 2, 3, 4, 5, 6, 7, 8, 9, 10, 11, 12, 13, 15, 16, or 17.

31. The nucleic acid vector or packaging system of clause 30, wherein: the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G.

32. The nucleic acid vector or packaging system of clause 31, wherein: (a) the amino acid in the capsid protein corresponding to amino acid 626 of SEQ ID NO: 16 is G, and the amino acid in the capsid protein corresponding to amino acid 718 of SEQ ID NO: 16 is G; (b) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M; (c) the amino acid in the capsid protein corresponding to amino acid 505 of SEQ ID NO: 16 is R, and the amino acid in the capsid protein corresponding to amino acid 687 of SEQ ID NO: 16 is R; (d) the amino acid in the capsid protein corresponding to amino acid 346 of SEQ ID NO: 16 is A, and the amino acid in the capsid protein corresponding to amino acid 505 of SEQ ID NO: 16 is R; or (e) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C.

33. The nucleic acid vector or packaging system of clause 31, wherein the AAV capsid protein comprises the amino acid sequence of amino acids 138-736 of SEQ ID NO: 1, 2, 3, 4, 5, 6, 7, 8, 9, 10, 11, 12, 13, 15, 16, or 17.

34. The nucleic acid vector or packaging system of any preceding clause, wherein the AAV capsid protein comprises an amino acid sequence that is at least 85% identical to the amino acid sequence of amino acids 1-736 of SEQ ID NO: 1, 2, 3, 4, 5, 6, 7, 8, 9, 10, 11, 12, 13, 15, 16, or 17.

35. The nucleic acid vector or packaging system of clause 34, wherein: the amino acid in the capsid protein corresponding to amino acid 2 of SEQ ID NO: 16 is T; the amino acid in the capsid protein corresponding to amino acid 65 of SEQ ID NO: 16 is I; the amino acid in the capsid protein corresponding to amino acid 68 of SEQ ID NO: 16 is V; the amino acid in the capsid protein corresponding to amino acid 77 of SEQ ID NO: 16 is R; the amino acid in the capsid protein corresponding to amino acid 119 of SEQ ID NO: 16 is L; the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G.

36. The nucleic acid vector or packaging system of clause 35, wherein: (a) the amino acid in the capsid protein corresponding to amino acid 2 of SEQ ID NO: 16 is T, and the amino acid in the capsid protein corresponding to amino acid 312 of SEQ ID NO: 16 is Q; (b) the amino acid in the capsid protein corresponding to amino acid 65 of SEQ ID NO: 16 is I, and the amino acid in the capsid protein corresponding to amino acid 626 of SEQ ID NO: 16 is Y; (c) the amino acid in the capsid protein corresponding to amino acid 77 of SEQ ID NO: 16 is R, and the amino acid in the capsid protein corresponding to amino acid 690 of SEQ ID NO: 16 is K; (d) the amino acid in the capsid protein corresponding to amino acid 119 of SEQ ID NO: 16 is L, and the amino acid in the capsid protein corresponding to amino acid 468 of SEQ ID NO: 16 is S; (e) the amino acid in the capsid protein corresponding to amino acid 626 of SEQ ID NO: 16 is G, and the amino acid in the capsid protein corresponding to amino acid 718 of SEQ ID NO: 16 is G; (f) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M; (g) the amino acid in the capsid protein corresponding to amino acid 505 of SEQ ID NO: 16 is R, and the amino acid in the capsid protein corresponding to amino acid 687 of SEQ ID NO: 16 is R; (h) the amino acid in the capsid protein corresponding to amino acid 346 of SEQ ID NO: 16 is A, and the amino acid in the capsid protein corresponding to amino acid 505 of SEQ ID NO: 16 is R; or (i) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C.

37. The nucleic acid vector or packaging system of clause 35, wherein the AAV capsid protein comprises the amino acid sequence of amino acids 1-736 of SEQ ID NO: 1, 2, 3, 4, 5, 6, 7, 8, 9, 10, 11, 12, 13, 15, 16, or 17.

38. The nucleic acid vector or packaging system of any preceding clause, wherein the third nucleotide sequence further comprises a transcriptional regulatory element operably linked to the AAV capsid protein coding sequence.

39. The nucleic acid vector or packaging system of clause 38, wherein the transcriptional regulatory element comprises a promoter selected from the group consisting of a constitutive promoter, an inducible promoter, or a native promoter.

40. The nucleic acid vector or packaging system of clause 39, wherein the promoter is selected from the group consisting of a P40 promoter, a metallothionine (MT) promoter, a mouse mammary tumor virus (MMTV) promoter, a T7 promoter, an ecdysone insect promoter, a tetracycline-repressible promoter, a tetracycline-inducible promoter, an RU486-inducible promoter, and a rapamycin-inducible promoter.

41. The nucleic acid vector or packaging system of any preceding clause, wherein the first nucleic acid vector comprises a nucleotide sequence that is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 73 or 77.

42. The nucleic acid vector or packaging system of any preceding clause, wherein the second nucleotide sequence comprises a sequence that is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 71, 75, 78, 80, 82, 84, 85, 86, 87, or 88.

43. The nucleic acid vector or packaging system of any preceding clause, wherein: the first nucleotide sequence comprises a sequence that is at least 85%, 86%, 87%, 88%, 89%, 90%, 91%, 92%, 93%, 94%, 95%, 96%, 97%, 98%, 99%, or 100% identical to a nucleotide sequence set forth in SEQ ID NO: 50, 51, 52, 53, 54, 55, 56, 57, 58, or 59; the second nucleotide sequence comprises a sequence that is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 71, 75, 78, 80, 82, 84, 85, 86, 87, or 88; and the third nucleotide sequence encodes an amino acid sequence that is at least 85%, 86%, 87%, 88%, 89%, 90%, 91%, 92%, 93%, 94%, 95%, 96%, 97%, 98%, 99%, or 100% identical to the amino acid sequence of amino acids 203-736, 138-736, and/or 1-736 of SEQ ID NO: 1, 2, 3, 4, 5, 6, 7, 8, 9, 10, 11, 12, 13, 15, 16, or 17.

44. The nucleic acid vector or packaging system of clause 43, wherein the first nucleic acid vector comprises, from 5′ to 3′: the first nucleotide sequence; the second nucleotide sequence; and the third nucleotide sequence.

45. The packaging system of any one of clauses 5-44, wherein the helper virus gene is derived from a helper virus selected from the group consisting of adenovirus, herpes virus, poxvirus, cytomegalovirus, and baculovirus.

46. The packaging system of any one of clauses 5-45, wherein the helper virus gene is an RNA gene derived from adenovirus selected from the group consisting of E1, E2, E4, and VA.

47. The packaging system of any one of clauses 5-46, wherein the second nucleic acid vector further comprises a transcriptional regulatory element operably linked to the helper virus gene.

48. The packaging system of clause 47, wherein the transcriptional regulatory element comprises a promoter selected from the group consisting of a constitutive promoter, an inducible promoter, or a native promoter.

49. The packaging system of clause 48, wherein the promoter is selected from the group consisting of an RSV LTR promoter, a CMV immediate early promoter, an SV40 promoter, a dihydrofolate reductase promoter, a cytoplasmic β-actin promoter, a phosphoglycerate kinase (PGK) promoter, a metallothionine (MT) promoter, a mouse mammary tumor virus (MMTV) promoter, a T7 promoter, an ecdysone insect promoter, a tetracycline-repressible promoter, a tetracycline-inducible promoter, an RU486-inducible promoter, and a rapamycin-inducible promoter.

50. The packaging system of any one of clauses 5-49, wherein the second nucleic acid vector comprises a nucleotide sequence that is at least 85%, 86%, 87%, 88%, 89%, 90%, 91%, 92%, 93%, 94%, 95%, 96%, 97%, 98%, 99%, or 100% identical to a nucleotide sequence set forth in SEQ ID NO: 60, 61, or 62.

51. The packaging system of any one of clauses 5-50, wherein the second nucleic acid vector comprises a nucleotide sequence that is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 63.

52. The packaging system of any one of clauses 5-45, wherein the helper virus gene is a gene derived from herpes virus selected from the group consisting of UL5/8/52, ICP0, ICP4, ICP22, and UL30/UL42.

53. The packaging system of clause 52, wherein the second nucleic acid vector further comprises a transcriptional regulatory element operably linked to the helper virus gene.

54. The packaging system of clause 53, wherein the transcriptional regulatory element comprises a promoter selected from the group consisting of a constitutive promoter, an inducible promoter, or a native promoter.

55. The packaging system of clause 54, wherein the promoter is selected from the group consisting of an RSV LTR promoter, a CMV immediate early promoter, an SV40 promoter, a dihydrofolate reductase promoter, a cytoplasmic β-actin promoter, a phosphoglycerate kinase (PGK) promoter, a metallothionine (MT) promoter, a mouse mammary tumor virus (MMTV) promoter, a T7 promoter, an ecdysone insect promoter, a tetracycline-repressible promoter, a tetracycline-inducible promoter, an RU486-inducible promoter, and a rapamycin-inducible promoter.

56. A host cell comprising the nucleic acid vector of any one of clauses 1-4, or 9-44, or the packaging system of any one of clauses 5-55.

57. The host cell of clause 56, wherein the host cell is a mammalian cell.

58. The host cell of clause 57, wherein the mammalian cell is selected from the group consisting of a COS cell, a CHO cell, a BHK cell, an MDCK cell, an HEK293 cell, an HEK293T cell, an HEK293F cell, an NSO cell, a PER.C6 cell, a VERO cell, a CRL7O3O cell, an HsS78Bst cell, a HeLa cell, an NIH 3T3 cell, a HepG2 cell, an SP210 cell, an R1.1 cell, a B-W cell, an L-M cell, a BSC1 cell, a BSC40 cell, a YB/20 cell, and a BMT10 cell.

59. The host cell of clause 57 or 58, wherein the mammalian cell is an HEK293 cell.

60. A method for recombinant preparation of an rAAV, the method comprising introducing the packaging system of any one of clauses 5-55 into a mammalian cell under conditions whereby the rAAV is produced.

61. The method of clause 60, wherein the ratio of the first nucleic acid vector to the second nucleic acid vector or the ratio of the second nucleic acid vector to the first nucleic acid vector is selected from the group consisting of: 1:0.2, 1:0.4, 1:0.6, 1:0.8, 1:1, 1:2, 1:3, or 1:4.

62. The method of clause 60 or 61, wherein the ratio of the first nucleic acid vector to the second nucleic acid vector or the ratio of the second nucleic acid vector to the first nucleic acid vector is 1:2.

63. The method of clause 60 or 61, wherein the ratio of the first nucleic acid vector to the second nucleic acid vector or the ratio of the second nucleic acid vector to the first nucleic acid vector is from 1:0.2 to 1:1.

64. The method of clause 63, wherein the ratio of the first nucleic acid vector to the second nucleic acid vector or the ratio of the second nucleic acid vector to the first nucleic acid vector is 1:0.6.

65. The method of clause 63, wherein the ratio of the first nucleic acid vector to the second nucleic acid vector or the ratio of the second nucleic acid vector to the first nucleic acid vector is 1:0.8.

66. The method of clause 63, wherein the ratio of the first nucleic acid vector to the second nucleic acid vector or the ratio of the second nucleic acid vector to the first nucleic acid vector is 1:1.

67. The method of any one of clauses 60-66, wherein the method comprises introducing from 0.1 to 4 μg DNA/1E6 cells of the packaging system.

68. The method of any one of clauses 60-67, wherein the method comprises introducing from 0.5 to 1 μg DNA/1E6 cells of the packaging system.

69. The method of any one of clauses 60-68, wherein the method comprises introducing 0.6, 0.7, 0.8, 0.9, or 1 μg DNA/1E6 cells of the packaging system.

70. The method of any one of clauses 60-68, wherein the method comprises introducing 0.75 μg DNA/1E6 cells of the packaging system.

71. The method of any one of clauses 60-70, wherein the method results in an increased rAAV titer as compared to a method that comprises producing rAAV using a mammalian cell comprising: (i) a first vector comprising a nucleotide sequence encoding the AAV Rep protein and the AAV capsid protein; (ii) a second vector comprising the rAAV genome; and (iii) a third vector comprising the one or more helper virus genes.

72. The method of any one of clauses 60-70, wherein the method results in an increased percentage of intact vector genomes as compared to a method that comprises producing rAAV using a mammalian cell comprising: (i) a first vector comprising a nucleotide sequence encoding the AAV Rep protein and the AAV capsid protein; (ii) a second vector comprising the rAAV genome; and (iii) a third vector comprising the one or more helper virus genes.

73. The method of any one of clauses 60-72, wherein the mammalian cell is selected from the group consisting of a COS cell, a CHO cell, a BHK cell, an MDCK cell, an HEK293 cell, an HEK293T cell, an HEK293F cell, an NSO cell, a PER.C6 cell, a VERO cell, a CRL7O3O cell, an HsS78Bst cell, a HeLa cell, an NIH 3T3 cell, a HepG2 cell, an SP210 cell, an R1.1 cell, a B-W cell, an L-M cell, a BSC1 cell, a BSC40 cell, a YB/20 cell, and a BMT10 cell.

74. The method of any one of clauses 60-73, wherein the mammalian cell is an HEK293 cell.

75. The method of any one of clauses 60-74, wherein the mammalian cell is incubated in a cell culture.

76. A population of host cells as defined in any one of clauses 56-59, wherein the host cells are provided in a cell culture.

77. The method of clause 75 or the population of host cell of clause 76, wherein the cell culture has a volume of at least 2 liters, at least 50 liters, or at least 2000 liters.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1. A first nucleic acid vector comprising:
a first nucleotide sequence encoding an AAV Rep protein;
a second nucleotide sequence comprising a recombinant AAV (rAAV) genome comprising a transgene; and
a third nucleotide sequence encoding an AAV capsid protein,
wherein the nucleic acid vector does not comprise a helper virus gene.

2. (canceled)

3. The nucleic acid vector of claim 1, comprising from 5′ to 3′:
the first nucleotide sequence encoding an AAV Rep protein;
the second nucleotide sequence comprising a recombinant AAV (rAAV) genome comprising a transgene; and
the third nucleotide sequence encoding an AAV capsid protein.

4. (canceled)

5. A recombinant AAV (rAAV) packaging system, comprising:
(i) a first nucleic acid vector comprising:
a first nucleotide sequence encoding an AAV Rep protein;
a second nucleotide sequence comprising a recombinant AAV (rAAV) genome comprising a transgene; and
a third nucleotide sequence encoding an AAV capsid protein, and
(ii) a second nucleic acid vector comprising a helper virus gene.

6. The packaging system of claim 5, wherein the first nucleic acid vector comprises from 5′ to 3′:
the first nucleotide sequence encoding an AAV Rep protein;
the second nucleotide sequence comprising a recombinant AAV (rAAV) genome comprising a transgene; and
the third nucleotide sequence encoding an AAV capsid protein.

7-15. (canceled)

16. The nucleic acid vector of claim 1, wherein the rAAV genome further comprises a 5′ inverted terminal repeat (5′ ITR) nucleotide sequence 5′ of the transgene, and a 3′ inverted terminal repeat (3′ ITR) nucleotide sequence 3′ of the transgene.

17. The packaging system of claim 5, wherein the rAAV genome further comprises a 5′ inverted terminal repeat (5′ ITR) nucleotide sequence 5′ of the transgene, and a 3′ inverted terminal repeat (3′ ITR) nucleotide sequence 3′ of the transgene.

18. The packaging system of claim 5, wherein the AAV Rep protein is an AAV2 Rep protein or a variant thereof.

19-26. (canceled)

27. The packaging system of claim 5, wherein the helper virus gene is an RNA gene derived from adenovirus selected from the group consisting of E1, E2, E4, and VA.

28-30. (canceled)

31. A recombinant packaging system comprising:
(i) a first nucleic acid vector comprising:
a first nucleotide sequence encoding an AAV Rep protein;
a second nucleotide sequence comprising a recombinant AAV (rAAV) genome comprising a transgene; and
a third nucleotide sequence encoding an AAV capsid protein, and
(ii) a second nucleic acid vector comprising a nucleotide sequence that is at least 85% identical to a nucleotide sequence set forth in SEQ ID NO: 60, 61, 62, or 63.

32. (canceled)

33. The packaging system of claim 5, wherein the helper virus gene is a gene derived from herpes virus selected from the group consisting of UL5/8/52, ICPO, ICP4, ICP22, and UL30/UL42.

34-36. (canceled)

37. A host cell comprising:
(i) a first nucleic acid vector comprising:
a first nucleotide sequence encoding an AAV Rep protein;
a second nucleotide sequence comprising a recombinant AAV (rAAV) genome comprising a transgene; and
a third nucleotide sequence encoding an AAV capsid protein, and
(ii) a second nucleic acid vector comprising a helper virus gene.

38. The host cell of claim 37, wherein the host cell is a mammalian cell.

39. The host cell of claim 38, wherein the mammalian cell is selected from the group consisting of a COS cell, a CHO cell, a BHK cell, an MDCK cell, an HEK293 cell, an HEK293T cell, an HEK293F cell, an NSO cell, a PER.C6 cell, a VERO cell, a CRL7O3O cell, an HsS78Bst cell, a HeLa cell, an NIH 3T3 cell, a HepG2 cell, an SP210 cell, an R1.1 cell, a B-W cell, an L-M cell, a BSC1 cell, a BSC40 cell, a YB/20 cell, and a BMT10 cell.

40. The host cell of claim 38 or 39, wherein the mammalian cell is an HEK293 cell.

41. A method for recombinant preparation of an rAAV, the method comprising introducing the packaging system comprising:
(i) a first nucleic acid vector comprising:
a first nucleotide sequence encoding an AAV Rep protein;
a second nucleotide sequence comprising a recombinant AAV (rAAV) genome comprising a transgene; and
a third nucleotide sequence encoding an AAV capsid protein, and
(ii) a second nucleic acid vector comprising a helper virus gene into a mammalian cell under conditions whereby the rAAV is produced.

42. A method for recombinant preparation of an rAAV, the method comprising introducing the packaging system of claim 5 into a mammalian cell under conditions whereby the rAAV is produced, wherein the ratio of the first nucleic acid vector to the second nucleic acid vector or the ratio of the second nucleic acid vector to the first nucleic acid vector is selected from the group consisting of: 1:0.2, 1:0.4, 1:0.6, 1:0.8, 1:1, 1:2, 1:3, or 1:4.

43-50. (canceled)

51. A method of comprising introducing the packaging system of claim 5 into a mammalian cell under conditions whereby the rAAV is produced, wherein the method comprises introducing 0.75 μg DNA/1E6 cells of the packaging system.

52. The A method comprising introducing the packaging system of claim 5 into a mammalian cell under conditions whereby the rAAV is produced, wherein the method results in an increased rAAV titer as compared to a method that comprises producing rAAV using a mammalian cell comprising:
(i) a first vector comprising a nucleotide sequence encoding the AAV Rep protein and the AAV capsid protein;
(ii) a second vector comprising the rAAV genome; and
(iii) a third vector comprising the one or more helper virus genes.

53. The A method comprising introducing the packaging system of claim 5 into a mammalian cell under conditions whereby the rAAV is produced, wherein the method results in an increased percentage of intact vector genomes as compared to a method that comprises producing rAAV using a mammalian cell comprising:
(i) a first vector comprising a nucleotide sequence encoding the AAV Rep protein and the AAV capsid protein;
(ii) a second vector comprising the rAAV genome; and
(iii) a third vector comprising the one or more helper virus genes.

54-56. (canceled)

57. A population of the host cells of claim 37, wherein the population of host cells is provided in a cell culture.

58. The method of claim 41, wherein the cell culture has a volume of at least 2000 liters.


SEQUENCE LISTING (106 sequences)

SEQ ID NO: 1 - protein, 736 aa, Artificial Sequence
  Adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 2 - protein, 736 aa, Artificial Sequence
  AAV isolate
MTADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKQLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 3 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTGGNFHPSPLMGGFGMKHPPPQILIKNTPVPADPPTAFNKDKLNSFITQYSTGQVSVEIEWELQKENSKRWNPEIQYTSNYYKSNNVEFAVNTGGVYSEPRPIGTRYLTRNL

SEQ ID NO: 4 - protein, 736 aa, Artificial Sequence
  AAV isolate
MAADGYLPDWLEDNLSEGIREWWALKPGAPQPKANQQHQDNARGLVLPGYKYLGPGNGLDKGEPINAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTYGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 5 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIDKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 6 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVLQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGSSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 7 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQARAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 8 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAVDAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 9 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPREPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 10 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASCGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 11 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDRAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKKNSKRWNPEIQYTSNYYKSNNVEFAVNTEGVYSEPRPIGTRYLTRNL

SEQ ID NO: 12 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPHDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFNVAGPSNMAVQGRNYIPGPSYRQQRVSTTVTQNNNSEFAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSMEIEWELQKENSKRWNPEIQYTSNYYKSNNVEFAVNTEGVYSEPRPIGTRYLTRNL

SEQ ID NO: 13 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 14 - skipped

SEQ ID NO: 15 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWERQKENSKRWNPEIQYTSNYYKSNNVEFAVNTEGVYSEPRPIGTRYLTRNL

SEQ ID NO: 16 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFADSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 17 - protein, 736 aa, Artificial Sequence
  AAV isolate
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEIAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYCKSNNVEFAVNTEGVYSEPRPIGTRYLTRNL

SEQ ID NO: 18 - DNA, 365 bp, Artificial Sequence
  Synthetic polynucleotide
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatg

SEQ ID NO: 19 - DNA, 380 bp, Artificial Sequence
  Synthetic polynucleotide
ggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatg

SEQ ID NO: 20 - DNA, 479 bp, Artificial Sequence
  Synthetic polynucleotide
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggag

SEQ ID NO: 21 - DNA, 1246 bp, Artificial Sequence
  Synthetic polynucleotide
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcgg

SEQ ID NO: 22 - DNA, 953 bp, Artificial Sequence
  Synthetic polynucleotide
aattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaag

SEQ ID NO: 23 - DNA, 1168 bp, Artificial Sequence
  Synthetic polynucleotide
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaggatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaggggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 24 - DNA, 92 bp, Artificial Sequence
  Synthetic polynucleotide
aagaggtaagggtttaagggatggttggttggtggggtattaatgtttaattacctggagcacctgcctgaaatcactttttttcaggttgg

SEQ ID NO: 25 - DNA, 79 bp, Artificial Sequence
  Synthetic polynucleotide
aagaggtaagggtttaagggatggttggttggtggggtattaatgtttaattacctggagcacctgcctgaaatcactt

SEQ ID NO: 26 - DNA, 95 bp, Artificial Sequence
  Synthetic polynucleotide
cctctgctaaccatgttcatgccttcttctttttcctacagctcctgggcaacgtgctggttattgtgctgtctcatcattttggcaaagaattc

SEQ ID NO: 27 - DNA, 90 bp, Artificial Sequence
  Synthetic polynucleotide
cctctgctaaccatgttcatgccttcttctttttcctacagctcctgggcaacgtgctggttattgtgctgtctcatcattttggcaaag

SEQ ID NO: 28 - DNA, 918 bp, Artificial Sequence
  Synthetic polynucleotide
ggcatcctaaaaaatattcagtggaaacgtaaaaacattaaagactgattaaacatcgcagcatgacacagatttagcaactgagcataaataatttgactcggatactgctccaaaatccgaagaggaccaatttcttccaggaggacaactacctcgtcctctgcagacccctctcctcggcagctgaaggagtgtggccaatctgcctccacctccccgcggaccccctactctcaggacctcctgcagcaccccaaactggaagtggccgctgcagacccaaggacgaggggcacgcgggagccggcagccctagtggagcggttggagatgttgaggtgggagggtcacccaggtggggtgaggctggggtaggtagcggagtgaacggcttccgaagctctgggccgcccccaggttggactaagcaggcgctctgtcttcgcccccgcccagggtgggcgtctcctgaggactccccgccacacctgacccgagaccgcgcgcccagcctagaacgcttccccgacccagcgtagggccgccgcgactggcgggcgagggtcggcgggaggcctggcgaacccgggggcgggaccaggcgggcaaggcccggctgccgcagcgccgctctgcgcgaggcggctccgccgcggcggagggatacggcgcaccatatatatatcgcggggcgcagactcgcgctccggcagtggtgctgggagtgtcgtggacgccgtgccgttactcgtagtcaggcggcggcgcaggcggcggcggcggcatagcgcacagcgcgccttagcagcagcagcagcagcagcggcatcggaggtacccccgccgtcgcagcccccgcgctggtgcagccaccctcgctccctctgctcttcctcccttcgctcgcaccaagag

SEQ ID NO: 29 - DNA, 192 bp, Artificial Sequence
  Synthetic polynucleotide
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 30 - DNA, 205 bp, Artificial Sequence
  Synthetic polynucleotide
aatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagg

SEQ ID NO: 31 - DNA, 913 bp, Artificial Sequence
  Synthetic polynucleotide
tagggaggtcctgcacagaaggggaggagggggcagcagctgtctgaccactgttggtcttgcaacttgtgtccccaggttaatttttaaaaagcagtcaaaagtccaagtggcccttggcagcatttactctctctgtttgctctggttaataatctcaggagcacaaacattcctggaggcaggagaagaaatcaacatcctggacttatcctctgggcctctccccacccccaggattgtaactgaaatgcttcactggtgctccttttgttttaaggcattggatcttcatagctactgatcgtgcccaagcacacagtatctgcagcaaccacttaggcctccaggaatgtggtgaccattgaccctaattcattccccttcatggatcctatgtaaccatcctccaaaaagagctttcgcaaactcaaataaacacaggaaaggaagaccttcttatctttgagagtatatgtttagccctatagctctaacccactctgatctcccagggcggcagtaagtcttcagcatcaggcattttggggtgactcagtaaatggtagatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggac

SEQ ID NO: 32 - DNA, 423 bp, Artificial Sequence
  Synthetic polynucleotide
gctctaacccactctgatctcccagggcggcagtaagtcttcagcatcaggcattttggggtgactcagtaaatggtagatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggac

SEQ ID NO: 33 - DNA, 592 bp, Artificial Sequence
  Synthetic polynucleotide
gtaaattttatggaatgtgaatcataattcaatttttcaacatgcgttaggagggacatttcaaactcttttttaccctagactttcctaccatcacccagagtatccagccaggaggggaggggctagagacaccagaagtttagcagggaggagggcgtagggattcggggaatgaagggatgggattcagactagggccaggacccagggatggagagaaagagatgagagtggtttgggggcttggtgacttagagaacagagctgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 34 - DNA, 72 bp, Artificial Sequence
  Synthetic polynucleotide
gggggaggctgctggtgaatattaaccaaggtcaccccagttatcggaggagcaaacaggggctaagtccac

SEQ ID NO: 35 - DNA, 170 bp, Artificial Sequence
  Synthetic polynucleotide
cgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatca

SEQ ID NO: 36 - DNA, 1873 bp, Artificial Sequence
  Synthetic polynucleotide
gatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaatt

SEQ ID NO: 37 - DNA, 1061 bp, Artificial Sequence
  Synthetic polynucleotide
tagggaggtcctgcacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcgggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactaaaacaggtaagtccggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgatgcctctactaaccatgttcatgttttctttttttttctacaggtcctgggtgacgaacag

SEQ ID NO: 38 - DNA, 398 bp, Artificial Sequence
  Synthetic polynucleotide
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagg

SEQ ID NO: 39 - DNA, 145 bp, Artificial Sequence
  Synthetic polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 40 - DNA, 145 bp, Artificial Sequence
  Synthetic polynucleotide
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 41 - DNA, 106 bp, Artificial Sequence
  Synthetic polynucleotide
ctgcgcgctcgctcgctcactgaggccgcccgggcaaagcccgggcgtcgggcgacctttggtcgcccggcctcagtgagcgagcgagcgcgcagagagggagtgg

SEQ ID NO: 42 - DNA, 167 bp, Artificial Sequence
  Synthetic polynucleotide
ctctcccccctgtcgcgttcgctcgctcgctggctcgtttgggggggtggcagctcaaagagctgccagacgacggccctctggccgtcgcccccccaaacgagccagcgagcgagcgaacgcgacaggggggagagtgccacactctcaagcaagggggttttgta

SEQ ID NO: 43 - DNA, 167 bp, Artificial Sequence
  Synthetic polynucleotide
tacaaaacctccttgcttgagagtgtggcactctcccccctgtcgcgttcgctcgctcgctggctcgtttgggggggtggcagctcaaagagctgccagacgacggccctctggccgtcgcccccccaaacgagccagcgagcgagcgaacgcgacaggggggagag

SEQ ID NO: 44 - DNA, 143 bp, Artificial Sequence
  Synthetic polynucleotide
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 45 - DNA, 46 bp, Artificial Sequence
  Synthetic polynucleotide
ggaggggtggagtcgtgacgtgaattacgtcatagggttagggagg

SEQ ID NO: 46 - DNA, 37 bp, Artificial Sequence
  Synthetic polynucleotide
gtagataagtagcatggcgggttaatcattaactaca

SEQ ID NO: 47 - DNA, 96 bp, Artificial Sequence
  Synthetic polynucleotide
tcctgtattagaggtcacgtgagtgttttgcgacattttgcgacaccatgtggtcacgctgggtatttaagcccgagtgagcacgcagggtctcca

SEQ ID NO: 48 - DNA, 135 bp, Artificial Sequence
  Synthetic polynucleotide
gtcacaaagaccagaaatggcgccggaggcgggaacaaggtggtggatgagtgctacatccccaattacttgctccccaaaacccagcctgagctccagtgggcgtggactaatatggaacagtatttaagcgcc

SEQ ID NO: 49 - DNA, 154 bp, Artificial Sequence
  Synthetic polynucleotide
ggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtca

SEQ ID NO: 50 - DNA, 1866 bp, Artificial Sequence
  Synthetic polynucleotide
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 51 - DNA, 1995 bp, Artificial Sequence
  Synthetic polynucleotide
tcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 52 - DNA, 1930 bp, Artificial Sequence
  Synthetic polynucleotide
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgacttaaaccaggtatggctgccgatggttatcttccagattggctcgaggacaaccttagt

SEQ ID NO: 53 - DNA, 2059 bp, Artificial Sequence
  Synthetic polynucleotide
tcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgacttaaaccaggtatggctgccgatggttatcttccagattggctcgaggacaaccttagt

SEQ ID NO: 54 - DNA, 1258 bp, Artificial Sequence
  Synthetic polynucleotide
atggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgacttaaaccaggtatggctgccgatggttatcttccagattggctcgaggacaaccttagt

SEQ ID NO: 55 - DNA, 1531 bp, Artificial Sequence
  Synthetic polynucleotide
gtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgacttaaaccaggtatggctgccgatggttatcttccagattggctcgaggacaaccttagt

SEQ ID NO: 56 - DNA, 1194 bp, Artificial Sequence
  Synthetic polynucleotide
atggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 57 - DNA, 1467 bp, Artificial Sequence
  Synthetic polynucleotide
gtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 58 - DNA, 1930 bp, Artificial Sequence; identical to SEQ ID NO: 52
  Synthetic polynucleotide

SEQ ID NO: 59 - DNA, 2059 bp, Artificial Sequence; identical to SEQ ID NO: 53
  Synthetic polynucleotide

SEQ ID NO: 60 - DNA, 5336 bp, Artificial Sequence
  Synthetic polynucleotide
ggtacccaactccatgcttaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactaggagacactttcaataaaggcaaatgtttttatttgtacactctcgggtgattatttaccccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcggggaggcggcggcgacggcgacggggacgacacgtcctccatggttggtggacgtcgcgccgcaccgcgtccgcgctcgggggtggtttcgcgctgctcctcttcccgactggccatttccttctcctataggcagaaaaagatcatggagtcagtcgagaaggaggacagcctaaccgccccctttgagttcgccaccaccgcctccaccgatgccgccaacgcgcctaccaccttccccgtcgaggcacccccgcttgaggaggaggaagtgattatcgagcaggacccaggttttgtaagcgaagacgacgaggatcgctcagtaccaacagaggataaaaagcaagaccaggacgacgcagaggcaaacgaggaacaagtcgggcggggggaccaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctgttctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcgacgaagtgccaaaaatctttgagggtcttggacgcgacgagaaacgcgcggcaaacgctctgcaacaagaaaacagcgaaaatgaaagtcactgtggagtgctggtggaacttgagggtgacaacgcgcgcctagccgtgctgaaacgcagcatcgaggtcacccactttgcctacccggcacttaacctaccccccaaggttatgagcacagtcatgagcgagctgatcgtgcgccgtgcacgacccctggagagggatgcaaacttgcaagaacaaaccgaggagggcctacccgcagttggcgatgagcagctggcgcgctggcttgagacgcgcgagcctgccgacttggaggagcgacgcaagctaatgatggccgcagtgcttgttaccgtggagcttgagtgcatgcagcggttctttgctgacccggagatgcagcgcaagctagaggaaacgttgcactacacctttcgccagggctacgtgcgccaggcctgcaaaatttccaacgtggagctctgcaacctggtctcctaccttggaattttgcacgaaaaccgcctcgggcaaaacgtgcttcattccacgctcaagggcgaggcgcgccgcgactacgtccgcgactgcgtttacttatttctgtgctacacctggcaaacggccatgggcgtgtggcagcaatgcctggaggagcgcaacctaaaggagctgcagaagctgctaaagcaaaacttgaaggacctatggacggccttcaacgagcgctccgtggccgcgcacctggcggacattatcttccccgaacgcctgcttaaaaccctgcaacagggtctgccagacttcaccagtcaaagcatgttgcaaaactttaggaactttatcctagagcgttcaggaattctgcccgccacctgctgtgcgcttcctagcgactttgtgcccattaagtaccgtgaatgccctccgccgctttggggtcactgctaccttctgcagctagccaactaccttgcctaccactccgacatcatggaagacgtgagcggtgacggcctactggagtgtcactgtcgctgcaacctatgcaccccgcaccgctccctggtctgcaattcgcaactgcttagcgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacatccttggccaattgcaagccatcaacaaagcccgccaagagtttctgctacgaaagggacggggggtttacctggacccccagtccggcgaggagctcaacccaatccccccgccgccgcagccctatcagcagccgcgggcccttgcttcccaggatggcacccaaaaagaagctgcagctgccgccgccgccacccacggacgaggaggaatactgggacagtcaggcagaggaggttttggacgaggaggaggagatgatggaagactgggacagcctagacgaagcttccgaggccgaagaggtgtcagacgaaacaccgtcaccctcggtcgcattcccctcgccggcgccccagaaattggcaaccgttcccagcatcgctacaacctccgctcctcaggcgccgccggcactgcctgttcgccgacccaaccgtagatgggacaccactggaaccagggccggtaagtctaagcagccgccgccgttagcccaagagcaacaacagcgccaaggctaccgctcgtggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccctactgcaccggcggcagcggcagcggcagcaacagcagcggtcacacagaagcaaaggcgaccggatagcaagactctgacaaagcccaagaaatccacagcggcggcagcagcaggaggaggagcgctgcgtctggcgcccaacgaacccgtatcgacccgcgagcttagaaataggatttttcccactctgtatgctatatttcaacaaagcaggggccaagaacaagagctgaaaataaaaaacaggtctctgcgctccctcacccgcagctgcctgtatcacaaaagcgaagatcagcttcggcgcacgctggaagacgcggaggctctcttcagcaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcctcgaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcg

SEQ ID NO: 61 - DNA, 3201 bp, Artificial Sequence
  Synthetic polynucleotide
cccgggcgttttagggcggagtaacttgcatgtattgggaattgtagtttttttaaaatgggaagtgacgtatcgtgggaaaacggaagtgaagatttgaggaagttgtgggttttttggctttcgtttctgggcgtaggttcgcgtgcggttttctgggtgttttttgtggactttaaccgttacgtcattttttagtcctatatatactcgctctgtacttggccctttttacactgtgactgattgagctggtgccgtgtcgagtggtgttttttaataggtttttttactggtaaggctgactgttatggctgccgctgtggaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgttaaccaacctgatgtgtttaccgagtcttacattatgactccggacatgaccgaggaactgtcggtggtgctttttaatcacggtgaccagtttttttacggtcacgccggcatggccgtagtccgtcttatgcttataagggttgtttttcctgttgtaagacaggcttctaatgtttaaatgtttttttttttgttattttattttgtgtttaatgcaggaacccgcagacatgtttgagagaaaaatggtgtctttttctgtggtggttccggaacttacctgcctttatctgcatgagcatgactacgatgtgcttgcttttttgcgcgaggctttgcctgattttttgagcagcaccttgcattttatatcgccgcccatgcaacaagcttacataggggctacgctggttagcatagctccgagtatgcgtgtcataatcagtgtgggttcttttgtcatggttcctggcggggaagtggccgcgctggtccgtgcagacctgcacgattatgttcagctggccctgcgaagggacctacgggatcgcggtatttttgttaatgttccgcttttgaatcttatacaggtctgtgaggaacctgaatttttgcaatcatgattcgctgcttgaggctgaaggtggagggcgctctggagcagatttttacaatggccggacttaatattcgggatttgcttagagacatattgataaggtggcgagatgaaaattatttgggcatggttgaaggtgctggaatgtttatagaggagattcaccctgaagggtttagcctttacgtccacttggacgtgagggcagtttgccttttggaagccattgtgcaacatcttacaaatgccattatctgttctttggctgtagagtttgaccacgccaccggaggggagcgcgttcacttaatagatcttcattttgaggttttggataatcttttggaataaaaaaaaaaaaacatggttcttccagctcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgagctaagcgacgagaccggagacgcagatctgtttgtcacgcccgcacctggttttgcttcaggaaatatgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttccccttcgccgcccgttgagcaaccgcaagttggacagcagcctgtggctcagcagctggacagcgacatgaacttaagcgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatcaatataagctatgtggtggtggggctatactactgaatgaaaaatgacttgaaattttctgcaattgaaaaataaacacgttgaaacataacatgcaacaggttcacgattctttattcctgggcaatgtaggagaaggtgtaagagttggtagcaaaagtttcagtggtgtattttccactttcccaggaccatgtaaaagacatagagtaagtgcttacctcgctagtttctgtggattcactagaa

SEQ ID NO: 62 - DNA, 743 bp, Artificial Sequence
  Synthetic polynucleotide
tcgatgtaggatgttgcccctcctgacgcggtaggagaaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggcccgagactcgaaccgggggtcctgcgactcaacccttggaaaataaccctccggctacagggagcgagccacttaatgctttcgctttccagcctaaccgcttacgccgcgcgcggccagtggccaaaaaagctagcgcagcagccgccgcgcctggaaggaagccaaaaggagcgctcccccgttgtctgacgtcgcacacctgggttcgacacgcgggcggtaaccgcatggatcacggcggacggccggatccggggttcgaaccccggtcgtccgccatgatacccttgcgaatttatccaccagaccacggaagagtgcccgcttacaggctctccttttgcacggtctagagcgtcaacgactgcgcacgcctcaccggccagagcgtcccgaccatggagcactttttgccgctgcgcaacatctggaaccgcgtccgcgactttccgcgcgcctccaccaccgccgccggcatcacctggatgtccaggtacatctacggattacg

SEQ ID NO: 63 - DNA, 9280 bp, Artificial Sequence
  Synthetic polynucleotide
ggtacccaactccatgcttaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactaggagacactttcaataaaggcaaatgtttttatttgtacactctcgggtgattatttaccccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcggggaggcggcggcgacggcgacggggacgacacgtcctccatggttggtggacgtcgcgccgcaccgcgtccgcgctcgggggtggtttcgcgctgctcctcttcccgactggccatttccttctcctataggcagaaaaagatcatggagtcagtcgagaaggaggacagcctaaccgccccctttgagttcgccaccaccgcctccaccgatgccgccaacgcgcctaccaccttccccgtcgaggcacccccgcttgaggaggaggaagtgattatcgagcaggacccaggttttgtaagcgaagacgacgaggatcgctcagtaccaacagaggataaaaagcaagaccaggacgacgcagaggcaaacgaggaacaagtcgggcggggggaccaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctgttctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcgacgaagtgccaaaaatctttgagggtcttggacgcgacgagaaacgcgcggcaaacgctctgcaacaagaaaacagcgaaaatgaaagtcactgtggagtgctggtggaacttgagggtgacaacgcgcgcctagccgtgctgaaacgcagcatcgaggtcacccactttgcctacccggcacttaacctaccccccaaggttatgagcacagtcatgagcgagctgatcgtgcgccgtgcacgacccctggagagggatgcaaacttgcaagaacaaaccgaggagggcctacccgcagttggcgatgagcagctggcgcgctggcttgagacgcgcgagcctgccgacttggaggagcgacgcaagctaatgatggccgcagtgcttgttaccgtggagcttgagtgcatgcagcggttctttgctgacccggagatgcagcgcaagctagaggaaacgttgcactacacctttcgccagggctacgtgcgccaggcctgcaaaatttccaacgtggagctctgcaacctggtctcctaccttggaattttgcacgaaaaccgcctcgggcaaaacgtgcttcattccacgctcaagggcgaggcgcgccgcgactacgtccgcgactgcgtttacttatttctgtgctacacctggcaaacggccatgggcgtgtggcagcaatgcctggaggagcgcaacctaaaggagctgcagaagctgctaaagcaaaacttgaaggacctatggacggccttcaacgagcgctccgtggccgcgcacctggcggacattatcttccccgaacgcctgcttaaaaccctgcaacagggtctgccagacttcaccagtcaaagcatgttgcaaaactttaggaactttatcctagagcgttcaggaattctgcccgccacctgctgtgcgcttcctagcgactttgtgcccattaagtaccgtgaatgccctccgccgctttggggtcactgctaccttctgcagctagccaactaccttgcctaccactccgacatcatggaagacgtgagcggtgacggcctactggagtgtcactgtcgctgcaacctatgcaccccgcaccgctccctggtctgcaattcgcaactgcttagcgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacatccttggccaattgcaagccatcaacaaagcccgccaagagtttctgctacgaaagggacggggggtttacctggacccccagtccggcgaggagctcaacccaatccccccgccgccgcagccctatcagcagccgcgggcccttgcttcccaggatggcacccaaaaagaagctgcagctgccgccgccgccacccacggacgaggaggaatactgggacagtcaggcagaggaggttttggacgaggaggaggagatgatggaagactgggacagcctagacgaagcttccgaggccgaagaggtgtcagacgaaacaccgtcaccctcggtcgcattcccctcgccggcgccccagaaattggcaaccgttcccagcatcgctacaacctccgctcctcaggcgccgccggcactgcctgttcgccgacccaaccgtagatgggacaccactggaaccagggccggtaagtctaagcagccgccgccgttagcccaagagcaacaacagcgccaaggctaccgctcgtggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccctactgcaccggcggcagcggcagcggcagcaacagcagcggtcacacagaagcaaaggcgaccggatagcaagactctgacaaagcccaagaaatccacagcggcggcagcagcaggaggaggagcgctgcgtctggcgcccaacgaacccgtatcgacccgcgagcttagaaataggatttttcccactctgtatgctatatttcaacaaagcaggggccaagaacaagagctgaaaataaaaaacaggtctctgcgctccctcacccgcagctgcctgtatcacaaaagcgaagatcagcttcggcgcacgctggaagacgcggaggctctcttcagcaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcctcgaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcgttttagggcggagtaacttgcatgtattgggaattgtagtttttttaaaatgggaagtgacgtatcgtgggaaaacggaagtgaagatttgaggaagttgtgggttttttggctttcgtttctgggcgtaggttcgcgtgcggttttctgggtgttttttgtggactttaaccgttacgtcattttttagtcctatatatactcgctctgtacttggccctttttacactgtgactgattgagctggtgccgtgtcgagtggtgttttttaataggtttttttactggtaaggctgactgttatggctgccgctgtggaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgttaaccaacctgatgtgtttaccgagtcttacattatgactccggacatgaccgaggaactgtcggtggtgctttttaatcacggtgaccagtttttttacggtcacgccggcatggccgtagtccgtcttatgcttataagggttgtttttcctgttgtaagacaggcttctaatgtttaaatgtttttttttttgttattttattttgtgtttaatgcaggaacccgcagacatgtttgagagaaaaatggtgtctttttctgtggtggttccggaacttacctgcctttatctgcatgagcatgactacgatgtgcttgcttttttgcgcgaggctttgcctgattttttgagcagcaccttgcattttatatcgccgcccatgcaacaagcttacataggggctacgctggttagcatagctccgagtatgcgtgtcataatcagtgtgggttcttttgtcatggttcctggcggggaagtggccgcgctggtccgtgcagacctgcacgattatgttcagctggccctgcgaagggacctacgggatcgcggtatttttgttaatgttccgcttttgaatcttatacaggtctgtgaggaacctgaatttttgcaatcatgattcgctgcttgaggctgaaggtggagggcgctctggagcagatttttacaatggccggacttaatattcgggatttgcttagagacatattgataaggtggcgagatgaaaattatttgggcatggttgaaggtgctggaatgtttatagaggagattcaccctgaagggtttagcctttacgtccacttggacgtgagggcagtttgccttttggaagccattgtgcaacatcttacaaatgccattatctgttctttggctgtagagtttgaccacgccaccggaggggagcgcgttcacttaatagatcttcattttgaggttttggataatcttttggaataaaaaaaaaaaaacatggttcttccagctcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgagctaagcgacgagaccggagacgcagatctgtttgtcacgcccgcacctggttttgcttcaggaaatatgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttccccttcgccgcccgttgagcaaccgcaagttggacagcagcctgtggctcagcagctggacagcgacatgaacttaagcgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatcaatataagctatgtggtggtggggctatactactgaatgaaaaatgacttgaaattttctgcaattgaaaaataaacacgttgaaacataacatgcaacaggttcacgattctttattcctgggcaatgtaggagaaggtgtaagagttggtagcaaaagtttcagtggtgtattttccactttcccaggaccatgtaaaagacatagagtaagtgcttacctcgctagtttctgtggattcactagaatcgatgtaggatgttgcccctcctgacgcggtaggagaaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggcccgagactcgaaccgggggtcctgcgactcaacccttggaaaataaccctccggctacagggagcgagccacttaatgctttcgctttccagcctaaccgcttacgccgcgcgcggccagtggccaaaaaagctagcgcagcagccgccgcgcctggaaggaagccaaaaggagcgctcccccgttgtctgacgtcgcacacctgggttcgacacgcgggcggtaaccgcatggatcacggcggacggccggatccggggttcgaaccccggtcgtccgccatgatacccttgcgaatttatccaccagaccacggaagagtgcccgcttacaggctctccttttgcacggtctagagcgtcaacgactgcgcacgcctcaccggccagagcgtcccgaccatggagcactttttgccgctgcgcaacatctggaaccgcgtccgcgactttccgcgcgcctccaccaccgccgccggcatcacctggatgtccaggtacatctacggattacg

SEQ ID NO: 64 - protein, 621 aa, Artificial Sequence
  AAV2 Rep
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 65 - DNA, 198 bp, Artificial Sequence
  Synthetic polynucleotide
gatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 66 - DNA, 4186 bp, Artificial Sequence
  Synthetic polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcatatgcggccgcagcattagcttccatttatgcagtgtaaatggtgagaacagccccgactgaatacccagagcatcatctcgtctgtgtcattcatgcacataacatatctcagcgaggtggcccttctgtcctctttgcagagacccagccaccatactagtacctagagaactggctggatttcagccccgatacctccgggcttttgctcatgttcgcctcatagggtcatctgggtggttgcctaaggaaaagtatgtcatggagactaacttgcttggcattgaataaaaggtgagttgagagtggagcgtgtttaaattgcaatcctgcctctatttctgtgcttgcagggaacagtcatccttaattgctatcctccatcatcatcatgattatttctggtttttctctggttgcggagaatccatactccaggtattccaatgtctcagcattgccaggcctgtctgagcgtcaggatgtaggtagtctgggctctctgccttctattcttgtccaggatactctgccaaaagaatcatgttgtggctgccacccctcccacaaagcctcccgcttgggtcagtccaggactggagttgggtatggactgttcatgtctatccactgctacgtcagggcaacacccactgagagtgaccttgtagactgcagtgggagacacccttcaaaacctctcctctcctgtcctgagagccaggttaaaaccatcagccccgcatcctgagtgcaaacttttcctaaccctgctgctaagctagacacctcacttactgagagccagcccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcctctaaggtaaatataaaatttttaagtgtataatgtgttaaactactgattctaattgtttctctcttttagattccaacctttggaactgaccgccaccatgtccaccgctgtgctggagaaccctgggctggggaggaaactgtcagacttcgggcaggagacttcatacattgaggataactgtaaccagaatggcgccatctctctgatcttcagcctgaaggaggaagtgggcgccctggcaaaggtgctgcgcctgtttgaggagaacgacgtgaatctgacccacatcgagtcccggccttctagactgaagaaggacgagtacgagttctttacccacctggataagcggtccctgccagccctgacaaacatcatcaagatcctgaggcacgacatcggagcaaccgtgcacgagctgtctcgggacaagaagaaggataccgtgccctggttccctcggacaatccaggagctggatagatttgccaaccagatcctgtcttacggagcagagctggacgcagatcaccctggcttcaaggacccagtgtatcgggcccggagaaagcagtttgccgatatcgcctacaattataggcacggacagccaatccctcgcgtggagtatatggaggaggagaagaagacctggggcacagtgttcaagaccctgaagagcctgtacaagacacacgcctgctacgagtataaccacatcttccccctgctggagaagtattgtggctttcacgaggacaatatccctcagctggaggacgtgagccagttcctgcagacctgcacaggctttaggctgaggccagtggcaggactgctgagctcccgggacttcctgggaggactggccttcagagtgtttcactgcacccagtacatcaggcacggctccaagccaatgtatacaccagagcccgacatctgtcacgagctgctgggccacgtgcccctgtttagcgatagatccttcgcccagttttcccaggagatcggactggcatctctgggagcacctgacgagtacatcgagaagctggccaccatctattggttcacagtggagtttggcctgtgcaagcagggcgatagcatcaaggcctacggagcaggactgctgtctagcttcggcgagctgcagtattgtctgtccgagaagccaaagctgctgcccctggagctggagaagaccgccatccagaactacaccgtgacagagttccagcccctgtactatgtggccgagtcttttaacgatgccaaggagaaggtgagaaatttcgccgccacaatccctaggcccttcagtgtgcgttacgacccttatacccagaggatcgaggtgctggataatacacagcagctgaagatcctggctgactcaatcaatagcgaaatcggaatcctgtgctccgccctgcagaaaatcaaatgagaattcaaggcctctcgagcctctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaagctttacgtacgatcgtcgagcagctgttgtcctggagaacggagtcctgagcagaaaactctcagactttgggcaggtaagcctgttgggcttccactgctaggagagaattggttccccacatgtgaaagcagtctgggaaatgctggtatttccagtctcctaaggctactaagaaatatgactttatttagaggcgaggaaaatgcccaggaagtcaactgatgagactagtcttaacaagttgaggatacagaaagttggggatctgagctgctaccaacatctgtgtgtctttgggtggctcattggtatcctctgcctattggctttatcttctgtacactgaaaggaaatggctggtccttagtcacctggggtgggagtccctatctctccagggatacttattcaatcctttcttctgggtatcaaaatgacaagcttgtaagaaactgtcctctttcggctttcaggaggtgatgtcgcatgaagagaatttggggggggggacttactcagaaccaaggagggagaaattaaacagagagggaaatgaacaggagttagcccggagcctgaagcaccttggggattatgctgggggtggagggaatccattgtcctccctagggagggcttgcagaacatgttcttttctgtgatatttgtactttccccagattgcaaatcatggtttgtacactgagattcagtctctggaggtaatatgccttttctagcttttccttggacaggactaaggggttgagggttgcctggagtcagagaaatttgtgttaaagaaggttgatatgaaacctgcaggtctagatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 67 - DNA, 171 bp, Artificial Sequence
  Synthetic polynucleotide
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagg

SEQ ID NO: 68 - DNA, 122 bp, Artificial Sequence
  Synthetic polynucleotide
aacttgtttattgcagcttataatggttacaaataaagcaatagcatcacaaatttcacaaataaagcatttttttcactgcattctagttgtggtttgtccaaactcatcaatgtatctta

SEQ ID NO: 69 - DNA, 133 bp, Artificial Sequence
  Synthetic polynucleotide
tgctttatttgtgaaatttgtgatgctattgctttatttgtaaccattataagctgcaataaacaagttaacaacaacaattgcattcattttatgtttcaggttcagggggaggtgtgggaggttttttaaa

SEQ ID NO: 70 - DNA, 3637 bp, Artificial Sequence
  Synthetic polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcatatgcggccgcagcattagcttccatttatgcagtgtaaatggtgagaacagccccgactgaatacccagagcatcatctcgtctgtgtcattcatgcacataacatatctcagcgaggtggcccttctgtcctctttgcagagacccagccaccatactagtacctagagaactggctggatttcagccccgatacctccgggcttttgctcatgttcgcctcatagggtcatctgggtggttgcctaaggaaaagtatgtcatggagactaacttgcttggcattgaataaaaggtgagttgagagtggagcgtgtttaaattgcaatcctgcctctatttctgtgcttgcagggaacagtcatccttaattgctatcctccatcatcatcatgattatttctggtttttctctggttgcggagaatccatactccaggtattccaatgtctcagcattgccaggcctgtctgagcgtcaggatgtaggtagtctgggctctctgccttctattcttgtccaggatactctgccaaaagaatcatgttgtggctgccacccctcccacaaagcctcccgcttgggtcagtccaggactggagttgggtatggactgttcatgtctatccactgctacgtcagggcaacacccactgagagtgaccttgtagactgcagtgggagacacccttcaaaacctctcctctcctgtcctgagagccaggttaaaaccatcagccccgcatcctgagtgcaaacttttcctaaccctgctgctaagctagacacctcacttactgagagccagcatgtccaccgctgtgctggagaaccctgggctggggaggaaactgtcagacttcgggcaggagacttcatacattgaggataactgtaaccagaatggcgccatctctctgatcttcagcctgaaggaggaagtgggcgccctggcaaaggtgctgcgcctgtttgaggagaacgacgtgaatctgacccacatcgagtcccggccttctagactgaagaaggacgagtacgagttctttacccacctggataagcggtccctgccagccctgacaaacatcatcaagatcctgaggcacgacatcggagcaaccgtgcacgagctgtctcgggacaagaagaaggataccgtgccctggttccctcggacaatccaggagctggatagatttgccaaccagatcctgtcttacggagcagagctggacgcagatcaccctggcttcaaggacccagtgtatcgggcccggagaaagcagtttgccgatatcgcctacaattataggcacggacagccaatccctcgcgtggagtatatggaggaggagaagaagacctggggcacagtgttcaagaccctgaagagcctgtacaagacacacgcctgctacgagtataaccacatcttccccctgctggagaagtattgtggctttcacgaggacaatatccctcagctggaggacgtgagccagttcctgcagacctgcacaggctttaggctgaggccagtggcaggactgctgagctcccgggacttcctgggaggactggccttcagagtgtttcactgcacccagtacatcaggcacggctccaagccaatgtatacaccagagcccgacatctgtcacgagctgctgggccacgtgcccctgtttagcgatagatccttcgcccagttttcccaggagatcggactggcatctctgggagcacctgacgagtacatcgagaagctggccaccatctattggttcacagtggagtttggcctgtgcaagcagggcgatagcatcaaggcctacggagcaggactgctgtctagcttcggcgagctgcagtattgtctgtccgagaagccaaagctgctgcccctggagctggagaagaccgccatccagaactacaccgtgacagagttccagcccctgtactatgtggccgagtcttttaacgatgccaaggagaaggtgagaaatttcgccgccacaatccctaggcccttcagcgtgcggtacgacccttatacccagaggatcgaggtgctggataatacacagcagctgaagatcctggctgactcaatcaatagcgaaatcggaatcctgtgctccgccctgcagaaaatcaaatgagaattcaaggcctctcgagcctctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaagctttacgtacgatcgtcgagcagctgttgtcctggagaacggagtcctgagcagaaaactctcagactttgggcaggtaagcctgttgggcttccactgctaggagagaattggttccccacatgtgaaagcagtctgggaaatgctggtatttccagtctcctaaggctactaagaaatatgactttatttagaggcgaggaaaatgcccaggaagtcaactgatgagactagtcttaacaagttgaggatacagaaagttggggatctgagctgctaccaacatctgtgtgtctttgggtggctcattggtatcctctgcctattggctttatcttctgtacactgaaaggaaatggctggtccttagtcacctggggtgggagtccctatctctccagggatacttattcaatcctttcttctgggtatcaaaatgacaagcttgtaagaaactgtcctctttcggctttcaggaggtgatgtcgcatgaagagaatttggggggggggacttactcagaaccaaggagggagaaattaaacagagagggaaatgaacaggagttagcccggagcctgaagcaccttggggattatgctgggggtggagggaatccattgtcctccctagggagggcttgcagaacatgttcttttctgtgatatttgtactttccccagattgcaaatcatggtttgtacactgagattcagtctctggaggtaatatgccttttctagcttttccttggacaggactaaggggttgagggttgcctggagtcagagaaatttgtgttaaagaaggttgatatgaaacctgcaggtctagatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 71 - DNA, 4075 bp, Artificial Sequence
  Synthetic polynucleotide
ccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagcctcgaggaacgttttattttcccagagacaggctcagggacttctgggtgtagtggctgtgcagggcttcgtggagcacgctgcagctaaacacgttgccctcttgccatcggctcttgtccacggtcagccgtgaatacaggaagaagctgccatcgctgtccagcactggaggggtcgtcttgtagttgttttcaggctgtccgttgctctcccattcgacggcgatgtcgctggggtagaagcccttcaccaggcaggtcagagacacttggttctttgtcatttcttcttgagaaggaggcagtgtatagacttggggctctctgggctggcccttggcctttgagatggtcttctcgatgctgctaggcaggcccttgttagacaccttgcacttgtactctttgccgttcagccagtcctggtgcagcactgtcagcacggacaccacccggtaggtgctgttaaactgttcctcgcggggtttggtcttggcattatgaacttccactccgtcaacgtaccaattaaactgcacctcaggatcctcctgggacacatccaccaccacacatgtcacttcaggggtccggctgatcatcagggtgtctttgggctttggtgggaacaggaaaacagaagggccggcgactggtggagctggacatggtgggcattccacgcagcactttctctcaacggtcttatccactttggtgttgctaggcttgtggtccacattacaggtgtaggtctgtgtgccgaagttggagctaggaacggtgaccacgctactcagagaatacaggccagagctctgcagcacggctgggaaggtgtgcacgccgcttgtcagggcgccgctgttccagctcactgtcacgggttcggggaagtagtctttgaccagacaacccagggcggctgtgctttcagatgtgcttctgctgcatggggccagtgggaacacgctgggtcccttagtactggcgctgctgacggtgacaagggtgccttggccccacacgtcgaagtaccagttaggggatgagccaaaaaagtatctggcgcagtagtacacagctgtgtcctcgcttctcaggctgctcagttccatgtacacggtgcttgtggaggtatctctggtcatggtcactctatccttgaagttctctgtgtactcggtgtggccagatcctggcaggatctcgcccatccattccaggccttggccaggggcctgccgcacccactggatccagtagttgctgaagatgtggccgctggccttgcaggacaccttcacgctagctccgggctttttcacttcggctccgctttgaaccagctgcacttggctgtgcacgcctgtggcggtggccaccaggaacaggatgatgcaggaccagcccatggtggcactagttcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggccgcggcacgtgcttaaggcccccttttgcatccagtttattcctacatttgtcacactgttaacagcccaccccttccaatgagaccagtggtatcagtgagttgtggagatcaggaaaagggctcaagagaaaggcagtcaaagccctttttctgtccctgtcccagctgctttaataagatctccataagagaagagggacagctatgactgggagtagtcaggagaggaggaaaaatctggctagtaaaacatgtaaggaaaattttagggatgttaaagaaaaaaataacacaaaacaaaatataaaaaaaatctaacctcaagtcaaggcttttctatggaataaggaatggacagcagggggctgtttcatatactgatgacctctttatagccaacctttgttcatggcagccagcatatgggcatatgttgccaaactctaaaccaaatactcattctgatgttttaaatgatttgccctcccatatgtccttccgagtgagagacacaaaaaattccaacacactattgcaatgaaaataaatttcctttattagccagaagtcagatgctcaaggggcttcatgatgtccccataatttttggcagagggaaaaagatctccggagggggaggctgctggtgaatattaaccaaggtcaccccagttatcggaggagcaaacaggggctaagtccacctcgagccatggcgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagcgccggcaagaggtaagggtttaagggatggttggttggtggggtattaatgtttaattacctggagcacctgcctgaaatcactttttttcaggttggaccggtgccaccatggacatgagggtccctgctcagctgctggggctcctgctgctctggctcagcggtgccagatgtgatatccagatgacccagtctccatctagcctgtccgccagcgtgggcgacagagtgaccatcacctgcggcgccagcgagaacatctatggcgctctgaactggtaccagcagaaacctggcaaggcccctaagctgctgatctacggcgccaccaacctggccgatggcgtgcctagtagattcagcggatctggcagcggcacagacttcaccctgaccatcagcagcctgcaacctgaggactttgccacatactactgccagaacgtgctgaatacacctctgacattcggccaaggaaccaaagtggaaatcaagcggaccgtggccgctcctagcgtgttcatcttccctccttccgatgaacaactgaagagcggaaccgcctctgtggtgtgcctgctgaacaacttctaccctagagaggccaaggtgcagtggaaggtcgacaacgccctgcagagcggcaacagccaggagagcgtgacggaacaggacagcaaggacagcacctacagcctgagctccacccttacactgtctaaagccgactacgagaagcacaaggtgtacgcctgtgaagtgacacaccagggcctgagcagccctgtgaccaagtcttttaaccggggcgagtgctgaattcgaatcgtacctagggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 72 - DNA, 2211 bp, Artificial Sequence
  Synthetic polynucleotide
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcgcggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctagagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 73 - DNA, 4093 bp, Artificial Sequence
  Synthetic polynucleotide
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgacttaaaccaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcgcggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctagagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 74 - DNA, 1353 bp, Artificial Sequence
  Synthetic polynucleotide
tccaccgctgtgctggagaaccctgggctggggaggaaactgtcagacttcgggcaggagacttcatacattgaggataactgtaaccagaatggcgccatctctctgatcttcagcctgaaggaggaagtgggcgccctggcaaaggtgctgcgcctgtttgaggagaacgacgtgaatctgacccacatcgagtcccggccttctagactgaagaaggacgagtacgagttctttacccacctggataagcggtccctgccagccctgacaaacatcatcaagatcctgaggcacgacatcggagcaaccgtgcacgagctgtctcgggacaagaagaaggataccgtgccctggttccctcggacaatccaggagctggatagatttgccaaccagatcctgtcttacggagcagagctggacgcagatcaccctggcttcaaggacccagtgtatcgggcccggagaaagcagtttgccgatatcgcctacaattataggcacggacagccaatccctcgcgtggagtatatggaggaggagaagaagacctggggcacagtgttcaagaccctgaagagcctgtacaagacacacgcctgctacgagtataaccacatcttccccctgctggagaagtattgtggctttcacgaggacaatatccctcagctggaggacgtgagccagttcctgcagacctgcacaggctttaggctgaggccagtggcaggactgctgagctcccgggacttcctgggaggactggccttcagagtgtttcactgcacccagtacatcaggcacggctccaagccaatgtatacaccagagcccgacatctgtcacgagctgctgggccacgtgcccctgtttagcgatagatccttcgcccagttttcccaggagatcggactggcatctctgggagcacctgacgagtacatcgagaagctggccaccatctattggttcacagtggagtttggcctgtgcaagcagggcgatagcatcaaggcctacggagcaggactgctgtctagcttcggcgagctgcagtattgtctgtccgagaagccaaagctgctgcccctggagctggagaagaccgccatccagaactacaccgtgacagagttccagcccctgtactatgtggccgagtcttttaacgatgccaaggagaaggtgagaaatttcgccgccacaatccctaggcccttcagcgtgcggtacgacccttatacccagaggatcgaggtgctggataatacacagcagctgaagatcctggctgactcaatcaatagcgaaatcggaatcctgtgctccgccctgcagaaaatcaaa

SEQ ID NO: 75 - DNA, 3939 bp, Artificial Sequence
  Synthetic polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcatatgcggccgcttcaggagcagttgtgcgaatagctggagaacaccaggctggatttaaacccagatcgctcttacatttgctctttacctgctgtgctcagcgttcacgtgccctctagctgtagttttctgaagtcagcgcacagcaaggcagtgtgcttagaggttaacagaagggaaaacaacaacaacaaaaatctaaatgagaatcctgactgtttcagctgggggtaaggggggcggattattcatataattgttataccagacggtcgcaggcttagtccaattgcagagaactcgcttcccaggcttctgagagtcccggaagtgcctaaacctgtctaatcgacggggcttgggtggcccgtcgctccctggcttcttccctttacccagggcgggcagcgaagtggtgcctcctgcgtcccccacaccctccctcagcccctcccctccggcccgtcctgggcaggtgacctggagcatccggcaggctgccctggcctcctgcgtcaggacaacgcccacgaggggcgttactgtgcggagatgcaccacgcaagagacaccctttgtaactctcttctcctccctagtgcgaggttaaaaccttcagccccacgtgctgtttgcaaacctgcctgtacctgaggccctaaaaagccagagacctcactcccggggagccagcatgtccactgcggtcctggaaaacccaggcttgggcaggaaactctctgactttggacaggtgagccacggcagcctgagctgctcagttaggggaatttgggcctccagagaaagagatctgaagactgctggtgcttcctggtttcataagctcagtaagaagtctgaattcgttggaagctgatgagaatatccaggaagtcaacagacaaatgtcctcaacaattgtttctaagtaggagaacatctgtcctcggtggctttcacaggaaaagcttctgacctcttctcttcctcccacagggcggtaccagatctggcagcggagagggcagaggaagtcttctaacatgcggtgacgtggaggagaatcccggcccttccaccgctgtgctggagaaccctgggctggggaggaaactgtcagacttcgggcaggagacttcatacattgaggataactgtaaccagaatggcgccatctctctgatcttcagcctgaaggaggaagtgggcgccctggcaaaggtgctgcgcctgtttgaggagaacgacgtgaatctgacccacatcgagtcccggccttctagactgaagaaggacgagtacgagttctttacccacctggataagcggtccctgccagccctgacaaacatcatcaagatcctgaggcacgacatcggagcaaccgtgcacgagctgtctcgggacaagaagaaggataccgtgccctggttccctcggacaatccaggagctggatagatttgccaaccagatcctgtcttacggagcagagctggacgcagatcaccctggcttcaaggacccagtgtatcgggcccggagaaagcagtttgccgatatcgcctacaattataggcacggacagccaatccctcgcgtggagtatatggaggaggagaagaagacctggggcacagtgttcaagaccctgaagagcctgtacaagacacacgcctgctacgagtataaccacatcttccccctgctggagaagtattgtggctttcacgaggacaatatccctcagctggaggacgtgagccagttcctgcagacctgcacaggctttaggctgaggccagtggcaggactgctgagctcccgggacttcctgggaggactggccttcagagtgtttcactgcacccagtacatcaggcacggctccaagccaatgtatacaccagagcccgacatctgtcacgagctgctgggccacgtgcccctgtttagcgatagatccttcgcccagttttcccaggagatcggactggcatctctgggagcacctgacgagtacatcgagaagctggccaccatctattggttcacagtggagtttggcctgtgcaagcagggcgatagcatcaaggcctacggagcaggactgctgtctagcttcggcgagctgcagtattgtctgtccgagaagccaaagctgctgcccctggagctggagaagaccgccatccagaactacaccgtgacagagttccagcccctgtactatgtggccgagtcttttaacgatgccaaggagaaggtgagaaatttcgccgccacaatccctaggcccttcagcgtgcggtacgacccttatacccagaggatcgaggtgctggataatacacagcagctgaagatcctggctgactcaatcaatagcgaaatcggaatcctgtgctccgccctgcagaaaatcaaatgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaactgggatgggatgtggaatccttctagatttcttttgtaatatttataaagtgctctcagcaaggtatcaaaatggcaaaattgtgagtaactatcctcctttcattttgggaagaagatgaggcatgaagagaattcagacagaaacttactcagaccaggggaggcagaaactaagcagagaggaaaatgaccaagagttagccctgggcatggaatgtgaaagaaccctaaacgtgacttggaaataatgcccaaggtatattccattctccgggatttgttggcattttcttgaggtgaagaattgcagaatacattctttaatgtgacctacatatttacccatgggaggaagtctgctcctggactcttgagattcagtcataaagcccaggccagggaaataatgtaagtctgcaggcccctgtcatcagtaggattagggagaagagttctcagtagaaaacagggaggctggagagaaaagaatggttaatgttaacgttaatataactagaaagactgcagaacttaggactgatttttatttgaatccttaaaaaaaaaatttcttatgaaaatagtacatggctcttaggagacagaacttattgtacagaggaacagcgtgagagtcagagtgatcccagaacaggtcctggctccatcctgcacatagttttggtgctgctggcaatacggtccccacaactgtgggaaggggttaggggcagggatctcatcaggaaagcataggggtttaaagttctttatagagcacttagaagattgagaatccacaaattatattaataacaaacaaagtagtgtcgtgttatatagtaaatgtgaatttgcagacacatttagggaaaagttataattaaaaaaataggctgtatatatacctgcaggtctagatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 76 - DNA, 2211 bp, Artificial Sequence
  Synthetic polynucleotide
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgataagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctagagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 77 - DNA, 4093 bp, Artificial Sequence
  Synthetic polynucleotide
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgacttaaaccaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgataagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctagagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 78 - DNA, 2356 bp, Artificial Sequence
  Synthetic polynucleotide
ctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacctccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcctctaaggtaaatataaaatttttaagtgtataatgtgttaaactactgattctaattgtttctctcttttagattccaacctttggaactgaccgccaccatgtccaccgctgtgctggagaaccctgggctggggaggaaactgtcagacttcgggcaggagacttcatacattgaggataactgtaaccagaatggcgccatctctctgatcttcagcctgaaggaggaagtgggcgccctggcaaaggtgctgcgcctgtttgaggagaacgacgtgaatctgacccacatcgagtcccggccttctagactgaagaaggacgagtacgagttctttacccacctggataagcggtccctgccagccctgacaaacatcatcaagatcctgaggcacgacatcggagcaaccgtgcacgagctgtctcgggacaagaagaaggataccgtgccctggttccctcggacaatccaggagctggatagatttgccaaccagatcctgtcttacggagcagagctggacgcagatcaccctggcttcaaggacccagtgtatcgggcccggagaaagcagtttgccgatatcgcctacaattataggcacggacagccaatccctcgcgtggagtatatggaggaggagaagaagacctggggcacagtgttcaagaccctgaagagcctgtacaagacacacgcctgctacgagtataaccacatcttccccctgctggagaagtattgtggctttcacgaggacaatatccctcagctggaggacgtgagccagttcctgcagacctgcacaggctttaggctgaggccagtggcaggactgctgagctcccgggacttcctgggaggactggccttcagagtgtttcactgcacccagtacatcaggcacggctccaagccaatgtatacaccagagcccgacatctgtcacgagctgctgggccacgtgcccctgtttagcgatagatccttcgcccagttttcccaggagatcggactggcatctctgggagcacctgacgagtacatcgagaagctggccaccatctattggttcacagtggagtttggcctgtgcaagcagggcgatagcatcaaggcctacggagcaggactgctgtctagcttcggcgagctgcagtattgtctgtccgagaagccaaagctgctgcccctggagctggagaagaccgccatccagaactacaccgtgacagagttccagcccctgtactatgtggccgagtcttttaacgatgccaaggagaaggtgagaaatttcgccgccacaatccctaggcccttcagcgtgcggtacgacccttatacccagaggatcgaggtgctggataatacacagcagctgaagatcctggctgactcaatcaatagcgaaatcggaatcctgtgctccgccctgcagaaaatcaaatgaatgctttatttgtgaaatttgtgatgctattgctttatttgtaaccattataagctgcaataaacaagttaacaacaacaattgcattcattttatgtttcaggttcagggggaggtgtgggaggttttttaaagcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 79 - DNA, 1359 bp, Artificial Sequence
  Synthetic polynucleotide
atgtccaccgctgtgctggagaaccctgggctggggaggaaactgtcagacttcgggcaggagacttcatacattgaggataactgtaaccagaatggcgccatctctctgatcttcagcctgaaggaggaagtgggcgccctggcaaaggtgctgcgcctgtttgaggagaacgacgtgaatctgacccacatcgagtcccggccttctagactgaagaaggacgagtacgagttctttacccacctggataagcggtccctgccagccctgacaaacatcatcaagatcctgaggcacgacatcggagcaaccgtgcacgagctgtctcgggacaagaagaaggataccgtgccctggttccctcggacaatccaggagctggatagatttgccaaccagatcctgtcttacggagcagagctggacgcagatcaccctggcttcaaggacccagtgtatcgggcccggagaaagcagtttgccgatatcgcctacaattataggcacggacagccaatccctcgcgtggagtatatggaggaggagaagaagacctggggcacagtgttcaagaccctgaagagcctgtacaagacacacgcctgctacgagtataaccacatcttccccctgctggagaagtattgtggctttcacgaggacaatatccctcagctggaggacgtgagccagttcctgcagacctgcacaggctttaggctgaggccagtggcaggactgctgagctcccgggacttcctgggaggactggccttcagagtgtttcactgcacccagtacatcaggcacggctccaagccaatgtatacaccagagcccgacatctgtcacgagctgctgggccacgtgcccctgtttagcgatagatccttcgcccagttttcccaggagatcggactggcatctctgggagcacctgacgagtacatcgagaagctggccaccatctattggttcacagtggagtttggcctgtgcaagcagggcgatagcatcaaggcctacggagcaggactgctgtctagcttcggcgagctgcagtattgtctgtccgagaagccaaagctgctgcccctggagctggagaagaccgccatccagaactacaccgtgacagagttccagcccctgtactatgtggccgagtcttttaacgatgccaaggagaaggtgagaaatttcgccgccacaatccctaggcccttcagtgtgcgttacgacccttatacccagaggatcgaggtgctggataatacacagcagctgaagatcctggctgactcaatcaatagcgaaatcggaatcctgtgctccgccctgcagaaaatcaaatga

SEQ ID NO: 80 - DNA, 4452 bp, Artificial Sequence
  Synthetic polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcatatgcggccgcttcaggagcagttgtgcgaatagctggagaacaccaggctggatttaaacccagatcgctcttacatttgctctttacctgctgtgctcagcgttcacgtgccctctagctgtagttttctgaagtcagcgcacagcaaggcagtgtgcttagaggttaacagaagggaaaacaacaacaacaaaaatctaaatgagaatcctgactgtttcagctgggggtaaggggggcggattattcatataattgttataccagacggtcgcaggcttagtccaattgcagagaactcgcttcccaggcttctgagagtcccggaagtgcctaaacctgtctaatcgacggggcttgggtggcccgtcgctccctggcttcttccctttacccagggcgggcagcgaagtggtgcctcctgcgtcccccacaccctccctcagcccctcccctccggcccgtcctgggcaggtgacctggagcatccggcaggctgccctggcctcctgcgtcaggacaacgcccacgaggggcgttactgtgcggagatgcaccacgcaagagacaccctttgtaactctcttctcctccctagtgcgaggttaaaaccttcagccccacgtgctgtttgcaaacctgcctgtacctgaggccctaaaaagccagagacctcactcccggggagccagcatgtccactgcggtcctggaaaacccaggcttgggcaggaaactctctgactttggacaggtgagccacggcagcctgagctgctcagttaggggaatttgggcctccagagaaagagatctgaagactgctggtgcttcctggtttcataagctcagtaagaagtctgaattcgttggaagctgatgagaatatccaggaagtcaacagacaaatgtcctcaacaattgtttctaagtaggagaacatctgtcctcggtggctttcacaggaaccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcctctaaggtaaatataaaatttttaagtgtataatgtgttaaactactgattctaattgtttctctcttttagattccaacctttggaactgaccgccaccatgtccaccgctgtgctggagaaccctgggctggggaggaaactgtcagacttcgggcaggagacttcatacattgaggataactgtaaccagaatggcgccatctctctgatcttcagcctgaaggaggaagtgggcgccctggcaaaggtgctgcgcctgtttgaggagaacgacgtgaatctgacccacatcgagtcccggccttctagactgaagaaggacgagtacgagttctttacccacctggataagcggtccctgccagccctgacaaacatcatcaagatcctgaggcacgacatcggagcaaccgtgcacgagctgtctcgggacaagaagaaggataccgtgccctggttccctcggacaatccaggagctggatagatttgccaaccagatcctgtcttacggagcagagctggacgcagatcaccctggcttcaaggacccagtgtatcgggcccggagaaagcagtttgccgatatcgcctacaattataggcacggacagccaatccctcgcgtggagtatatggaggaggagaagaagacctggggcacagtgttcaagaccctgaagagcctgtacaagacacacgcctgctacgagtataaccacatcttccccctgctggagaagtattgtggctttcacgaggacaatatccctcagctggaggacgtgagccagttcctgcagacctgcacaggctttaggctgaggccagtggcaggactgctgagctcccgggacttcctgggaggactggccttcagagtgtttcactgcacccagtacatcaggcacggctccaagccaatgtatacaccagagcccgacatctgtcacgagctgctgggccacgtgcccctgtttagcgatagatccttcgcccagttttcccaggagatcggactggcatctctgggagcacctgacgagtacatcgagaagctggccaccatctattggttcacagtggagtttggcctgtgcaagcagggcgatagcatcaaggcctacggagcaggactgctgtctagcttcggcgagctgcagtattgtctgtccgagaagccaaagctgctgcccctggagctggagaagaccgccatccagaactacaccgtgacagagttccagcccctgtactatgtggccgagtcttttaacgatgccaaggagaaggtgagaaatttcgccgccacaatccctaggcccttcagtgtgcgttacgacccttatacccagaggatcgaggtgctggataatacacagcagctgaagatcctggctgactcaatcaatagcgaaatcggaatcctgtgctccgccctgcagaaaatcaaatgagaattcaaggcctctcgagcctctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaagctttacgtacgatcgtcgactgggatgggatgtggaatccttctagatttcttttgtaatatttataaagtgctctcagcaaggtatcaaaatggcaaaattgtgagtaactatcctcctttcattttgggaagaagatgaggcatgaagagaattcagacagaaacttactcagaccaggggaggcagaaactaagcagagaggaaaatgaccaagagttagccctgggcatggaatgtgaaagaaccctaaacgtgacttggaaataatgcccaaggtatattccattctccgggatttgttggcattttcttgaggtgaagaattgcagaatacattctttaatgtgacctacatatttacccatgggaggaagtctgctcctggactcttgagattcagtcataaagcccaggccagggaaataatgtaagtctgcaggcccctgtcatcagtaggattagggagaagagttctcagtagaaaacagggaggctggagagaaaagaatggttaatgttaacgttaatataactagaaagactgcagaacttaggactgatttttatttgaatccttaaaaaaaaaatttcttatgaaaatagtacatggctcttaggagacagaacttattgtacagaggaacagcgtgagagtcagagtgatcccagaacaggtcctggctccatcctgcacatagttttggtgctgctggcaatacggtccccacaactgtgggaaggggttaggggcagggatctcatcaggaaagcataggggtttaaagttctttatagagcacttagaagattgagaatccacaaattatattaataacaaacaaagtagtgtcgtgttatatagtaaatgtgaatttgcagacacatttagggaaaagttataattaaaaaaataggctgtatatatacctgcaggtctagatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 81 - DNA, 1735 bp, Artificial Sequence
  Synthetic polynucleotide
atgcccccacccaggaccggaagaggcctgctgtggctgggcctggtgctctcttccgtgtgcgtggccctgggaagcgaaacccaggccaacagcacaaccgacgccctgaatgtgctgctgatcattgtggacgatctgagaccctccctgggctgttacggcgacaaactggtgcggtccccaaacatcgaccagctggcctcccactccctgctgttccagaacgccttcgcccagcaggccgtgtgtgcccccagcagggtgagcttcctgaccggcagaagacctgacaccaccaggctgtacgactttaacagctactggcgggtgcacgccggcaatttcagcaccattcctcagtacttcaaggagaatggctacgtgacaatgtccgtgggcaaggtgtttcatcccggcattagctccaaccacaccgacgatagcccatactcctggtccttccccccctaccatccctccagcgagaagtacgagaacaccaaaacctgcagaggccctgacggagagctgcacgccaacctgctgtgccctgtggatgtcctggatgtgcccgaaggtaagggtttaagggatggttggttggtggggtattaatgtttaattacctggagcacctgcctgaaatcactttttttcaggcaccctgccagacaagcagtccacagagcaggccatccagctgctggagaagatgaagacaagcgccagccccttctttctggccgtgggataccacaagcctcacattccattccggtacccaaaagagttccagaagctgtaccctctggaaaacatcaccctggcccctgaccccgaggtgccagacgggctgcctcctgtggcctacaatccctggatggacatcagacagcgggaggatgtgcaggccctgaatatttccgtgccctatgggcccatccctgtggactttcagcggaaaatcagacagtcttactttgccagcgtgtcctacctggacacccaggtgggccgcctgctctcagccctggacgacctgcagctggccaattccaccatcatcgccttcaccagcgatcacggctgggccctgggcgagcacggggagtgggccaaatacagcaacttcgatgtggccacccacgtgcctctgattttttatgtgcccggccggacagccagcctgcccgaggccggggagaagctctttccttacctggaccctttcgactctgccagccagctgatggaacctggcagacagagcatggacctggtggagctggtgagcctcttccccactctggccggcctggctggcctgcaggtgccaccaagatgcccagtgccttctttccacgtggagctgtgtagagagggaaagaacctgctgaagcacttcagatttagagatctggaggaggatccctacctgccaggcaacccaagggagctgatcgcctacagccagtatcccagaccctctgatatcccccagtggaacagcgataagccctccctgaaagacatcaagattatgggctactccatcaggaccattgactaccggtacacagtgtgggtgggcttcaaccccgatgagtttctggccaacttcagcgacatccacgccggcgagctgtattttgtggactccgaccccctgcaggaccacaacatgtacaacgactcccagggcggcgacctgttccagctgctgatgccctga

SEQ ID NO: 82 - DNA, 2521 bp, Artificial Sequence
  Synthetic polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcatatgcggccgcaactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtgccaccatgcccccacccaggaccggaagaggcctgctgtggctgggcctggtgctctcttccgtgtgcgtggccctgggaagcgaaacccaggccaacagcacaaccgacgccctgaatgtgctgctgatcattgtggacgatctgagaccctccctgggctgttacggcgacaaactggtgcggtccccaaacatcgaccagctggcctcccactccctgctgttccagaacgccttcgcccagcaggccgtgtgtgcccccagcagggtgagcttcctgaccggcagaagacctgacaccaccaggctgtacgactttaacagctactggcgggtgcacgccggcaatttcagcaccattcctcagtacttcaaggagaatggctacgtgacaatgtccgtgggcaaggtgtttcatcccggcattagctccaaccacaccgacgatagcccatactcctggtccttccccccctaccatccctccagcgagaagtacgagaacaccaaaacctgcagaggccctgacggagagctgcacgccaacctgctgtgccctgtggatgtcctggatgtgcccgaaggtaagggtttaagggatggttggttggtggggtattaatgtttaattacctggagcacctgcctgaaatcactttttttcaggcaccctgccagacaagcagtccacagagcaggccatccagctgctggagaagatgaagacaagcgccagccccttctttctggccgtgggataccacaagcctcacattccattccggtacccaaaagagttccagaagctgtaccctctggaaaacatcaccctggcccctgaccccgaggtgccagacgggctgcctcctgtggcctacaatccctggatggacatcagacagcgggaggatgtgcaggccctgaatatttccgtgccctatgggcccatccctgtggactttcagcggaaaatcagacagtcttactttgccagcgtgtcctacctggacacccaggtgggccgcctgctctcagccctggacgacctgcagctggccaattccaccatcatcgccttcaccagcgatcacggctgggccctgggcgagcacggggagtgggccaaatacagcaacttcgatgtggccacccacgtgcctctgattttttatgtgcccggccggacagccagcctgcccgaggccggggagaagctctttccttacctggaccctttcgactctgccagccagctgatggaacctggcagacagagcatggacctggtggagctggtgagcctcttccccactctggccggcctggctggcctgcaggtgccaccaagatgcccagtgccttctttccacgtggagctgtgtagagagggaaagaacctgctgaagcacttcagatttagagatctggaggaggatccctacctgccaggcaacccaagggagctgatcgcctacagccagtatcccagaccctctgatatcccccagtggaacagcgataagccctccctgaaagacatcaagattatgggctactccatcaggaccattgactaccggtacacagtgtgggtgggcttcaaccccgatgagtttctggccaacttcagcgacatccacgccggcgagctgtattttgtggactccgaccccctgcaggaccacaacatgtacaacgactcccagggcggcgacctgttccagctgctgatgccctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaacctgcaggtctagatacgtagataagtagcatggcgggttaatcattaactacaaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 83 - DNA, 3668 bp, Artificial Sequence
  Synthetic polynucleotide
ttttcccagagacaggctcagggacttctgggtgtagtggctgtgcagggcttcgtggagcacgctgcagctaaacacgttgccctcttgccatcggctcttgtccacggtcagccgtgaatacaggaagaagctgccatcgctgtccagcactggaggggtcgtcttgtagttgttttcaggctgtccgttgctctcccattcgacggcgatgtcgctggggtagaagcccttcaccaggcaggtcagagacacttggttctttgtcatttcttcttgagaaggaggcagtgtatagacttggggctctctgggctggcccttggcctttgagatggtcttctcgatgctgctaggcaggcccttgttagacaccttgcacttgtactctttgccgttcagccagtcctggtgcagcactgtcagcacggacaccacccggtaggtgctgttaaactgttcctcgcggggtttggtcttggcattatgaacttccactccgtcaacgtaccaattaaactgcacctcaggatcctcctgggacacatccaccaccacacatgtcacttcaggggtccggctgatcatcagggtgtctttgggctttggtgggaacaggaaaacagaagggccggcgactggtggagctggacatggtgggcattccacgcagcactttctctcaacggtcttatccactttggtgttgctaggcttgtggtccacattacaggtgtaggtctgtgtgccgaagttggagctaggaacggtgaccacgctactcagagaatacaggccagagctctgcagcacggctgggaaggtgtgcacgccgcttgtcagggcgccgctgttccagctcactgtcacgggttcggggaagtagtctttgaccagacaacccagggcggctgtgctttcagatgtgcttctgctgcatggggccagtgggaacacgctgggtcccttagtactggcgctgctgacggtgacaagggtgccttggccccacacgtcgaagtaccagttaggggatgagccaaaaaagtatctggcgcagtagtacacagctgtgtcctcgcttctcaggctgctcagttccatgtacacggtgcttgtggaggtatctctggtcatggtcactctatccttgaagttctctgtgtactcggtgtggccagatcctggcaggatctcgcccatccattccaggccttggccaggggcctgccgcacccactggatccagtagttgctgaagatgtggccgctggccttgcaggacaccttcacgctagctccgggctttttcacttcggctccgctttgaaccagctgcacttggctgtgcacgcctgtggcggtggccaccaggaacaggatgatgcaggaccagcccatggtggcactagttcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggccgcggcacgtgcttaaggcccccttttgcatccagtttattcctacatttgtcacactgttaacagcccaccccttccaatgagaccagtggtatcagtgagttgtggagatcaggaaaagggctcaagagaaaggcagtcaaagccctttttctgtccctgtcccagctgctttaataagatctccataagagaagagggacagctatgactgggagtagtcaggagaggaggaaaaatctggctagtaaaacatgtaaggaaaattttagggatgttaaagaaaaaaataacacaaaacaaaatataaaaaaaatctaacctcaagtcaaggcttttctatggaataaggaatggacagcagggggctgtttcatatactgatgacctctttatagccaacctttgttcatggcagccagcatatgggcatatgttgccaaactctaaaccaaatactcattctgatgttttaaatgatttgccctcccatatgtccttccgagtgagagacacaaaaaattccaacacactattgcaatgaaaataaatttcctttattagccagaagtcagatgctcaaggggcttcatgatgtccccataatttttggcagagggaaaaagatctccggagggggaggctgctggtgaatattaaccaaggtcaccccagttatcggaggagcaaacaggggctaagtccacctcgagccatggcgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagcgccggcaagaggtaagggtttaagggatggttggttggtggggtattaatgtttaattacctggagcacctgcctgaaatcactttttttcaggttggaccggtgccaccatggacatgagggtccctgctcagctgctggggctcctgctgctctggctcagcggtgccagatgtgatatccagatgacccagtctccatctagcctgtccgccagcgtgggcgacagagtgaccatcacctgcggcgccagcgagaacatctatggcgctctgaactggtaccagcagaaacctggcaaggcccctaagctgctgatctacggcgccaccaacctggccgatggcgtgcctagtagattcagcggatctggcagcggcacagacttcaccctgaccatcagcagcctgcaacctgaggactttgccacatactactgccagaacgtgctgaatacacctctgacattcggccaaggaaccaaagtggaaatcaagcggaccgtggccgctcctagcgtgttcatcttccctccttccgatgaacaactgaagagcggaaccgcctctgtggtgtgcctgctgaacaacttctaccctagagaggccaaggtgcagtggaaggtcgacaacgccctgcagagcggcaacagccaggagagcgtgacggaacaggacagcaaggacagcacctacagcctgagctccacccttacactgtctaaagccgactacgagaagcacaaggtgtacgcctgtgaagtgacacaccagggcctgagcagccctgtgaccaagtcttttaaccggggcgagtgc

SEQ ID NO: 84 - DNA, 4483 bp, Artificial Sequence
  Synthetic polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcacacgtgacgcgtccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagcctcgaggaacgttttattttcccagagacaggctcagggacttctgggtgtagtggctgtgcagggcttcgtggagcacgctgcagctaaacacgttgccctcttgccatcggctcttgtccacggtcagccgtgaatacaggaagaagctgccatcgctgtccagcactggaggggtcgtcttgtagttgttttcaggctgtccgttgctctcccattcgacggcgatgtcgctggggtagaagcccttcaccaggcaggtcagagacacttggttctttgtcatttcttcttgagaaggaggcagtgtatagacttggggctctctgggctggcccttggcctttgagatggtcttctcgatgctgctaggcaggcccttgttagacaccttgcacttgtactctttgccgttcagccagtcctggtgcagcactgtcagcacggacaccacccggtaggtgctgttaaactgttcctcgcggggtttggtcttggcattatgaacttccactccgtcaacgtaccaattaaactgcacctcaggatcctcctgggacacatccaccaccacacatgtcacttcaggggtccggctgatcatcagggtgtctttgggctttggtgggaacaggaaaacagaagggccggcgactggtggagctggacatggtgggcattccacgcagcactttctctcaacggtcttatccactttggtgttgctaggcttgtggtccacattacaggtgtaggtctgtgtgccgaagttggagctaggaacggtgaccacgctactcagagaatacaggccagagctctgcagcacggctgggaaggtgtgcacgccgcttgtcagggcgccgctgttccagctcactgtcacgggttcggggaagtagtctttgaccagacaacccagggcggctgtgctttcagatgtgcttctgctgcatggggccagtgggaacacgctgggtcccttagtactggcgctgctgacggtgacaagggtgccttggccccacacgtcgaagtaccagttaggggatgagccaaaaaagtatctggcgcagtagtacacagctgtgtcctcgcttctcaggctgctcagttccatgtacacggtgcttgtggaggtatctctggtcatggtcactctatccttgaagttctctgtgtactcggtgtggccagatcctggcaggatctcgcccatccattccaggccttggccaggggcctgccgcacccactggatccagtagttgctgaagatgtggccgctggccttgcaggacaccttcacgctagctccgggctttttcacttcggctccgctttgaaccagctgcacttggctgtgcacgcctgtggcggtggccaccaggaacaggatgatgcaggaccagcccatggtggcactagttcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggccgcggcacgtgcttaaggcccccttttgcatccagtttattcctacatttgtcacactgttaacagcccaccccttccaatgagaccagtggtatcagtgagttgtggagatcaggaaaagggctcaagagaaaggcagtcaaagccctttttctgtccctgtcccagctgctttaataagatctccataagagaagagggacagctatgactgggagtagtcaggagaggaggaaaaatctggctagtaaaacatgtaaggaaaattttagggatgttaaagaaaaaaataacacaaaacaaaatataaaaaaaatctaacctcaagtcaaggcttttctatggaataaggaatggacagcagggggctgtttcatatactgatgacctctttatagccaacctttgttcatggcagccagcatatgggcatatgttgccaaactctaaaccaaatactcattctgatgttttaaatgatttgccctcccatatgtccttccgagtgagagacacaaaaaattccaacacactattgcaatgaaaataaatttcctttattagccagaagtcagatgctcaaggggcttcatgatgtccccataatttttggcagagggaaaaagatctccggagggggaggctgctggtgaatattaaccaaggtcaccccagttatcggaggagcaaacaggggctaagtccacctcgagccatggcgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagcgccggcaagaggtaagggtttaagggatggttggttggtggggtattaatgtttaattacctggagcacctgcctgaaatcactttttttcaggttggaccggtgccaccatggacatgagggtccctgctcagctgctggggctcctgctgctctggctcagcggtgccagatgtgatatccagatgacccagtctccatctagcctgtccgccagcgtgggcgacagagtgaccatcacctgcggcgccagcgagaacatctatggcgctctgaactggtaccagcagaaacctggcaaggcccctaagctgctgatctacggcgccaccaacctggccgatggcgtgcctagtagattcagcggatctggcagcggcacagacttcaccctgaccatcagcagcctgcaacctgaggactttgccacatactactgccagaacgtgctgaatacacctctgacattcggccaaggaaccaaagtggaaatcaagcggaccgtggccgctcctagcgtgttcatcttccctccttccgatgaacaactgaagagcggaaccgcctctgtggtgtgcctgctgaacaacttctaccctagagaggccaaggtgcagtggaaggtcgacaacgccctgcagagcggcaacagccaggagagcgtgacggaacaggacagcaaggacagcacctacagcctgagctccacccttacactgtctaaagccgactacgagaagcacaaggtgtacgcctgtgaagtgacacaccagggcctgagcagccctgtgaccaagtcttttaaccggggcgagtgctgaattcgaatcgtacctagggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaagcttgtttaaacgtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 85 - DNA, 3533 bp, Artificial Sequence
  Synthetic polynucleotide
gcttcaggagcagttgtgcgaatagctggagaacaccaggctggatttaaacccagatcgctcttacatttgctctttacctgctgtgctcagcgttcacgtgccctctagctgtagttttctgaagtcagcgcacagcaaggcagtgtgcttagaggttaacagaagggaaaacaacaacaacaaaaatctaaatgagaatcctgactgtttcagctgggggtaaggggggcggattattcatataattgttataccagacggtcgcaggcttagtccaattgcagagaactcgcttcccaggcttctgagagtcccggaagtgcctaaacctgtctaatcgacggggcttgggtggcccgtcgctccctggcttcttccctttacccagggcgggcagcgaagtggtgcctcctgcgtcccccacaccctccctcagcccctcccctccggcccgtcctgggcaggtgacctggagcatccggcaggctgccctggcctcctgcgtcaggacaacgcccacgaggggcgttactgtgcggagatgcaccacgcaagagacaccctttgtaactctcttctcctccctagtgcgaggttaaaaccttcagccccacgtgctgtttgcaaacctgcctgtacctgaggccctaaaaagccagagacctcactcccggggagccagcatgtccactgcggtcctggaaaacccaggcttgggcaggaaactctctgactttggacaggtgagccacggcagcctgagctgctcagttaggggaatttgggcctccagagaaagagatctgaagactgctggtgcttcctggtttcataagctcagtaagaagtctgaattcgttggaagctgatgagaatatccaggaagtcaacagacaaatgtcctcaacaattgtttctaagtaggagaacatctgtcctcggtggctttcacaggaaaagcttctgacctcttctcttcctcccacagggcggtaccagatctggcagcggagagggcagaggaagtcttctaacatgcggtgacgtggaggagaatcccggcccttccaccgctgtgctggagaaccctgggctggggaggaaactgtcagacttcgggcaggagacttcatacattgaggataactgtaaccagaatggcgccatctctctgatcttcagcctgaaggaggaagtgggcgccctggcaaaggtgctgcgcctgtttgaggagaacgacgtgaatctgacccacatcgagtcccggccttctagactgaagaaggacgagtacgagttctttacccacctggataagcggtccctgccagccctgacaaacatcatcaagatcctgaggcacgacatcggagcaaccgtgcacgagctgtctcgggacaagaagaaggataccgtgccctggttccctcggacaatccaggagctggatagatttgccaaccagatcctgtcttacggagcagagctggacgcagatcaccctggcttcaaggacccagtgtatcgggcccggagaaagcagtttgccgatatcgcctacaattataggcacggacagccaatccctcgcgtggagtatatggaggaggagaagaagacctggggcacagtgttcaagaccctgaagagcctgtacaagacacacgcctgctacgagtataaccacatcttccccctgctggagaagtattgtggctttcacgaggacaatatccctcagctggaggacgtgagccagttcctgcagacctgcacaggctttaggctgaggccagtggcaggactgctgagctcccgggacttcctgggaggactggccttcagagtgtttcactgcacccagtacatcaggcacggctccaagccaatgtatacaccagagcccgacatctgtcacgagctgctgggccacgtgcccctgtttagcgatagatccttcgcccagttttcccaggagatcggactggcatctctgggagcacctgacgagtacatcgagaagctggccaccatctattggttcacagtggagtttggcctgtgcaagcagggcgatagcatcaaggcctacggagcaggactgctgtctagcttcggcgagctgcagtattgtctgtccgagaagccaaagctgctgcccctggagctggagaagaccgccatccagaactacaccgtgacagagttccagcccctgtactatgtggccgagtcttttaacgatgccaaggagaaggtgagaaatttcgccgccacaatccctaggcccttcagcgtgcggtacgacccttatacccagaggatcgaggtgctggataatacacagcagctgaagatcctggctgactcaatcaatagcgaaatcggaatcctgtgctccgccctgcagaaaatcaaatgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaactgggatgggatgtggaatccttctagatttcttttgtaatatttataaagtgctctcagcaaggtatcaaaatggcaaaattgtgagtaactatcctcctttcattttgggaagaagatgaggcatgaagagaattcagacagaaacttactcagaccaggggaggcagaaactaagcagagaggaaaatgaccaagagttagccctgggcatggaatgtgaaagaaccctaaacgtgacttggaaataatgcccaaggtatattccattctccgggatttgttggcattttcttgaggtgaagaattgcagaatacattctttaatgtgacctacatatttacccatgggaggaagtctgctcctggactcttgagattcagtcataaagcccaggccagggaaataatgtaagtctgcaggcccctgtcatcagtaggattagggagaagagttctcagtagaaaacagggaggctggagagaaaagaatggttaatgttaacgttaatataactagaaagactgcagaacttaggactgatttttatttgaatccttaaaaaaaaaatttcttatgaaaatagtacatggctcttaggagacagaacttattgtacagaggaacagcgtgagagtcagagtgatcccagaacaggtcctggctccatcctgcacatagttttggtgctgctggcaatacggtccccacaactgtgggaaggggttaggggcagggatctcatcaggaaagcataggggtttaaagttctttatagagcacttagaagattgagaatccacaaattatattaataacaaacaaagtagtgtcgtgttatatagtaaatgtgaatttgcagacacatttagggaaaagttataattaaaaaaataggctgtatatata

SEQ ID NO: 86 - DNA, 4061 bp, Artificial Sequence
  Synthetic polynucleotide
tgcggccgcttcaggagcagttgtgcgaatagctggagaacaccaggctggatttaaacccagatcgctcttacatttgctctttacctgctgtgctcagcgttcacgtgccctctagctgtagttttctgaagtcagcgcacagcaaggcagtgtgcttagaggttaacagaagggaaaacaacaacaacaaaaatctaaatgagaatcctgactgtttcagctgggggtaaggggggcggattattcatataattgttataccagacggtcgcaggcttagtccaattgcagagaactcgcttcccaggcttctgagagtcccggaagtgcctaaacctgtctaatcgacggggcttgggtggcccgtcgctccctggcttcttccctttacccagggcgggcagcgaagtggtgcctcctgcgtcccccacaccctccctcagcccctcccctccggcccgtcctgggcaggtgacctggagcatccggcaggctgccctggcctcctgcgtcaggacaacgcccacgaggggcgttactgtgcggagatgcaccacgcaagagacaccctttgtaactctcttctcctccctagtgcgaggttaaaaccttcagccccacgtgctgtttgcaaacctgcctgtacctgaggccctaaaaagccagagacctcactcccggggagccagcatgtccactgcggtcctggaaaacccaggcttgggcaggaaactctctgactttggacaggtgagccacggcagcctgagctgctcagttaggggaatttgggcctccagagaaagagatctgaagactgctggtgcttcctggtttcataagctcagtaagaagtctgaattcgttggaagctgatgagaatatccaggaagtcaacagacaaatgtcctcaacaattgtttctaagtaggagaacatctgtcctcggtggctttcacaggaaccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcctctaaggtaaatataaaatttttaagtgtataatgtgttaaactactgattctaattgtttctctcttttagattccaacctttggaactgaccgccaccatgtccaccgctgtgctggagaaccctgggctggggaggaaactgtcagacttcgggcaggagacttcatacattgaggataactgtaaccagaatggcgccatctctctgatcttcagcctgaaggaggaagtgggcgccctggcaaaggtgctgcgcctgtttgaggagaacgacgtgaatctgacccacatcgagtcccggccttctagactgaagaaggacgagtacgagttctttacccacctggataagcggtccctgccagccctgacaaacatcatcaagatcctgaggcacgacatcggagcaaccgtgcacgagctgtctcgggacaagaagaaggataccgtgccctggttccctcggacaatccaggagctggatagatttgccaaccagatcctgtcttacggagcagagctggacgcagatcaccctggcttcaaggacccagtgtatcgggcccggagaaagcagtttgccgatatcgcctacaattataggcacggacagccaatccctcgcgtggagtatatggaggaggagaagaagacctggggcacagtgttcaagaccctgaagagcctgtacaagacacacgcctgctacgagtataaccacatcttccccctgctggagaagtattgtggctttcacgaggacaatatccctcagctggaggacgtgagccagttcctgcagacctgcacaggctttaggctgaggccagtggcaggactgctgagctcccgggacttcctgggaggactggccttcagagtgtttcactgcacccagtacatcaggcacggctccaagccaatgtatacaccagagcccgacatctgtcacgagctgctgggccacgtgcccctgtttagcgatagatccttcgcccagttttcccaggagatcggactggcatctctgggagcacctgacgagtacatcgagaagctggccaccatctattggttcacagtggagtttggcctgtgcaagcagggcgatagcatcaaggcctacggagcaggactgctgtctagcttcggcgagctgcagtattgtctgtccgagaagccaaagctgctgcccctggagctggagaagaccgccatccagaactacaccgtgacagagttccagcccctgtactatgtggccgagtcttttaacgatgccaaggagaaggtgagaaatttcgccgccacaatccctaggcccttcagtgtgcgttacgacccttatacccagaggatcgaggtgctggataatacacagcagctgaagatcctggctgactcaatcaatagcgaaatcggaatcctgtgctccgccctgcagaaaatcaaatgagaattcaaggcctctcgagcctctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaagctttacgtacgatcgtcgactgggatgggatgtggaatccttctagatttcttttgtaatatttataaagtgctctcagcaaggtatcaaaatggcaaaattgtgagtaactatcctcctttcattttgggaagaagatgaggcatgaagagaattcagacagaaacttactcagaccaggggaggcagaaactaagcagagaggaaaatgaccaagagttagccctgggcatggaatgtgaaagaaccctaaacgtgacttggaaataatgcccaaggtatattccattctccgggatttgttggcattttcttgaggtgaagaattgcagaatacattctttaatgtgacctacatatttacccatgggaggaagtctgctcctggactcttgagattcagtcataaagcccaggccagggaaataatgtaagtctgcaggcccctgtcatcagtaggattagggagaagagttctcagtagaaaacagggaggctggagagaaaagaatggttaatgttaacgttaatataactagaaagactgcagaacttaggactgatttttatttgaatccttaaaaaaaaaatttcttatgaaaatagtacatggctcttaggagacagaacttattgtacagaggaacagcgtgagagtcagagtgatcccagaacaggtcctggctccatcctgcacatagttttggtgctgctggcaatacggtccccacaactgtgggaaggggttaggggcagggatctcatcaggaaagcataggggtttaaagttctttatagagcacttagaagattgagaatccacaaattatattaataacaaacaaagtagtgtcgtgttatatagtaaatgtgaatttgcagacacatttagggaaaagttataattaaaaaaataggctgtatatatacctgcagg

SEQ ID NO: 87 - DNA, 2141 bp, Artificial Sequence
  Synthetic polynucleotide
gcggccgcaactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtgccaccatgcccccacccaggaccggaagaggcctgctgtggctgggcctggtgctctcttccgtgtgcgtggccctgggaagcgaaacccaggccaacagcacaaccgacgccctgaatgtgctgctgatcattgtggacgatctgagaccctccctgggctgttacggcgacaaactggtgcggtccccaaacatcgaccagctggcctcccactccctgctgttccagaacgccttcgcccagcaggccgtgtgtgcccccagcagggtgagcttcctgaccggcagaagacctgacaccaccaggctgtacgactttaacagctactggcgggtgcacgccggcaatttcagcaccattcctcagtacttcaaggagaatggctacgtgacaatgtccgtgggcaaggtgtttcatcccggcattagctccaaccacaccgacgatagcccatactcctggtccttccccccctaccatccctccagcgagaagtacgagaacaccaaaacctgcagaggccctgacggagagctgcacgccaacctgctgtgccctgtggatgtcctggatgtgcccgaaggtaagggtttaagggatggttggttggtggggtattaatgtttaattacctggagcacctgcctgaaatcactttttttcaggcaccctgccagacaagcagtccacagagcaggccatccagctgctggagaagatgaagacaagcgccagccccttctttctggccgtgggataccacaagcctcacattccattccggtacccaaaagagttccagaagctgtaccctctggaaaacatcaccctggcccctgaccccgaggtgccagacgggctgcctcctgtggcctacaatccctggatggacatcagacagcgggaggatgtgcaggccctgaatatttccgtgccctatgggcccatccctgtggactttcagcggaaaatcagacagtcttactttgccagcgtgtcctacctggacacccaggtgggccgcctgctctcagccctggacgacctgcagctggccaattccaccatcatcgccttcaccagcgatcacggctgggccctgggcgagcacggggagtgggccaaatacagcaacttcgatgtggccacccacgtgcctctgattttttatgtgcccggccggacagccagcctgcccgaggccggggagaagctctttccttacctggaccctttcgactctgccagccagctgatggaacctggcagacagagcatggacctggtggagctggtgagcctcttccccactctggccggcctggctggcctgcaggtgccaccaagatgcccagtgccttctttccacgtggagctgtgtagagagggaaagaacctgctgaagcacttcagatttagagatctggaggaggatccctacctgccaggcaacccaagggagctgatcgcctacagccagtatcccagaccctctgatatcccccagtggaacagcgataagccctccctgaaagacatcaagattatgggctactccatcaggaccattgactaccggtacacagtgtgggtgggcttcaaccccgatgagtttctggccaacttcagcgacatccacgccggcgagctgtattttgtggactccgaccccctgcaggaccacaacatgtacaacgactcccagggcggcgacctgttccagctgctgatgccctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaacctgcaggtctagatacgta

SEQ ID NO: 88 - DNA, 2042 bp, Artificial Sequence
  Synthetic polynucleotide
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcctctaaggtaaatataaaatttttaagtgtataatgtgttaaactactgattctaattgtttctctcttttagattccaacctttggaactgaccgccaccatgtccaccgctgtgctggagaaccctgggctggggaggaaactgtcagacttcgggcaggagacttcatacattgaggataactgtaaccagaatggcgccatctctctgatcttcagcctgaaggaggaagtgggcgccctggcaaaggtgctgcgcctgtttgaggagaacgacgtgaatctgacccacatcgagtcccggccttctagactgaagaaggacgagtacgagttctttacccacctggataagcggtccctgccagccctgacaaacatcatcaagatcctgaggcacgacatcggagcaaccgtgcacgagctgtctcgggacaagaagaaggataccgtgccctggttccctcggacaatccaggagctggatagatttgccaaccagatcctgtcttacggagcagagctggacgcagatcaccctggcttcaaggacccagtgtatcgggcccggagaaagcagtttgccgatatcgcctacaattataggcacggacagccaatccctcgcgtggagtatatggaggaggagaagaagacctggggcacagtgttcaagaccctgaagagcctgtacaagacacacgcctgctacgagtataaccacatcttccccctgctggagaagtattgtggctttcacgaggacaatatccctcagctggaggacgtgagccagttcctgcagacctgcacaggctttaggctgaggccagtggcaggactgctgagctcccgggacttcctgggaggactggccttcagagtgtttcactgcacccagtacatcaggcacggctccaagccaatgtatacaccagagcccgacatctgtcacgagctgctgggccacgtgcccctgtttagcgatagatccttcgcccagttttcccaggagatcggactggcatctctgggagcacctgacgagtacatcgagaagctggccaccatctattggttcacagtggagtttggcctgtgcaagcagggcgatagcatcaaggcctacggagcaggactgctgtctagcttcggcgagctgcagtattgtctgtccgagaagccaaagctgctgcccctggagctggagaagaccgccatccagaactacaccgtgacagagttccagcccctgtactatgtggccgagtcttttaacgatgccaaggagaaggtgagaaatttcgccgccacaatccctaggcccttcagcgtgcggtacgacccttatacccagaggatcgaggtgctggataatacacagcagctgaagatcctggctgactcaatcaatagcgaaatcggaatcctgtgctccgccctgcagaaaatcaaatgaatgctttatttgtgaaatttgtgatgctattgctttatttgtaaccattataagctgcaataaacaagttaacaacaacaattgcattcattttatgtttcaggttcagggggaggtgtgggaggttttttaaa

SEQ ID NO: 89 - DNA, 720 bp, Artificial Sequence
  Synthetic polynucleotide
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 90 - DNA, 2162 bp, Artificial Sequence
  Synthetic polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcatatgcggccgcacgcgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccatcaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaaggcctgcaggtctagatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 91 - DNA, 2208 bp, Artificial Sequence
  Adeno-associated virus 2
ttacagattacgagtcaggtatctggtgccaatggggcgaggctctgaatacacgccattagtgtccacagtaaagtccacattaacagacttgttgtagttggaagtgtactgaatttcgggattccagcgtttgctgttttccttctgcagctcccactcgatctccacgctgacctgtcccgtggagtactgtgtgatgaaggaagcaaactttgccgcactgaaggtggtcgaaggattcgcaggtaccggggtgttcttgatgagaatctgtggaggagggtgtttaagtccgaatccacccatgaggggagaggggtgaaaatgtccgtccgtgtgtggaatctttgcccagatgggcccctgaaggtacacatctctgtcctgccagaccatgcctggaagaacgccttgtgtgttgacatctgcggtagctgcttgtctgttgcctctctggaggttggtagatacagaaccatactgctccgtagccacgggattggttgtcctgatttcctcttcgtctgtaatcatgaccttttcaatgtccacatttgttttctctgagccttgcttcccaaagatgagaaccccgctctgaggaaaaaacttttcttcatcgtccttgtggcttgccatggccgggcccggattcaccagagagtctctgccattgaggtggtacttggtagctccagtccacgagtattcactgttgttgttatccgcagatgtctttgatactcgctgctggcggtaacagggtccaggaagccagttcctagactggtcccgaatgtcactcgctccggcctgagaaaactgaagccttgactgcgtggtggttccacttggagtgtttgttctgctcaagtaatacaggtactggtcgatgagaggattcatgagacggtccagactctggctgtgagcgtagctgctgtggaaaggaacgtcctcaaaagtgtagctgaaggtaaagttgtttccggtacgcagcatctgagaaggaaagtactccaggcagtaaaatgaagagcgtcctactgcctgactcccgttgttcagggtgaggtatccatactgtggcaccatgaagacgtctgctgggaacggcgggaggcatccttgatgcgccgagccgaggacgtacgggagctggtactccgagtcagtaaacacctgaaccgtgctggtaaggttattggcaatcgtcgtcgtaccgtcattctgcgtgacctctttgacttgaatgttaaagagcttgaagttgagtctcttgggtcggaatccccagttgttgttgatgagtctttgccagtcacgtggtgaaaagtggcagtggaatctgttgaagtcaaaatacccccaaggggtgctgtagccaaagtagtgattgtcgttcgaggctcctgattggctggaaatttgtttgtagaggtggttgttgtaggtgggcagggcccaggttcgggtgctggtggtgatgactctgtcgcccatccatgtggaatcgcaatgccaatttcccgaggaattacccactccgtcggcgccctcgttattgtctgccattggtgcgccactgcctgtagccatcgtattagttcccagaccagagggggctgctggtggctgtccgagaggctgggggtcaggtactgagtctgcgtctccagtctgaccaaaattcaatctttttcttgcaggctgctggcccgcctttccggttcccgaggaggagtctggctccacaggagagtgctctaccggcctcttttttcccggagccgtcttaacaggttcctcaaccaggcccagaggttcaagaaccctctttttcgcctggaagactgctcgtccgaggttgcccccaaaagacgtatcttctttaaggcgctcctgaaactccgcgtcggcgtggttgtacttgaggtacgggttgtctccgctgtcgagctgccggtcgtaggctttgtcgtgctcgagggccgcggcgtctgcctcgttgaccggctctcccttgtcgagtccgttgaagggtccgaggtacttgtacccaggaagcacaagacccctgctgtcgtccttatgccgctctgcgggctttggtggtggtgggccaggtttgagcttccaccactgtcttattccttcagagagagtgtcctcgagccaatctggaagataaccatcggcagccat

SEQ ID NO: 92 - DNA, 4219 bp, Artificial Sequence
  Synthetic polynucleotide
ttacagattacgagtcaggtatctggtgccaatggggcgaggctctgaatacacgccattagtgtccacagtaaagtccacattaacagacttgttgtagttggaagtgtactgaatttcgggattccagcgtttgctgttttccttctgcagctcccactcgatctccacgctgacctgtcccgtggagtactgtgtgatgaaggaagcaaactttgccgcactgaaggtggtcgaaggattcgcaggtaccggggtgttcttgatgagaatctgtggaggagggtgtttaagtccgaatccacccatgaggggagaggggtgaaaatgtccgtccgtgtgtggaatctttgcccagatgggcccctgaaggtacacatctctgtcctgccagaccatgcctggaagaacgccttgtgtgttgacatctgcggtagctgcttgtctgttgcctctctggaggttggtagatacagaaccatactgctccgtagccacgggattggttgtcctgatttcctcttcgtctgtaatcatgaccttttcaatgtccacatttgttttctctgagccttgcttcccaaagatgagaaccccgctctgaggaaaaaacttttcttcatcgtccttgtggcttgccatggccgggcccggattcaccagagagtctctgccattgaggtggtacttggtagctccagtccacgagtattcactgttgttgttatccgcagatgtctttgatactcgctgctggcggtaacagggtccaggaagccagttcctagactggtcccgaatgtcactcgctccggcctgagaaaactgaagccttgactgcgtggtggttccacttggagtgtttgttctgctcaagtaatacaggtactggtcgatgagaggattcatgagacggtccagactctggctgtgagcgtagctgctgtggaaaggaacgtcctcaaaagtgtagctgaaggtaaagttgtttccggtacgcagcatctgagaaggaaagtactccaggcagtaaaatgaagagcgtcctactgcctgactcccgttgttcagggtgaggtatccatactgtggcaccatgaagacgtctgctgggaacggcgggaggcatccttgatgcgccgagccgaggacgtacgggagctggtactccgagtcagtaaacacctgaaccgtgctggtaaggttattggcaatcgtcgtcgtaccgtcattctgcgtgacctctttgacttgaatgttaaagagcttgaagttgagtctcttgggtcggaatccccagttgttgttgatgagtctttgccagtcacgtggtgaaaagtggcagtggaatctgttgaagtcaaaatacccccaaggggtgctgtagccaaagtagtgattgtcgttcgaggctcctgattggctggaaatttgtttgtagaggtggttgttgtaggtgggcagggcccaggttcgggtgctggtggtgatgactctgtcgcccatccatgtggaatcgcaatgccaatttcccgaggaattacccactccgtcggcgccctcgttattgtctgccattggtgcgccactgcctgtagccatcgtattagttcccagaccagagggggctgctggtggctgtccgagaggctgggggtcaggtactgagtctgcgtctccagtctgaccaaaattcaatctttttcttgcaggctgctggcccgcctttccggttcccgaggaggagtctggctccacaggagagtgctctaccggcctcttttttcccggagccgtcttaacaggttcctcaaccaggcccagaggttcaagaaccctctttttcgcctggaagactgctcgtccgaggttgcccccaaaagacgtatcttctttaaggcgctcctgaaactccgcgtcggcgtggttgtacttgaggtacgggttgtctccgctgtcgagctgccggtcgtaggctttgtcgtgctcgagggccgcggcgtctgcctcgttgaccggctctcccttgtcgagtccgttgaagggtccgaggtacttgtacccaggaagcacaagacccctgctgtcgtccttatgccgctctgcgggctttggtggtggtgggccaggtttgagcttccaccactgtcttattccttcagagagagtgtcctcgagccaatctggaagataaccatcggcagccatacctggtttaagtcatttattgttcaaagatgcagtcatccaaatccacattgaccagatcgcaggcagtgcaagcgtctggcacctttcccatgatatgatgaatgtagcacagtttctgatacgcctttttgacgacagaaacgggttgagattctgacacgggaaagcactctaaacagtctttctgtccgtgagtgaagcagatatttgaattctgattcattctctcgcattgtctgcagggaaacagcatcagattcatgcccacgtgacgagaacatttgttttggtacctgtctgcgtagttgatcgaagcttccgcgtctgacgtcgatggctgcgcaactgactcgcgcacccgtttgggctcacttatatctgcgtcactgggggcgggtcttttcttggctccaccctttttgacgtagaattcatgctccacctcaaccacgtgatcctttgcccaccggaaaaagtctttgacttcctgcttggtgaccttcccaaagtcatgatccagacggcgggtgagttcaaatttgaacatccggtcttgcaacggctgctggtgttcgaaggtcgttgagttcccgtcaatcacggcgcacatgttggtgttggaggtgacgatcacgggagtcgggtctatctgggccgaggacttgcatttctggtccacgcgcaccttgcttcctccgagaatggctttggccgactccacgaccttggcggtcatcttcccctcctcccaccagatcaccatcttgtcgacacagtcgttgaagggaaagttctcattggtccagtttacgcacccgtagaagggcacagtgtgggctatggcctccgcgatgttggtcttcccggtagttgcaggcccaaacagccagatggtgttcctcttgccgaactttttcgtggcccatcccagaaagacggaagccgcatattggggatcgtacccgtttagttccaaaattttataaatccgattgctggaaatgtcctccacgggctgctggcccaccaggtagtcgggggcggttttagtcaggctcataatctttcccgcattgtccaaggcagccttgatttgggaccgcgagttggaggccgcattgaaggagatgtatgaggcctggtcctcctggatccactgcttctccgaggtaatccccttgtccacgagccacccgaccagctccatgtacctggctgaagtttttgatctgatcaccggcgcatcagaattgggattctgattctctttgttctgctcctgcgtctgcgacacgtgcgtcagatgctgcgccaccaaccgtttacgctccgtgagattcaaacaggcgcttaaatactgttccatattagtccacgcccactggagctcaggctgggttttggggagcaagtaattggggatgtagcactcatccaccaccttgttcccgcctccggcgccatttctggtctttgtgaccgcgaaccagtttggcaaagtcggctcgatcccgcggtaaattctctgaatcagtttttcgcgaatctgactcaggaaacgtcccaaaaccatggatttcaccccggtggtttccacgagcacgtgcatgtggaagtagctctctcccttctcaaattgcacaaagaaaagggcctccggggccttactcacacggcgccattccgtcagaaagtcgcgctgcagcttctcggccacggtcaggggtgcctgctcaatcagattcagatccatgtcagaatctggcggcaactcccattccttctcggccacccagttcacaaagctgtcagaaatgccgggcagatgctcgtcaaggtcgctggggaccttaatcacaatctcgtaaaaccccggcatggcggctgcgcgttcaaacctcccgcttcaaaatggagaccctgcgtgctcactcgggcttaaatacccagcgtgaccacatggtgtcgcaaaatgtcgcaaaacactcacgtgacctctaatacagga

SEQ ID NO: 93 - DNA, 2211 bp, Artificial Sequence
  Synthetic polynucleotide
atggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgatccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaa

SEQ ID NO: 94 - DNA, 4263 bp, Artificial Sequence
  Synthetic polynucleotide
ggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgacttaaaccaggtatggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgatccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaa

SEQ ID NO: 95 - DNA, 2175 bp, Artificial Sequence
  Synthetic polynucleotide
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 96 - DNA, 4057 bp, Artificial Sequence
  Synthetic polynucleotide
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgacttaaaccaggtatgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 97 - DNA, 2220 bp, Artificial Sequence
  Synthetic polynucleotide
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaacctcttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtatgcttgttaa

SEQ ID NO: 98 - DNA, 4102 bp, Artificial Sequence
  Synthetic polynucleotide
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgacttaaaccaggtatggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaacctcttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtatgcttgttaa

SEQ ID NO: 99 - DNA, 2217 bp, Artificial Sequence
  Synthetic polynucleotide
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 100 - DNA, 4269 bp, Artificial Sequence
  Synthetic polynucleotide
ggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgacttaaaccaggtatggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 101 - DNA, 2211 bp, Artificial Sequence
  Synthetic polynucleotide
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 102 - DNA, 4093 bp, Artificial Sequence
  Synthetic polynucleotide
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgacttaaaccaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 103 - DNA, 2217 bp, Artificial Sequence
  Synthetic polynucleotide
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccagcagcccgcgaaaaagagactcaactttgggcagactggcgactcagagtcagtgcccgaccctcaaccaatcggagaaccccccgcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctccccacctacaacaaccacctctacaagcaaatctccaacgggacttcgggaggaagcaccaacgacaacacctacttcggctacagcaccccctgggggtattttgactttaacagattccactgccacttctcaccacgtgactggcagcgactcatcaacaacaactggggattccggcccaagagactcaacttcaagctcttcaacatccaggtcaaggaggtcacgcagaatgaaggcaccaagaccatcgccaataaccttaccagcacgattcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcaaatgctgagaacgggcaacaactttgagttcagctaccagtttgaggacgtgccttttcacagcagctacgcgcacagccaaagcctggaccggctgatgaaccccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcagcaacgcgtctccacgacactgtcgcaaaataacaacagcaactttgcctggaccggtgccaccaagtatcatctgaatggcagagactctctggtaaatcccggtgtcgctatggcaacccacaaggacgacgaagagcgattttttccgtccagcggagtcttaatgtttgggaaacagggagctggaaaagacaacgtggactatagcagcgttatgctaaccagtgaggaagaaattaaaaccaccaacccagtggccacagaacagtacggcgtggtggccgataacctgcaacagcaaaacgccgctcctattgtaggggccgtcaacagtcaaggagccttacctggcatggtctggcagaaccgggacgtgtacctgcagggtcctatctgggccaagattcctcacacggacggaaactttcatccctcgccgctgatgggaggctttggactgaaacacccgcctcctcagatcctgattaagaatacacctgttcccgcggatcctccaactaccttcagtcaagctaagctggcgtcgttcatcacgcagtacagcaccggacaggtcagcgtggaaattgaatgggagctgcagaaagaaaacagcaaacgctggaacccagagattcaatacacttccaactactacaaatctacaaatgtggactttgctgttaacacagatggcacttattctgagcctcgccccatcggcacccgttacctcacccgtaatctgtaa

SEQ ID NO: 104 - DNA, 4269 bp, Artificial Sequence
  Synthetic polynucleotide
ggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgacttaaaccaggtatggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccagcagcccgcgaaaaagagactcaactttgggcagactggcgactcagagtcagtgcccgaccctcaaccaatcggagaaccccccgcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctccccacctacaacaaccacctctacaagcaaatctccaacgggacttcgggaggaagcaccaacgacaacacctacttcggctacagcaccccctgggggtattttgactttaacagattccactgccacttctcaccacgtgactggcagcgactcatcaacaacaactggggattccggcccaagagactcaacttcaagctcttcaacatccaggtcaaggaggtcacgcagaatgaaggcaccaagaccatcgccaataaccttaccagcacgattcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcaaatgctgagaacgggcaacaactttgagttcagctaccagtttgaggacgtgccttttcacagcagctacgcgcacagccaaagcctggaccggctgatgaaccccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcagcaacgcgtctccacgacactgtcgcaaaataacaacagcaactttgcctggaccggtgccaccaagtatcatctgaatggcagagactctctggtaaatcccggtgtcgctatggcaacccacaaggacgacgaagagcgattttttccgtccagcggagtcttaatgtttgggaaacagggagctggaaaagacaacgtggactatagcagcgttatgctaaccagtgaggaagaaattaaaaccaccaacccagtggccacagaacagtacggcgtggtggccgataacctgcaacagcaaaacgccgctcctattgtaggggccgtcaacagtcaaggagccttacctggcatggtctggcagaaccgggacgtgtacctgcagggtcctatctgggccaagattcctcacacggacggaaactttcatccctcgccgctgatgggaggctttggactgaaacacccgcctcctcagatcctgattaagaatacacctgttcccgcggatcctccaactaccttcagtcaagctaagctggcgtcgttcatcacgcagtacagcaccggacaggtcagcgtggaaattgaatgggagctgcagaaagaaaacagcaaacgctggaacccagagattcaatacacttccaactactacaaatctacaaatgtggactttgctgttaacacagatggcacttattctgagcctcgccccatcggcacccgttacctcacccgtaatctgtaa

SEQ ID NO: 105 - DNA, 2217 bp, Artificial Sequence
  Synthetic polynucleotide
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctccaagcgggtgacaatccgtacctgcggtataatcacgccgacgccgagtttcaggagcgtctgcaagaagatacgtcttttgggggcaacctcgggcgcgcagtcttccaggccaaaaagcgggttctcgaacctctgggcctggttgaatcgccggttaagacggctcctggaaagaagagaccggtagagccatcaccccagcgctctccagactcctctacgggcatcggcaagaaaggccagcagcccgcaaaaaagagactcaattttgggcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgcacctgggccctgcccacctacaacaaccacctctacaagcaaatctccaacgggacctcgggaggaagcaccaacgacaacacctacttcggctacagcaccccctgggggtattttgacttcaacagattccactgccacttttcaccacgtgactggcagcgactcatcaacaacaactggggattccggcccaagaggctcaacttcaagctcttcaacatccaagtcaaggaggtcacgcagaatgaaggcaccaagaccatcgccaataaccttaccagcacgattcaggtctttacggactcggaataccagctcccgtacgtgctcggctcggcgcaccagggctgcctgcctccgttcccggcggacgtcttcatgattcctcagtacgggtacctgactctgaacaatggcagtcaggctgtgggccggtcgtccttctactgcctggagtactttccttctcaaatgctgagaacgggcaacaactttgaattcagctacaacttcgaggacgtgcccttccacagcagctacgcgcacagccagagcctggaccggctgatgaaccctctcatcgaccagtacttgtactacctgtcccggactcaaagcacgggcggtactgcaggaactcagcagttgctattttctcaggccgggcctaacaacatgtcggctcaggccaagaactggctacccggtccctgctaccggcagcaacgcgtctccacgacactgtcgcagaacaacaacagcaactttgcctggacgggtgccaccaagtatcatctgaatggcagagactctctggtgaatcctggcgttgccatggctacccacaaggacgacgaagagcgattttttccatccagcggagtcttaatgtttgggaaacagggagctggaaaagacaacgtggactatagcagcgtgatgctaaccagcgaggaagaaataaagaccaccaacccagtggccacagaacagtacggcgtggtggccgataacctgcaacagcaaaacgccgctcctattgtaggggccgtcaatagtcaaggagccttacctgggatggtgtggcagaaccgggacgtgtacctgcagggtcccatctgggccaagattcctcatacggacggcaactttcatccctcgccgctgatgggaggctttggactgaagcatccgcctcctcagatcctgattaaaaacacacctgttcccgcggatcctccgaccaccttcaatcaggccaagctggcttctttcatcacgcagtacagtaccggccaggtcagcgtggagatcgagtgggagctgcagaaggagaacagcaaacgctggaacccagagattcagtacacttccaactactacaaatctacaaatgtggactttgctgtcaatactgagggtacttattccgagcctcgccccattggcacccgttacctcacccgtaatctgtaa

SEQ ID NO: 106 - DNA, 4099 bp, Artificial Sequence
  Synthetic polynucleotide
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgacttaaaccaggtatggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctccaagcgggtgacaatccgtacctgcggtataatcacgccgacgccgagtttcaggagcgtctgcaagaagatacgtcttttgggggcaacctcgggcgcgcagtcttccaggccaaaaagcgggttctcgaacctctgggcctggttgaatcgccggttaagacggctcctggaaagaagagaccggtagagccatcaccccagcgctctccagactcctctacgggcatcggcaagaaaggccagcagcccgcaaaaaagagactcaattttgggcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgcacctgggccctgcccacctacaacaaccacctctacaagcaaatctccaacgggacctcgggaggaagcaccaacgacaacacctacttcggctacagcaccccctgggggtattttgacttcaacagattccactgccacttttcaccacgtgactggcagcgactcatcaacaacaactggggattccggcccaagaggctcaacttcaagctcttcaacatccaagtcaaggaggtcacgcagaatgaaggcaccaagaccatcgccaataaccttaccagcacgattcaggtctttacggactcggaataccagctcccgtacgtgctcggctcggcgcaccagggctgcctgcctccgttcccggcggacgtcttcatgattcctcagtacgggtacctgactctgaacaatggcagtcaggctgtgggccggtcgtccttctactgcctggagtactttccttctcaaatgctgagaacgggcaacaactttgaattcagctacaacttcgaggacgtgcccttccacagcagctacgcgcacagccagagcctggaccggctgatgaaccctctcatcgaccagtacttgtactacctgtcccggactcaaagcacgggcggtactgcaggaactcagcagttgctattttctcaggccgggcctaacaacatgtcggctcaggccaagaactggctacccggtccctgctaccggcagcaacgcgtctccacgacactgtcgcagaacaacaacagcaactttgcctggacgggtgccaccaagtatcatctgaatggcagagactctctggtgaatcctggcgttgccatggctacccacaaggacgacgaagagcgattttttccatccagcggagtcttaatgtttgggaaacagggagctggaaaagacaacgtggactatagcagcgtgatgctaaccagcgaggaagaaataaagaccaccaacccagtggccacagaacagtacggcgtggtggccgataacctgcaacagcaaaacgccgctcctattgtaggggccgtcaatagtcaaggagccttacctgggatggtgtggcagaaccgggacgtgtacctgcagggtcccatctgggccaagattcctcatacggacggcaactttcatccctcgccgctgatgggaggctttggactgaagcatccgcctcctcagatcctgattaaaaacacacctgttcccgcggatcctccgaccaccttcaatcaggccaagctggcttctttcatcacgcagtacagtaccggccaggtcagcgtggagatcgagtgggagctgcagaaggagaacagcaaacgctggaacccagagattcagtacacttccaactactacaaatctacaaatgtggactttgctgtcaatactgagggtacttattccgagcctcgccccattggcacccgttacctcacccgtaatctgtaa